(19) United States
(12) Patent Application Publication
(10) Pub. No.: US 2018/0148789 A1
(43) Pub. Date: May 31, 2018

(54) METHODS FOR TREATING AND ASSESSING TUMOR INVASION AND METASTASIS

(71) Applicant: UNIVERSITY OF SOUTHERN CALIFORNIA, Los Angeles, CA (US)

(72) Inventors: Andrew C. WEITZ, Pasadena, CA (US); Nan Sook LEE, Pasadena, CA (US); Robert H. CHOW, Pasadena, CA (US)

(21) Appl. No.: 15/532,303

(22) PCT Filed: Dec. 1, 2015
(86) PCT No.: PCT/US2015/063299

§ 371 Date: Jun. 1, 2017

Related U.S. Application Data
(60) Provisional application No. 62/086,186, filed on Dec. 1, 2014.

(51) Int. Cl. C12Q 1/6886; C12N 15/113; A61P 35/00

(52) U.S. Cl. CPC C12Q 1/6886; C12N 15/113; C12N 2310/14; C12Q 2600/158; C12Q 2600/118; A61P 35/00

(57) ABSTRACT

Provided herein are methods, such as diagnostic methods, for evaluating a tissue sample, such as a biopsy or cytology specimen of a subject to determine a likelihood of metastasis, a risk of tumor or cancer occurrence, or combinations thereof and provided herein are methods, such as a screening method, for determining the efficacy of a drug in reducing cell invasiveness. Also provided are methods of treatment, drugs, primers, and kits.

### CROSS REFERENCE

This application claims priority to U.S. provisional application 62/086,186, filed on Dec. 1, 2014, which is entirely incorporated herein by reference.

### STATEMENT REGARDING FEDERALLY SPONSORED RESEARCH

This invention was made with government support under National Institutes of Health grant P41-EB2182. The government has certain rights in the invention.

### BACKGROUND

Cancer is the second leading cause of death in the U.S., accounting for nearly 1 in every 4 deaths. Once a patient is diagnosed with cancer, doctors must establish how aggressively to treat the patient. Choosing the appropriate treatment requires determining whether the cancer is invasive (i.e., able to spread into surrounding tissue). Invasion is a prerequisite for metastasis, the process by which cells break away from the primary tumor and form a secondary tumor in different organ. Metastasis is the cause for 9 out of 10 cancer deaths. Tumor invasion is crucial to determine, as it signals a worse prognosis and may lead to widespread dissemination by metastasis. Early identification invasiveness is crucial to establish the aggressiveness of cancer therapy. RE1-Silencing Transcription factor (REST) has a well-established role in regulating transcription of genes important for neuronal development¹. Its role in cancer, though significant, is less well understood².

### SUMMARY OF THE INVENTION

In some embodiments, provided herein are various methods for determining the presence of invasive cells in a cell sample or tissue and methods for selectively inducing apoptosis or cell death in at least a subset of cells of a tissue in a subject.

An aspect of the present disclosure provides a method, the method comprising (i) assaying a subject's tissue sample for an expression level of one or more oligonucleotide sequences, wherein each of the one or more oligonucleotide sequences independently can comprise: (a) at least 70% homology to or at least 70% of the nucleobases or any combination

thereof of at least a portion of a (RE-1)-Silencing transcription factor 003 (REST-003) sequence or fragment thereof, (b) at least 70% homology to or at least 70% of the nucleobases or any combination thereof of at least a portion of one or more REST-003-mediated oligonucleotide

sequences or fragments thereof, or (c) any combination thereof, and (ii) comparing the expression level obtained in (i) to an expression level of the one or more oligonucleotide sequences of a control.

In some embodiments, the method can further comprise determining a likelihood of metastasis, a risk of tumor or cancer occurrence or reccurrence, an invasion potential, an effectiveness of a cancer or tumor treatment, an effectiveness of a drug, a longitudinal course of a cancer or tumor treatment regime, or any combination thereof, in the subject based on the comparing.

In some embodiments, the method can be for evaluating the tissue sample of the subject to determine the likelihood of metastasis, the risk of tumor or cancer occurrence or reccurrence, the invasion potential, the effectiveness of a cancer or tumor treatment, the effectiveness of a drug, the longitudinal course of a cancer or tumor treatment regime, or any combination thereof in the subject.

In some embodiments, each of the one or more oligonucleotide sequences can independently comprise (i) at least 80% homology to or at least 80% of the nucleobases or any combination thereof of at least a portion of the REST-003 sequence or fragment thereof, (ii) at least 80% homology to or at least 80% of the nucleobases or any combination thereof of at least a portion of the one or more REST-003-mediated oligonucleotide sequences or fragments thereof, or (iii) any combination thereof.

In some embodiments, each of the one or more oligonucleotide sequences can independently comprise (i) at least 95% homology to or at least 95% of the nucleobases or any combination thereof of at least a portion of the REST-003 sequence or fragment thereof, (ii) at least 95% homology to or at least 95% of the nucleobases or any combination thereof of at least a portion of the one or more REST-003-mediated oligonucleotide sequences or fragments thereof, or (iii) any combination thereof.

In some embodiments, the control can be a non-cancerous tissue sample, a non-tumor tissue sample, or a combination thereof.

In some embodiments, the tissue sample can be an excised tissue, a biopsy, a fine needle aspirate, a cytology specimen, a tissue washing, or any combination thereof.

In some embodiments, the tissue sample can comprise a tumor cell, a cancer cell, a non-tumor cell, a non-cancerous cell, or any combination thereof.

In some embodiments, the tissue sample can be a breast tissue or a bladder tissue.

In some embodiments, the tissue sample can be a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

In some embodiments, the method further can further comprise determining a risk of cancer occurrence or reccurrence. In some embodiments, the risk of cancer occurrence is a risk of breast cancer occurrence or a bladder cancer occurrence.

In some embodiments, the method can further comprise determining a risk of tumor occurrence. In some embodiments, the risk of tumor occurrence is a breast tumor occurrence or a bladder tumor occurrence.

In some embodiments, the method can further comprise determining a risk of tumor or cancer occurrence or reccurrence. In some embodiments, the risk of tumor or cancer occurrence or reccurrence is a risk of an occurrence of an adrenal cortical cancer, anal cancer, aplastic anemia, bile duct cancer, bladder cancer, bone cancer, bone metastasis, central nervous system (CNS) cancer, peripheral nervous system (PNS) cancer, breast cancer, Castleman's disease, cervical cancer, childhood Non-Hodgkin's lymphoma, lymphoma, colon and rectum cancer, endometrial cancer, esophagus cancer, Ewing's sarcoma, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, melanoma skin cancer, non-melanoma skin cancer, stomach cancer, testicular cancer, thymus cancer, uterine cancer, vaginal cancer, vulvar cancer, Waldenstrom's macroglobulinemia, or any combination thereof.

In some embodiments, the subject can be a cancer patient or a tumor patient or a cancer and tumor patient.

In some embodiments, the comparing, the determining, or a combination thereof can be computer implemented.

In some embodiments, the computer implementation can comprise conducting at least a portion of the comparing, at least a portion of the determining, or a combination thereof with a processor or microprocessor.

In some embodiments, the expression level of the one or more oligonucleotide sequences of the control can be an average expression level.

In some embodiments, the expression level of the one or more oligonucleotide sequences of the control can be a reference value obtained from a database.

In some embodiments, the database can comprise an average expression level for at least one of the one or more oligonucleotide sequences. In some embodiments, the average expression level can be obtained from: at least 1, at least 5, at least 10, at least 15, or at least 20 non-cancerous, non-tumorous, or non-cancerous and non-tumorous tissue samples.

In some embodiments, the database can comprise an average expression level for each of the one or more oligonucleotide sequences. In some embodiments, the average expression level can be obtained from: at least 1, at least 5, at least 10, at least 15, or at least 20 noncancerous, non-tumorous, or noncancerous and non-tumorous tissue samples.

In some embodiments, prior to (i), tumor cells, cancer cells, or a combination thereof can be (a) identified in the tissue sample, (b) can be enriched in the sample, or (c) a combination thereof.

In some embodiments, the method further can comprise identifying tumor cells, cancer cells, or a combination thereof. In some embodiments, the identification can comprise staining the tissue sample for one or more cell surface markers.

In some embodiments, the method further can comprise enriching the sample for tumor cells, cancer cells, or a combination thereof. In some embodiments, the enrichment can comprise sorting the tissue sample for one or more cell surface markers.

In some embodiments, the one or more cell-surface markers can comprise CD19, CD20, CD24, CD34, CD38, CD44, CD90, CD133, epithelial cell adhesion molecule (EpCAM), ATP-binding cassette transporter B5 (ABCBS), adhesion G-protein coupled receptor (GPR116), or any combination thereof. In some embodiments, the one or more cell-surface markers can comprise CD44, GPR116, or a combination thereof.

In some embodiments, the method can be conducted prior to an operation on a tumor tissue or a cancer tissue of the subject.

In some embodiments, the method can be conducted prior to the subject receiving a positive cancer diagnosis or a positive tumor diagnosis.

In some embodiments, the method can be conducted after the subject receives a positive cancer diagnosis or a positive tumor diagnosis.

In some embodiments, the method further can comprise, prior to (i) obtaining the tissue sample from the subject.

In some embodiments, the method further can comprise performing at least one other diagnostic method.

In some embodiments, the at least one other diagnostic method can comprise performing a tissue biopsy, an endoscopy, a diagnostic imaging, a blood test, a genetic analysis, or any combination thereof.

In some embodiments, the assaying can comprise an array hybridization, a serial analysis of gene expression (SAGE), an enzyme linked immuno-absorbance assay, a mass-spectrometry, an immuno-histochemistry, a blotting, a RNA sequencing, a DNA sequencing, a next generation (Next-Gen) sequencing, a nanopore sequencing, a pyrosequencing, a nanostring sequencing, a microarray, a reverse transcriptase polymerase chain reaction (RT-PCR), a quantitative RT-PCR (qRT-PCR), a real-time reverse transcriptase PCR (RT-rtPCR), a nested PCR, a high-throughput RNA sequencing (RNA-seq), or any combination thereof using markers that can be selected for the one or more oligonucleotide sequences.

In some embodiments, the markers can be primers.

In some embodiments, each of the markers independently can comprise a sequence with at least 70% sequence homology to or at least 70% of the nucleobases or combination thereof of AGTGTCGGGGCGACTCCCG, 70% sequence homology to or at least 70% of the nucleobases or combination thereof of GGCATTCCTAACTGAAATAGG, any fragment thereof, or any combination thereof.

In some embodiments, each of the markers independently can comprise a sequence with at least 70% sequence homology to or at least 70% of the nucleobases or combination thereof of: AGTGTCGGGGCGACTCCCG, GTCGATGTTGGGCCAAATTACCCAATAGC, GTAAATGTGTGCAGTGAGCGGGC, CATTCGGCCATTTTCTCAAAATAC, ATACCAAACACAAAGCAGCTCTTTG, GGCGACTCCCGCGAGTTGGTGTG, GGCATTCCTAACTGAAATAGG, any fragment thereof, or any combination thereof.

In some embodiments, a length of each of the one or more oligonucleotide sequences can be from 70 to 150 nucleotides.

In some embodiments, a length of each of the one or more oligonucleotide sequences can be from 30 to 200 nucleotides.

In some embodiments, the one or more oligonucleotide sequences can comprise a sequence from an E1 region to an E3 region of the REST-003 sequence or fragment thereof. In some embodiments, the one or more oligonucleotide sequences can comprise a sequence from an E1-3 region to an E2 region of the REST-003 sequence or fragment thereof. In some embodiments, the one or more oligonucleotide sequences can comprise a sequence from an E1-3 region. In some embodiments, the one or more oligonucleotide sequences can comprise a sequence from an E2 region. In some embodiments, the one or more oligonucleotide sequences can comprise a sequence from an E1-3 region and an E2 region. In some embodiments, the one or more oligonucleotide sequences can comprise a sequence from an E1-3 region, from an E2 region, from an E1-3 and E2 region, or any combination thereof.

In some embodiments, the method can further comprise determining the effectiveness of a cancer or tumor treatment. In some embodiments, the effectiveness of the cancer or tumor treatment can indicate an effectiveness of a drug alone or in combination with other treatment methods.

In some embodiments, the method can further comprise assaying one or more tissue samples from a subject at one or more time points and comparing expression levels obtained at the one or more time points to the control.

In some embodiments, the one or more time points can be different.

In some embodiments, the one or more time points can comprise a time point before drug administration and one or more time points after drug administration.

In some embodiments, the drug can be an anti-cancer drug, an anti-tumor drug, or combination thereof.

In some embodiments, the drug can be a preclinical stage drug, a clinical stage drug, or a drug approved by a regulatory agency.

In some embodiments, the drug can be a chemo-therapeutic drug, a small molecule or salt thereof, a small interfering RNA (siRNA), a short hairpin RNA (shRNA), an antisense RNA (asRNA), a ribozyme, an antibody or fragment thereof, an aptamer, a polypeptide, or any combination thereof.

In some embodiments, the drug can be the siRNA drug. In some embodiments, the siRNA drug can have at least 70% sequence homology to or at least 70% of the nucleobases or combination thereof of: GCAAAGAGCUGCUUUGUGUUUGGUA, UUUGCAAAGAGCUGCUUUGUGUUUGGU, any fragment thereof, or any combination thereof.

In some embodiments, the drug can be a chemotherapeutic drug, a tyrosine kinase inhibitor, an antibody or fragment thereof, a small molecule, an alkylating agent, an antimetabolite, an antimicrobial, a plant alkaloid, a topoisomerase inhibitor, any salt thereof, or any combination thereof.

In some embodiments, the drug can be Abiraterone Acetate, Abitrexate (Methotrexate), Abraxane (Paclitaxel Albumin-stabilized Nanoparticle Formulation), Adcetris (Brentuximab Vedotin), Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, Becenum (Carmustine), Beleodaq (Belinostat), Belinostat, Bendamustine Hydrochloride, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, Campath (Alemtuzumab), Camptosar (Irinotecan Hydrochloride), Capecitabine, CAPDX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), Cobimetinib, Cometriq (Cabozantinib-S-Malate), Cosmegen (Dactinomycin), Cotellic (Cobimetinib), Crizotinib, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, 5-FU (Fluorouracil), Fareston (Toremifene), Farydak (Panobinostat), Faslodex (Fulvestrant), Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Margibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), Ofatumumab, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Revlimid (Lenalidomide), Rheumatrex (Methotrexate), Rituxan (Rituximab), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thioguanine, Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), any salt thereof, or any combination thereof.

In some embodiments, the one or more REST-003-mediated oligonucleotide sequences or fragments thereof can comprise less than about 20 different genes.

In some embodiments, each of the one or more oligonucleotide sequences independently can comprise at least 70% homology to or at least 70% of the nucleobases or any combination thereof of at least a portion of one or more REST-003-mediated oligonucleotides sequences or fragments thereof. In some embodiments, the one or more oligonucleotides can comprise a sequence encoding a gene selected from the group consisting of PLEC, MAGED1, SYK, STK35, ANXA10, EHF, SLC35B2, CUL4A, EPCAM, MTMR4, fragments thereof, and any combinations thereof.

In some embodiments, an expression level of the one or more REST-003-mediated oligonucleotide sequences or fragments thereof that can be at least about 10% higher than the expression level of the control indicates a likelihood of metastasis, a risk of tumor or cancer occurrence or reccurrence, or a combination thereof, in the subject.

In some embodiments, the at least about 10% higher expression level of the one or more REST-003-mediated oligonucleotide sequences or fragments thereof can be subtracted from the expression level of the control, a result of which can be divided by the expression level of the control and multiplied by 100.

In some embodiments, the one or more REST-003-mediated oligonucleotide sequences or fragments thereof can comprise from about 10 different genes to about 70 different genes.

In some embodiments, each of the one or more oligonucleotide sequences independently can comprise at least 70% homology to or at least 70% of the nucleobases or any combination thereof of at least a portion of one or more REST-003-mediated oligonucleotides sequences or fragments thereof. In some embodiments, the one or more oligonucleotides can comprise a sequence encoding a gene selected from the group consisting of IFNL1, CXCL10, IFNB1, CXCL11, CCR1, GBP5, APOL3, GBP4, C1S, CASP1, XAF1, CCL5, IDO1, IRG1, GBP1, TNFSF10, CD274, RTP4, IFIT2, TFPI2, APOL1, GBP1P1, BST2, IFIT3, TGFBI, TRIM22, PSAT1, RSAD2, CEACAM1, GBP2, TMEM171, IL8, TLR3, CBX1, OASL, SERPINE1, MMP13, IL1B, HERC5, FNDC3A, CMPK2, ARL6IP1, PGAM1, TAP1, PMAIP1, IL6, fragments thereof, and any combination thereof.

In some embodiments, an expression level of the one or more REST-003-mediated oligonucleotide sequences or fragments thereof that can be at least about 0.5% lower than an expression level of the control indicates a likelihood of metastasis, a risk of tumor or cancer occurrence or reccurrence, or a combination thereof, in the subject.

In some embodiments, the at least about 0.5% lower than expression level of the one or more REST-003-mediated oligonucleotide sequences or fragments thereof can be subtracted from the expression level of the control, a result of which can be divided by the expression level of the control and multiplied by 100.

Another aspect of the present disclosure provides a method, the method can comprise: (i) administering a drug to a first set of cells of a tissue sample; (ii) assaying the first set of cells and a second set of cells of the tissue sample for an expression level of one or more oligonucleotide sequences, wherein each of the one or more oligonucleotide sequences independently can comprise: (a) at least 70% homology to or at least 70% of the nucleobases or any combination thereof of at least a portion of a (RE-1)-Silencing transcription factor 003 (REST-003) sequence or fragment thereof, (b) at least 70% homology to or at least 70% of the nucleobases or any combination thereof of at least a portion of one or more REST-003-mediated oligonucleotide sequences or fragments thereof, or (c) any combination thereof, and (iii) comparing the expression level of the one or more oligonucleotide sequences of the first set of cells to the expression level of the one or more oligonucleotide sequences of the second set of cells.

In some embodiments, the method further can comprise determining an efficacy of the drug in reducing cell invasiveness, an effectiveness of the drug on treating a patient, or a combination thereof, based on the comparing.

In some embodiments, the comparing, the determining, or a combination thereof can be computer implemented.

In some embodiments, the computer implementation can comprise conducting at least a portion of the comparing, at least a portion of the determining, or a combination thereof with a processor or microprocessor.

In some embodiments, the drug can be an anti-cancer drug, an anti-tumor drug, or a combination thereof.

In some embodiments, the drug can be a preclinical stage drug, a clinical stage drug, or a drug approved by a regulatory agency.

In some embodiments, the drug can be a small molecule or salt thereof, a small interfering RNA (siRNA), a short hairpin RNA (shRNA), an antisense RNA (asRNA), a ribozyme, an antibody or fragment thereof, an aptamer, a polypeptide, a chemo-therapeutic agent, or any combination thereof.

In some embodiments, the drug can be a chemotherapeutic agent, a tyrosine kinase inhibitor, an antibody or fragment thereof, a small molecule, an alkylating agent, an antimetabolite, an antibiotic, a plant alkaloid, or a topoisomerase inhibitor, any salt thereof, or any combination thereof.

In some embodiments, the assaying can comprise an array hybridization, a serial analysis of gene expression (SAGE), an enzyme linked immuno-absorbance assay, a mass-spectrometry, an immuno-histochemistry, a blotting, a RNA sequencing, a DNA sequencing, a next generation (Next-Gen) sequencing, a nanopore sequencing, a pyrosequencing, a nanostring sequencing, a microarray, a reverse transcriptase polymerase chain reaction (RT-PCR), a quantitative RT-PCR (qRT-PCR), a real-time reverse transcriptase PCR (RT-rtPCR), a nested PCR, a high-throughput RNA sequencing (RNA-seq), or any combination thereof using markers that can be selected for the one or more oligonucleotide sequences.

In some embodiments, the markers can be primers.

In some embodiments, each of the markers independently can comprise a sequence with at least 70% sequence homology to or at least 70% of the nucleobases or any combination thereof of AGTGTCGGGGCGACTCCCG, 70% sequence homology to or at least 70% of the nucleobases or any combination thereof of GGCATTCCTAACTGAAATAGG, any fragment thereof, or any combination thereof.

In some embodiments, the one or more REST-003-mediated oligonucleotide sequences or fragments thereof can comprise less than about 20 different genes.

In some embodiments, each of the one or more oligonucleotide sequences can independently comprise at least 70% homology to or at least 70% of the nucleobases or any combination thereof of at least a portion of one or more REST-003-mediated oligonucleotides sequences or fragments thereof. In some embodiments, the one or more oligonucleotides can comprise a sequence encoding a gene selected from the group consisting of PLEC, MAGED1, SYK, STK35, ANXA10, EHF, SLC35B2, CUL4A, EPCAM, MTMR4, fragments thereof, and any combinations thereof.

In some embodiments, an expression level of the one or more REST-003-mediated oligonucleotide sequences or fragments thereof that can be at least about 10% higher than the expression level of the control can indicate a likelihood of metastasis, a risk of tumor or cancer occurrence or reccurrence, or a combination thereof, in the subject.

In some embodiments, the at least about 10% higher expression level of the one or more REST-003-mediated oligonucleotide sequences or fragments thereof can be subtracted from the expression level of the control, a result of which can be divided by the expression level of the control and multiplied by 100.

In some embodiments, the one or more REST-003-mediated oligonucleotide sequences or fragments thereof can comprise from about 10 different genes to about 70 different genes.

In some embodiments, each of the one or more oligonucleotide sequences independently can comprise at least 70% homology to or at least 70% of the nucleobases of at least a portion of one or more REST-003-mediated oligonucleotides sequences or fragments thereof. In some embodiments, the one or more oligonucleotides can comprise a sequence encoding a gene selected from the group consisting of IFNL1, CXCL10, IFNB1, CXCL11, CCR1, GBP5, APOL3, GBP4, C1S, CASP1, XAF1, CCL5, IDO1, IRG1, GBP1, TNFSF10, CD274, RTP4, IFIT2, TFPI2, APOL1, GBP1P1, BST2, IFIT3, TGFBI, TRIM22, PSAT1, RSAD2, CEACAM1, GBP2, TMEM171, IL8, TLR3, CBX1, OASL, SERPINE1, MMP13, IL1B, HERC5, FNDC3A, CMPK2, ARL6IP1, PGAM1, TAP1, PMAIP1, IL6, fragments thereof, and any combination thereof.

In some embodiments, an expression level of the one or more REST-003-mediated oligonucleotide sequences or fragments thereof that can be at least about 0.5% lower than an expression level of the control can indicate a likelihood of metastasis, a risk of tumor or cancer occurrence or reccurrence, or a combination thereof, in the subject.

In some embodiments, the at least about 0.5% lower than expression level of the one or more REST-003-mediated oligonucleotide sequences or fragments thereof can be subtracted from the expression level of the control, a result of which can be divided by the expression level of the control and multiplied by 100.

In some embodiments, the method further can comprise communicating a result from the method through a communication media.

In some embodiments, the communication media can be a phone, a cell phone, an email, a text, a facsimile, an electronic health record, a mail, a website, a social media platform, a telegraph, a telegram, or any combination thereof.

In some embodiments, the method further can comprise displaying a result from the method using a screen.

In some embodiments, the screen can be a digital screen.

In some embodiments, the subject can be a subject in need thereof.

Another aspect of the present disclosure can provide a kit comprising instructions for use and one or more markers. In some embodiments, each of the one or more markers can independently comprise at least 70% sequence homology to or at least 70% of the nucleobases or combination thereof of: AGTGTCGGGGCGACTCCCG, GTCGATGTTGGGCCAAATTACCCAATAGC, GTAAATGTGTGCAGTGAGCGGGC, CATTCGGCCATTTTCTCAAAATAC, ATACCAAACACAAAGCAGCTCTTTG, GGCGACTCCCGCGAGTTGGTGTG, GGCATTCCTAACTGAAATAGG, or any fragment thereof, or any combination thereof.

In some embodiments, a drug can be found by the methods disclosed herein.

Another aspect of the present disclosure provides a method of determining a course of treatment for a subject. In some embodiments, the method can comprise employing any one of the methods disclosed herein.

Another aspect of the present disclosure provides a method for diagnosing a subject. In some embodiments, the method can comprise employing any one of the methods disclosed herein.

In some embodiments, the method can further comprise performing a reverse transcription and amplifying the reverse transcribed products.

Another aspect of the present disclosure provides a method of determining a resistance to a drug of one or more cancer cells, tumor cells, or a combination thereof in a tissue sample. In some embodiments, the method can comprise employing any one of the methods disclosed herein.

Another aspect of the present disclosure provides a method of treating a subject. In some embodiments, the method can comprise administrating a small molecule, an antibody or fragment thereof, an siRNA, an aptamer, or any combination thereof to the subject. In some embodiments, the small molecule, the antibody or fragment thereof, the siRNA, the aptamer, or any combination thereof can bind to at least a portion of the REST-003 sequence or fragment thereof.

Another aspect of the present disclosure provides a method of treating a subject. In some embodiments, the method can comprise administrating a drug to the subject. In some embodiments, the drug comprises at least 70% sequence homology to or at least 70% of the nucleobases or combination thereof of: GCAAAGAGCUGCUUUGUGUUUGGUA, UUUGCAAAGAGCUGCUUUGUGUUUGGU, any fragment thereof, or any combination thereof.

Another aspect of the present disclosure provides a method of treating a subject. In some embodiments, the method can comprise administrating a drug to the subject. In some embodiments, the drug can comprise at least 80% sequence homology to or at least 80% of the nucleobases or combination thereof of: GCAAAGAGCUGCUUUGUGUUUGGUA, UUUGCAAAGAGCUGCUUUGUGUUUGGU, any fragment thereof, or any combination thereof.

Another aspect of the present disclosure provides a method of treating a subject. In some embodiments, the method can comprise administrating a drug to the subject. In some embodiments, the drug can comprise at least 95% sequence homology to or at least 95% of the nucleobases or combination thereof of: GCAAAGAGCUGCUUUGUGUUUGGUA, UUUGCAAAGAGCUGCUUUGUGUUUGGU, any fragment thereof, or any combination thereof.

In some embodiments, the subject can be a cancer patient, a tumor patient, or a cancer and tumor patient.

In some embodiments, the drug can be an anti-cancer drug, an anti-tumor drug, or an anti-cancer and anti-tumor drug.

In some embodiments, the method can further comprise administering at least one cancer or tumor treatment.

In some embodiments, the at least one cancer or tumor treatment can be a surgery, a nutrition regime, a physical activity, a radiation treatment, a chemotherapy, an immunotherapy, a cell transplantation, a blood fusion, or any combination thereof.

Another aspect of the present disclosure provides a pharmaceutical composition comprising one or more oligonucleotide sequences. In some embodiments, the one or more oligonucleotide sequences can comprise at least 70% sequence homology to or at least 70% of the nucleobases or combination thereof of: GCAAAGAGCUGCUUUGUGUUUGGUA, UUUGCAAAGAGCUGCUUUGUGUUUGGU, any fragment thereof, or any combination thereof.

Another aspect of the present disclosure provides a pharmaceutical composition comprising a small molecule, an antibody or fragment thereof, an siRNA, an aptamer, or any combination thereof. In some embodiments, the small molecule, the antibody or fragment thereof, the siRNA, the aptamer, or any combination thereof can bind to at least a portion of a REST-003 sequence or fragment thereof.

Another aspect of the present disclosure provides a composition comprising one or more sequences. In some embodiments, the one or more sequences can comprise at least 70% sequence homology to or at least 70% of the nucleobases or combination thereof of: GCAAAGAGCUGCUUUGUGUUUGGUA, UUUGCAAAGAGCUGCUUUGUGUUUGGU, any fragment thereof, or any combination thereof.

In some embodiments, the method can further comprise administering at least one cancer treatment or tumor treatment to the subject.

In some embodiments, the administering can occur after the determining.

In some embodiments, the method can further comprise administering at least one cancer treatment or tumor treatment to a subject.

In some embodiments, the administering can occur after the determining.

In some embodiments, the at least one cancer or tumor treatment can be a surgery, a nutrition regime, a physical activity, a radiation treatment, a chemotherapy, a immunotherapy, a cell transplantation, a blood fusion, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows altering REST-003 mncRNA expression in si-REST-treated MCF-7 and REST-overexpressed MDA-MB-231 cells.

FIG. 2 shows the effect of si-REST-003 on MDA-MB-231 invasiveness and SRRM3 expression data from RNA-seq experiments on si-REST-treated MCF-7 and REST-overexpressed MDA-MB-231 cells.

FIG. 3 shows expression pattern of REST-003 and its downregulating effect on MDA-MB-231 cells.

FIG. 4 shows differences in REST expression and invasiveness between MCF-7 and MDA-MB-231 cells.

FIG. 5 shows bioinformatics at the REST gene locus.

FIG. 6 shows EnsEMBL_Web_Component_Gene_SpliceImage-Homo_sapiens-Gene-Splice-73-ENSG00000084093.

FIG. 7 shows the effect of REST downregulation in MCF-7.

FIG. 8 shows the positive correlation between REST-003 expression and invasiveness in several breast cancer cell lines and bladder cancer cell lines.

FIG. 9 shows a northern gel picture of differential expression of REST-003 in MCF-7 and MDA-MB-231 cells.

FIG. 10 shows upregulated genes and their pathways following si-REST-003 treatment in MDA-MB-231 cells.

FIG. 11 shows transcript variants.

FIG. 12 shows the primers and si-RNAs used.

FIG. 13 shows averaged reads of downregulated gene expression.

The file of this patent contains at least one drawing executed in color. Copies of this patent with color drawings will be provided to the Office upon request with the payment of the necessary fee.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about” means the referenced numeric indication plus or minus 15% of that referenced numeric indication.

The term “cancer cell,” as used herein, generally refers to a cell, such as an abnormal cell, that divides at a rate faster than a non-cancer cell, a cell that invades a tissue space or metastasizes to another part of a body, or a combination thereof. A cancer cell may have the ability to invade a tissue space or metastasize. A cancer cell may spread to other parts of a body, such as migrating through the blood and lymph systems. A cell, such as a stem cell, may become a cancer cell.

The term “tumor cell,” as used herein, generally refers to a cell that is part of a mass, such as an abnormal mass. The mass can be a solid mass, a liquid mass, or a solid and liquid mass. A tumor cell may be part of a tumor or neoplasm. A tumor cell may be benign or malignant. A tumor may be localized to a tissue or may metastasize.

The term “homology,” as used herein, generally refers to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions are then compared, and the percent identity between the two sequences is a function of the number of identical positions shared by the sequences (i.e., % homology=# of identical positions/total # of positions×100). For example, a position in the first sequence is occupied by the same nucleotide as the corresponding position in the second sequence, then the molecules are identical at that position. The percent homology between the two sequences is a function of the number of identical positions shared by the sequences, taking into account the number of gaps, and the length of each gap, which need to be introduced for optimal alignment of the two sequences. In some embodiments, the length of a sequence aligned for comparison purposes is at least 30%, at least 40%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90-5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “fragment,” as used herein, generally refers to a portion of a sequence, a subset that is shorter than a full length sequence. A fragment may be a portion of a gene. A fragment may be a portion of a peptide or protein. A fragment may be a portion of an amino acid sequence. A fragment may be a portion of an oligonucleotide sequence. A fragment may be less than 20, 30, 40, 50 amino acids in length. A fragment may be less than 20, 30, 40, 50 oligonucleotides in length.

The term “REST-003-mediated oligonucleotides sequence,” as used herein, generally refers to an oligonucleotide sequence that may be co-expressed with expression of REST-003, may be in the same signaling pathway or gene regulatory network as REST-003, may be functionally connected, or combinations thereof. RNA-sequencing data analysis, such as co-expression analysis, may generate analysis of oligonucleotides sequences of a tissue sample that may be classified as REST-003-mediated oligonucleotide sequences.

The term “average,” as used herein, generally refers to a number expressing the central or typical value in a data set. The average can be the median of the data set. The average can be the mean of the data set. The mean can be the sum of values divided by the total number of values. The median can be the central or middle value in a set of values.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about 1, 2, 5, 10, 20, 30, 40, 50, 60, 65, 70, 75, or about 80 years of age. The subject may have or be suspected of having a disease, such as a cancer or a tumor. The subject may be a patient being treated for a disease, such as a cancer patient, a tumor patient, or a cancer and tumor patient. The subject may be predisposed to a risk of developing a disease such as a cancer or a tumor. The subject may be in remission from a disease, such as a cancer or a tumor. The subject may not have a cancer, may not have a tumor, or may not have a cancer nor a tumor. The subject may be healthy.

The term “tissue sample,” as used herein, generally refers to any tissue sample of a subject. For example, a tissue sample may be breast tissue or bladder tissue or other. A tissue sample may be a sample suspected or confirmed of having a disease such as a cancer or a tumor. A tissue sample may be a sample removed from a subject, such as a tissue biopsy, excised tissue, fine needle aspirate, tissue washing, cytology specimen, or combination thereof. A tissue sample may be an intact region of a patient's body receiving cancer therapy, such as radiation. A tissue sample may be a tumor in a patient's body. A tissue sample may comprise cancerous cells, tumor cells, non-cancerous cells, or a combination thereof. A tissue may comprise invasive cells, non-invasive cells, or a combination thereof. A tissue sample may be a breast tissue. A tissue sample may be a bladder tissue. A tissue sample may be a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “cell sample,” as used herein, generally refers to a population of cells. A cell sample may be a cell line, such as a cancer cell line (i.e. MCF-7 cells, MDA-MB-231 cells, SKBR3 cells, or BT-474 cells). A cell sample may be a primary cell culture sample, such as cells obtained from a subject. A cell sample may be a population of cells that may be isolated from a subject, such as a tissue biopsy, a cytology specimen, a blood sample or a fine needle aspirate (FNA) sample. A cell sample may be obtained from urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. A cell sample may comprise cancerous cells, non-cancerous cells, tumor cells, non-tumor cells, healthy cells, or any combination thereof. A cell sample may comprise invasive cells, non-invasive cells, or a combination thereof.

The term “invasive cell,” as used herein, generally refers to a cell that may leave its local environment and migrate to a different portion of a tissue, to a different organ or to a different part of the body of a subject. An invasive cell may secrete enzymes, such as matrix metalloproteinase, to break down matrix components to promote its migration. An invasive cell may migrate to and enter the vasculature or the lymphatics to travel throughout the body. A cell may be stratified or categorized as a non-invasive, weakly invasive, moderately invasive, or highly invasive cell. A highly invasive cell may have a higher probability than non-invasive, weakly invasive, or moderately invasive cells to migrate to a different organ or part of the body. For example, MDA-MB-231 breast cancer cells may be an example of highly invasive cells. In a cell invasion assay, transwell assay, or scratch wound assay, greater numbers of highly invasive cells may migrate than numbers of moderately invasive, weakly invasive, or non-invasive cells. A weakly invasive cell may have a small probability to migrate to a different organ or part of the body. For example, MCF-7 cancer cells may be an example of weakly invasive cells. In a cell invasion assay, transwell assay, or scratch wound assay, greater numbers of weakly invasive cells may migrate than non-invasive cells and highly invasive cells may migrate in higher numbers than both weakly invasive and non-invasive cells.

The term “likelihood of metastasis,” as used herein, generally refers to a probability of a cell to invade a different tissue space, such as a probability of a cancer cell to metastasize to a different part of an organ or a different organ. One cell may have a greater or lesser probability of invasion compared to a different cell. A cell may be non-invasive, generally lacking a probability to invade a different tissue space. A cell may be highly invasive, with a high probability to invade a different tissue space. Cells may be characterized by their likelihood of metastasis on a graded scale from non-invasive, weakly invasive, moderately invasive, to highly invasive. In vitro assays employed to determine a likelihood of metastasis of a cell or tissue may include a Boyden chamber assay, Matrigel invasion assay, a scratch-wound assay, a transwell assay, a cell invasion assay, a 3D protrusion assay, or others. Imaging assays employed to determine a likelihood of metastasis of a cell or tissue may include measuring intracellular calcium levels following an energy stimulus. Molecular assays employed to determine a likelihood of metastasis of a cell or tissue may include assaying for an expression level, a presence, or an absence of one or more biomarkers, such as a nucleotide or a protein. In some embodiments, the expression level, the presence, or the absence of the one or more biomarkers may indicate an invasive or non-invasive phenotype.

The term “risk of tumor or cancer occurrence,” as defined herein, generally refers to a risk or probability associate with the occurrence of a cancer in a subject. A risk of tumor or cancer occurrence can include a first occurrence of cancer in a subject or can include subsequent occurrences, such as a second, third, fourth, or subsequent occurrence. A risk of tumor or cancer occurrence can include a) a risk of developing the cancer for a first time, b) a risk of relapse or of developing the cancer again, c) a risk of developing the cancer in the future, d) a risk of being predisposed to developing the cancer in the subject's lifetime, or e) a risk of being predisposed to developing the cancer as an infant, adolescent, or adult. A risk of tumor or cancer occurrence or reccurrence can include a risk of the cancer becoming metastatic. A risk of tumor or cancer occurrence or reccurrence can include a risk of occurrence of a stage I cancer, a stage II cancer, a stage III cancer, or a stage IV cancer. Risk of tumor or cancer occurrence or reccurrence can include a risk for a blood cancer, tissue cancer (e.g., a tumor), or a cancer becoming metastatic to one or more organ sites from other sites.

The term “an effectiveness of a cancer or tumor treatment,” as defined herein, generally refers to an assessment or determination about whether a cancer or tumor treatment has achieved the results it is intended to achieve. For example, an effectiveness of a cancer treatment, such as administration of an anti-cancer drug, may be an assessment of the anti-cancer drug to reduce tumor or cancer cell invasiveness, to kill cancer or tumor cells, or to eliminate a cancer or tumor in a subject. A cancer or tumor treatment may include a surgery (i.e. surgical resection), a nutrition regime, a physical activity, radiation, chemotherapy, cell transplantation, blood fusion, or others.

The term “an effectiveness of a drug,” as defined herein, generally refers to an assessment or determination about whether a drug has achieved the results it is intended to achieve. For example, an effectiveness of an anti-cancer drug may be an assessment of the anti-cancer drug to reduce tumor or cancer cell invasiveness, to kill cancer or tumor cells, or to eliminate a cancer or tumor in a subject. An effectiveness of a drug may also include an assessment of the severity and number of side effects or conditions brought on by consuming the drug. The assessment or determination may be performed using methods as described herein.

The term “a longitudinal course of a cancer or tumor treatment regime,” as defined herein, generally refers to a time course over which a subject receives a cancer or tumor treatment regime. The cancer or tumor treatment regime can be an administration of a drug, such as an anti-cancer or anti-tumor drug to the subject over the time course. The time course may begin following a tumor or cancer diagnosis and continue until the subject is cancer or tumor free.

The term “diagnostic method,” as defined herein, generally refers to a method to diagnose a disease such as a cancer or a tumor. A diagnostic method may be performed using the methods as described herein. A diagnostic method may include a tissue biopsy, a fine needle aspiration, an endoscopy, a diagnostic imaging, a blood test, a genetic analysis, or combinations thereof.

The term “operation,” as defined herein, generally refers to a removal or a partial removal of a tissue, such as a cancerous tissue or a tumorous tissue, a resection or a partial resection of a tissue, such as a cancerous tissue or a tumorous tissue, or any combinations thereof.

A drug may be an anti-cancer drug, an anti-tumor drug, or an anti-cancer and anti-tumor drug. A drug may be used to reduce the invasiveness of cells, such as cancer cells. A drug may be used alone or in combination with another drug or treatment regime. A drug may be used to treat a subject suspected or confirmed to have a cancer, a tumor, or combination thereof. A drug may be used in a screening method to assess the effectiveness of the drug in reducing cell invasiveness. A drug may be administered to a subject, such as part of a cancer treatment regime. A drug may be administered to a cell sample, such as part of a screening method.

A drug may be a small molecule, a small interfering RNA (siRNA), a short hairpin RNA (shRNA), an antisense RNA (asRNA), a ribozyme, an antibody, an aptamer, or fragment thereof, or any combination thereof. A drug may be a tyrosine kinase inhibitor, an antibody, a small molecule, an alkylating agent, an antimetabolite, an antimicrobial, a plant alkaloid, a topoiosmerase inhibitor, any salt thereof, or any combination thereof. A drug may be a chemotherapeutic agent. A drug may be a preclinical stage drug. A preclinical stage drug may include a research drug. A preclinical stage drug may include any drug prior to filing of an Investigational New Drug Application (IND), with a regulatory agency, such as the Food and Drug Administration (FDA). A drug may be a clinical drug, such as a drug in clinical phases with a regulatory agency, such as the FDA. The drug may be a drug approved by a regulatory agency, such as the FDA.

A drug may comprise an siRNA drug. In some embodiments, the siRNA drug has at least 70% sequence homology to or at least 70% of the nucleobases or combination thereof of: GCAAAGAGCUGCUUUGUGUUUGGUA, UUUGCAAAGAGCUGCUUUGUGUUUGGU, or any fragment thereof, or any combination thereof. A drug may comprise an siRNA drug. In some embodiments, the siRNA drug has at least 80% sequence homology to or at least 80% of the nucleobases or combination thereof of: GCAAAGAGCUGCUUUGUGUUUGGUA, UUUGCAAAGAGCUGCUUUGUGUUUGGU, or any fragment thereof, or any combination thereof. A drug may comprise an siRNA drug. In some embodiments, the siRNA drug has at least 90% sequence homology to or at least 90% of the nucleobases or combination thereof of: GCAAAGAGCUGCUUUGUGUUUGGUA, UUUGCAAAGAGCUGCUUUGUGUUUGGU, or any fragment thereof, or any combination thereof. A drug may comprise an siRNA drug. In some embodiments, the siRNA drug has at least 95% sequence homology to or at least 95% of the nucleobases or combination thereof of: GCAAAGAGCUGCUUUGUGUUUGGUA, UUUGCAAAGAGCUGCUUUGUGUUUGGU, or any fragment thereof, or any combination thereof.

A drug may be an anti-cancer drug, an anti-tumor drug, or combination thereof. For example, the drug may be Abiraterone Acetate, Abitrexate (Methotrexate), Abraxane (Paclitaxel Albumin-stabilized Nanoparticle Formulation), Adcetris (Brentuximab Vedotin), Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, Becenum (Carmustine), Beleodaq (Belinostat), Belinostat, Bendamustine Hydrochloride, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, Campath (Alemtuzumab), Camptosar (Irinotecan Hydrochloride), Capecitabine, CAPDX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), Cobimetinib, Cometriq (Cabozantinib-S-Malate), Cosmegen (Dactinomycin), Cotellic (Cobimetinib), Crizotinib, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, 5-FU (Fluorouracil), Fareston (Toremifene), Farydak (Panobinostat), Faslodex (Fulvestrant), Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Margibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), Ofatumumab, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Revlimid (Lenalidomide), Rheumatrex (Methotrexate), Rituxan (Rituximab), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thioguanine, Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate) or any salt thereof or any combination thereof.

An expression level of a control may be a reference value obtained from a database. The expression level may be an average expression level. The average expression level may be for at least one of one or more oligonucleotide sequences. The average expression level may be for each of one or more oligonucleotide sequences. The average expression level for each of the one or more oligonucleotides sequences may be averaged from the individual expression levels of each sample in the database, such as 20 samples.

A database may be an online database. A database may comprise a user interface to interact with a user. A database may capture data, such as expression level data. A database may analyze data. A database may be configured for the user the select a control sample or to define a control sample. A user may query a database. A database may comprise a memory to store data, such as data obtained from assaying, such as expression level data.

A database may comprise expression level data obtained from at least 1, 2, 3, 4, 5, 6, 7, 8, 9, 10, 15, 20, 25, 30, 35, 40, 45, 50, 100, 150, 200, 250, 300, 500 noncancerous or non-tumor or noncancerous and non-tumor tissue samples. A database may comprise expression level data obtained from at least 1 noncancerous or non-tumor or noncancerous and non-tumor tissue sample. A database may comprise expression level data obtained from at least 2 noncancerous or non-tumor or noncancerous and non-tumor tissue samples. A database may comprise expression level data obtained from at least 5 noncancerous or non-tumor or noncancerous and non-tumor tissue samples. A database may comprise expression level data obtained from at least 10 noncancerous or non-tumor or noncancerous and non-tumor tissue samples. A database may comprise expression level data obtained from at least 20 noncancerous or non-tumor or noncancerous and non-tumor tissue samples. A database may comprise expression level data obtained from at least 50 noncancerous or non-tumor or noncancerous and non-tumor tissue samples. A database may comprise expression level data obtained from at least 100 noncancerous or non-tumor or noncancerous and non-tumor tissue samples. A database may comprise expression level data obtained from at least 200 noncancerous or non-tumor or noncancerous and non-tumor tissue samples. A database may comprise expression level data obtained from at least 500 noncancerous or non-tumor or noncancerous and non-tumor tissue samples. A database may comprise expression level data obtained from at least 1000 noncancerous or non-tumor or noncancerous and non-tumor tissue samples.

Cancer cells, tumor cells, or a combination thereof may be identified in a tissue sample. Identification may occur prior to assaying. Identification may occur after assaying. Identification may comprise staining the tissue sample for one or more cell surface markers, one or more intracellular markers, or a combination thereof. A tissue sample may be stained for 1, 2, 3, 4, 5, 6 or more cell surface markers, one or more intracellular markers, or a combination thereof.

A tissue sample may be enriched for cancer cells, tumor cells, or a combination thereof. Enriching a sample may include sorting for cancer cells, tumor cells, or a combination thereof. Sorting may include positive sorting, such as using a magnetic-activated cell sorting (MACS) column, wherein cancer cells or tumor cells bind a column based on cell surface marker expression. Sorting may include negative sorting, such as using a MACS column, wherein cancer cells or tumor cells are eluting through the column based on cell surface marker expression. Sorting may include sorting on a fluorescence activated cell sorting (FACS) machine based on cell surface marker expression. A tissue sample may be enriched for cancer cells, tumor cells, or a combination thereof by deleting other cell populations such as non-cancerous or non-tumorous cells. A tissue sample prepared for sorting may be stained for 1, 2, 3, 4, 5, 6, or more cell surface markers, one or more intracellular markers, or a combination thereof.

Cell surface markers may be fluorescently labeled, magnetically labeled, or not labeled. Cell surface markers may include cancer specific markers, tumor specific markers, markers that indicate high proliferation rates, markers that indicate metastasis or invasiveness or any combination thereof. Cell surface markers may include CD19, CD20, CD24, CD34, CD38, CD44, CD90, CD133, epithelial cell adhesion molecule (EpCAM), ATP-binding cassette transporter B5 (ABCBS), adhesion G-protein coupled receptor (GPR116), or any combination thereof. Cell surface markers may include CD44, GPR116, or a combination thereof.

The methods described herein, such as assaying and comparing, may be conducted prior to an operation on a tumor tissue or a cancer tissue of the subject, such as a tumor resection. The methods described herein may be conducted prior to the subject having a positive cancer diagnosis or a tumor diagnosis. The methods described herein may be conducted on a subject suspected of having a cancer or a tumor. The methods described herein may be conducted on a subject that has received a positive cancer diagnosis or a positive tumor diagnosis. The methods described herein may be conducted on a subject having received a prior treatment regime, wherein the prior treatment regime was ineffective in eliminating the cancer or tumor. A tissue sample may be obtained from a subject prior to performing the methods described herein. A tissue sample may be obtained during a biopsy, fine needle aspiration, blood sample, surgery resection, or any combination thereof.

The methods described herein may include at least one other diagnostic method. The methods described herein may include at least two other diagnostic methods. The at least one other diagnostic method may include a tissue biopsy, an endoscopy, a diagnostic imaging, a blood test, a genetic analysis, or combinations thereof.

Assaying a tissue sample of a subject may be performed at one or more time points. A separate tissue sample may be obtained from the subject for assaying at each of the one or more time points. Assaying at one or more time points may be performed on the same tissue sample. Assaying at one or more time points may provide an assessment of an effectiveness of a drug, a longitudinal course of a cancer or tumor treatment regime, or a combination thereof. At each of the one or more time points, a tissue sample may be compared to the same control. A tissue sample may be compared to a different control at each of the one or more time points. The one or more time points may be the same. The one or more time points may be different. The one or more time points may comprise at least one time point prior to a drug administration, at least one time point after a drug administration, at least one time point prior to a positive cancer diagnosis or a positive tumor diagnosis, at least one time point after a cancer remission diagnosis or tumor elimination diagnosis, at least one time point during a cancer treatment regime or a tumor treatment regime, or a combination thereof.

One or more oligonucleotide sequences may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of a REST-003 or fragment thereof. One or more oligonucleotide sequences may have at least 60% homology to or at least 60% of the nucleobases or any combination thereof of at least a portion of a REST-003 or fragment thereof. One or more oligonucleotide sequences may have at least 65% homology to or at least 65% of the nucleobases or any combination thereof of at least a portion of a REST-003 or fragment thereof. One or more oligonucleotide sequences may have at least 70% homology to or at least 70% of the nucleobases or any combination thereof of at least a portion of a REST-003 or fragment thereof. One or more oligonucleotide sequences may have at least 75% homology to or at least 75% of the nucleobases or any combination thereof of at least a portion of a REST-003 or fragment thereof. One or more oligonucleotide sequences may have at least 80% homology to or at least 80% of the nucleobases or any combination thereof of at least a portion of a REST-003 or fragment thereof. One or more oligonucleotide sequences may have at least 85% homology to or at least 85% of the nucleobases or any combination thereof of at least a portion of a REST-003 or fragment thereof. One or more oligonucleotide sequences may have at least 90% homology to or at least 90% of the nucleobases or any combination thereof of at least a portion of a REST-003 or fragment thereof. One or more oligonucleotide sequences may have at least 95% homology to or at least 95% of the nucleobases or any combination thereof of at least a portion of a REST-003 or fragment thereof.

One or more oligonucleotide sequences may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of a REST-003-mediated oligonucleotide sequence. One or more oligonucleotide sequences may have at least 60% homology to or at least 60% of the nucleobases or any combination thereof of at least a portion of a REST-003-mediated oligonucleotide sequence. One or more oligonucleotide sequences may have at least 65% homology to or at least 65% of the nucleobases or any combination thereof of at least a portion of a REST-003 or fragment thereof. One or more oligonucleotide sequences may have at least 70% homology to or at least 70% of the nucleobases or any combination thereof of at least a portion of a REST-003-mediated oligonucleotide sequence. One or more oligonucleotide sequences may have at least 75% homology to or at least 75% of the nucleobases or any combination thereof of at least a portion of a REST-003-mediated oligonucleotide sequence. One or more oligonucleotide sequences may have at least 80% homology to or at least 80% of the nucleobases or any combination thereof of at least a portion of a REST-003-mediated oligonucleotide sequence. One or more oligonucleotide sequences may have at least 85% homology to or at least 85% of the nucleobases or any combination thereof of at least a portion of a REST-003-mediated oligonucleotide sequence. One or more oligonucleotide sequences may have at least 90% homology to or at least 90% of the nucleobases or any combination thereof of at least a portion of a REST-003-mediated oligonucleotide sequence. One or more oligonucleotide sequences may have at least 95% homology to or at least 95% of the nucleobases or any combination thereof of at least a portion of a REST-003-mediated oligonucleotide sequence.

A marker may be a primer for a REST-003 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 1, AGTGTCGGGGCGACTCCCG. A marker may have at least 60% homology to or at least 60% of the nucleobases or any combination thereof of at least a portion of SEQ. 1. A marker may have at least 65% homology to or at least 65% of the nucleobases or any combination thereof of at least a portion of SEQ. 1. A marker may have at least 70% homology to or at least 70% of the nucleobases or any combination thereof of at least a portion of SEQ. 1. A marker may have at least 75% homology to or at least 75% of the nucleobases or any combination thereof of at least a portion of SEQ. 1. A marker may have at least 80% homology to or at least 80% of the nucleobases or any combination thereof of at least a portion of SEQ. 1. A marker may have at least 85% homology to or at least 85% of the nucleobases or any combination thereof of at least a portion of SEQ. 1. A marker may have at least 90% homology to or at least 90% of the nucleobases or any combination thereof of at least a portion of SEQ. 1. A marker may have at least 95% homology to or at least 95% of the nucleobases or any combination thereof of at least a portion of SEQ. 1.

A marker may be a primer for a REST-003 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 2, GGCATTCCTAACTGAAATAGG. A marker may have at least 60% homology to or at least 60% of the nucleobases or any combination thereof of at least a portion of SEQ. 2. A marker may have at least 65% homology to or at least 65% of the nucleobases or any combination thereof of at least a portion of SEQ. 2. A marker may have at least 70% homology to or at least 70% of the nucleobases or any combination thereof of at least a portion of SEQ. 2. A marker may have at least 75% homology to or at least 75% of the nucleobases or any combination thereof of at least a portion of SEQ. 2. A marker may have at least 80% homology to or at least 80% of the nucleobases or any combination thereof of at least a portion of SEQ. 2. A marker may have at least 85% homology to or at least 85% of the nucleobases or any combination thereof of at least a portion of SEQ. 2. A marker may have at least 90% homology to or at least 90% of the nucleobases or any combination thereof of at least a portion of SEQ. 2. A marker may have at least 95% homology to or at least 95% of the nucleobases or any combination thereof of at least a portion of SEQ. 2.

A marker may be a primer for a SRRM3_1 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 3, TGGTGAAGCGCGCGCACCGCGAGATCC. A marker may have at least 60% homology to or at least 60% of the nucleobases or any combination thereof of at least a portion of SEQ. 3. A marker may have at least 65% homology to or at least 65% of the nucleobases or any combination thereof of at least a portion of SEQ. 3. A marker may have at least 70% homology to or at least 70% of the nucleobases or any combination thereof of at least a portion of SEQ. 3. A marker may have at least 75% homology to or at least 75% of the nucleobases or any combination thereof of at least a portion of SEQ. 3. A marker may have at least 80% homology to or at least 80% of the nucleobases or any combination thereof of at least a portion of SEQ. 3. A marker may have at least 85% homology to or at least 85% of the nucleobases or any combination thereof of at least a portion of SEQ. 3. A marker may have at least 90% homology to or at least 90% of the nucleobases or any combination thereof of at least a portion of SEQ. 3. A marker may have at least 95% homology to or at least 95% of the nucleobases or any combination thereof of at least a portion of SEQ. 3.

A marker may be a primer for a SRRM3_1 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 4, GAATGTCCCCACTTTCTGCCGAATC. A marker may have at least 60% homology to or at least 60% of the nucleobases or any combination thereof of at least a portion of SEQ. 4. A marker may have at least 65% homology to or at least 65% of the nucleobases or any combination thereof of at least a portion of SEQ. 4. A marker may have at least 70% homology to or at least 70% of the nucleobases or any combination thereof of at least a portion of SEQ. 4. A marker may have at least 75% homology to or at least 75% of the nucleobases or any combination thereof of at least a portion of SEQ. 4. A marker may have at least 80% homology to or at least 80% of the nucleobases or any combination thereof of at least a portion of SEQ. 4. A marker may have at least 85% homology to or at least 85% of the nucleobases or any combination thereof of at least a portion of SEQ. 4. A marker may have at least 90% homology to or at least 90% of the nucleobases or any combination thereof of at least a portion of SEQ. 4. A marker may have at least 95% homology to or at least 95% of the nucleobases or any combination thereof of at least a portion of SEQ. 4.

A marker may be a primer for a SRRM3_2 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 5, TCCTGGAGCTCCAGCCGCTCGCCC. A marker may have at least 60% homology to or at least 60% of the nucleobases or any combination thereof of at least a portion of SEQ. 5. A marker may have at least 65% homology to or at least 65% of the nucleobases or any combination thereof of at least a portion of SEQ. 5. A marker may have at least 70% homology to or at least 70% of the nucleobases or any combination thereof of at least a portion of SEQ. 5. A marker may have at least 75% homology to or at least 75% of the nucleobases or any combination thereof of at least a portion of SEQ. 5. A marker may have at least 80% homology to or at least 80% of the nucleobases or any combination thereof of at least a portion of SEQ. 5. A marker may have at least 85% homology to or at least 85% of the nucleobases or any combination thereof of at least a portion of SEQ. 5. A marker may have at least 90% homology to or at least 90% of the nucleobases or any combination thereof of at least a portion of SEQ. 5. A marker may have at least 95% homology to or at least 95% of the nucleobases or any combination thereof of at least a portion of SEQ. 5.

A marker may be a primer for a SRRM3_2 or fragment thereof. For example, a marker may have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% homology to or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the nucleobases or any combination thereof of at least a portion of oligonucleotide sequence, SEQ. 6, CTCAGAGTGCCTTGCGCGGCCCTCG. A marker may have at least 60% homology to or at least 60% of the nucleobases or any combination thereof of at least a portion of SEQ. 6. A marker may have at least 65% homology to or at least 65% of the nucleobases or any combination thereof of at least a portion of SEQ. 6. A marker may have at least 70% homology to or at least 70% of the nucleobases or any combination thereof of at least a portion of SEQ. 6. A marker may have at least 75% homology to or at least 75% of the nucleobases or any combination thereof of at least a portion of SEQ. 6. A marker may have at least 80% homology to or at least 80% of the nucleobases or any combination thereof of at least a portion of SEQ. 6. A marker may have at least 85% homology to or at least 85% of the nucleobases or any combination thereof of at least a portion of SEQ. 6. A marker may have at least 90% homology to or at least 90% of the nucleobases or any combination thereof of at least a portion of SEQ. 6. A marker may have at least 95% homology to or at least 95% of the nucleobases or any combination thereof of at least a portion of SEQ. 6.

One or more REST-003-mediated oligonucleotide sequences may comprise PLEC, MAGED1, SYK, STK35, ANXA10, EHF, SLC35B2, CUL4A, EPCAM, MTMR4, or combinations thereof. One or more REST-003-mediated oligonucleotide sequences may comprise PLEC. One or more REST-003-mediated oligonucleotide sequences may comprise MAGED1. One or more REST-003-mediated oligonucleotide sequences may comprise SYK. One or more REST-003-mediated oligonucleotide sequences may comprise STK35. One or more REST-003-mediated oligonucleotide sequences may comprise ANXA10. One or more REST-003-mediated oligonucleotide sequences may comprise EHF. One or more REST-003-mediated oligonucleotide sequences may comprise SLC35B2. One or more REST-003-mediated oligonucleotide sequences may comprise CUL4A. One or more REST-003-mediated oligonucleotide sequences may comprise EPCAM. One or more REST-003-mediated oligonucleotide sequences may comprise MTMR4.

One or more REST-003-mediated oligonucleotide sequences may comprise IFNL1, CXCL10, IFNB1, CXCL11, CCR1, GBP5, APOL3, GBP4, C1S, CASP1, XAF1, CCL5, IDO1, IRG1, GBP1, TNFSF10, CD274, RTP4, IFIT2, TFPI2, APOL1, GBP1P1, BST2, IFIT3, TGFBI, TRIM22, PSAT1, RSAD2, CEACAM1, GBP2, TMEM171, IL8, TLR3, CBX1, OASL, SERPINE1, MMP13, IL1B, HERC5, FNDC3A, CMPK2, ARL6IP1, PGAM1, TAP1, PMAIP1, IL6, or combinations thereof. One or more REST-003-mediated oligonucleotide sequences may comprise IFNL1. One or more REST-003-mediated oligonucleotide sequences may comprise CXCL10. One or more REST-003-mediated oligonucleotide sequences may comprise IFNB1. One or more REST-003-mediated oligonucleotide sequences may comprise CXCL11. One or more REST-003-mediated oligonucleotide sequences may comprise CCR1. One or more REST-003-mediated oligonucleotide sequences may comprise GBP5. One or more REST-003-mediated oligonucleotide sequences may comprise APOL3. One or more REST-003-mediated oligonucleotide sequences may comprise GBP4. One or more REST-003-mediated oligonucleotide sequences may comprise C1S. One or more REST-003-mediated oligonucleotide sequences may comprise CASP1. One or more REST-003-mediated oligonucleotide sequences may comprise XAF1. One or more REST-003-mediated oligonucleotide sequences may comprise CCL5. One or more REST-003-mediated oligonucleotide sequences may comprise IDO1. One or more REST-003-mediated oligonucleotide sequences may comprise IRG1. One or more REST-003-mediated oligonucleotide sequences may comprise GBP1. One or more REST-003-mediated oligonucleotide sequences may comprise TNFSF10. One or more REST-003-mediated oligonucleotide sequences may comprise CD274. One or more REST-003-mediated oligonucleotide sequences may comprise RTP4. One or more REST-003-mediated oligonucleotide sequences may comprise IFIT2. One or more REST-003-mediated oligonucleotide sequences may comprise TFPI2. One or more REST-003-mediated oligonucleotide sequences may comprise APOL1. One or more REST-003-mediated oligonucleotide sequences may comprise GBP1P1. One or more REST-003-mediated oligonucleotide sequences may comprise BST2. One or more REST-003-mediated oligonucleotide sequences may comprise IFIT3. One or more REST-003-mediated oligonucleotide sequences may comprise TGFBI. One or more REST-003-mediated oligonucleotide sequences may comprise TRIM22. One or more REST-003-mediated oligonucleotide sequences may comprise PSAT1. One or more REST-003-mediated oligonucleotide sequences may comprise RSAD2. One or more REST-003-mediated oligonucleotide sequences may comprise CEACAM1. One or more REST-003-mediated oligonucleotide sequences may comprise GBP2. One or more REST-003-mediated oligonucleotide sequences may comprise TMEM171. One or more REST-003-mediated oligonucleotide sequences may comprise IL8. One or more REST-003-mediated oligonucleotide sequences may comprise TLR3. One or more REST-003-mediated oligonucleotide sequences may comprise CBX1. One or more REST-003-mediated oligonucleotide sequences may comprise OASL. One or more REST-003-mediated oligonucleotide sequences may comprise SERPINE1. One or more REST-003-mediated oligonucleotide sequences may comprise MMP13. One or more REST-003-mediated oligonucleotide sequences may comprise IL1B. One or more REST-003-mediated oligonucleotide sequences may comprise HERC5. One or more REST-003-mediated oligonucleotide sequences may comprise FNDC3A. One or more REST-003-mediated oligonucleotide sequences may comprise CMPK2. One or more REST-003-mediated oligonucleotide sequences may comprise ARL6IP1. One or more REST-003-mediated oligonucleotide sequences may comprise PGAM1. One or more REST-003-mediated oligonucleotide sequences may comprise TAP1. One or more REST-003-mediated oligonucleotide sequences may comprise PMAIP1. One or more REST-003-mediated oligonucleotide sequences may comprise IL6.

The one or more REST-003-mediated oligonucleotide sequences or fragments thereof may comprise less than about 10 different genes. The one or more REST-003-mediated oligonucleotide sequences or fragments thereof may comprise less than about 20 different genes. The one or more REST-003-mediated oligonucleotide sequences or fragments thereof may comprise less than about 30 different genes. The one or more REST-003-mediated oligonucleotide sequences or fragments thereof may comprise less than about 40 different genes. The one or more REST-003-mediated oligonucleotide sequences or fragments thereof may comprise less than about 50 different genes. The one or more REST-003-mediated oligonucleotide sequences or fragments thereof may comprise less than about 60 different genes. The one or more REST-003-mediated oligonucleotide sequences or fragments thereof may comprise less than about 70 different genes. The one or more REST-003-mediated oligonucleotide sequences or fragments thereof may comprise less than about 80 different genes. The one or more REST-003-mediated oligonucleotide sequences or fragments thereof may comprise less than about 90 different genes. The one or more REST-003-mediated oligonucleotide sequences or fragments thereof may comprise less than about 100 different genes.

The one or more REST-003-mediated oligonucleotide sequences or fragments thereof may comprise from 1 to 20 different genes. The one or more REST-003-mediated oligonucleotide sequences or fragments thereof may comprise from 1 to 30 different genes. The one or more REST-003-mediated oligonucleotide sequences or fragments thereof may comprise from 1 to 40 different genes. The one or more REST-003-mediated oligonucleotide sequences or fragments thereof may comprise from 1 to 50 different genes. The one or more REST-003-mediated oligonucleotide sequences or fragments thereof may comprise from 10 to 30 different genes. The one or more REST-003-mediated oligonucleotide sequences or fragments thereof may comprise from 10 to 40 different genes. The one or more REST-003-mediated oligonucleotide sequences or fragments thereof may comprise from 10 to 50 different genes. The one or more REST-003-mediated oligonucleotide sequences or fragments thereof may comprise from 10 to 60 different genes. The one or more REST-003-mediated oligonucleotide sequences or fragments thereof may comprise from 10 to 70 different genes. The one or more REST-003-mediated oligonucleotide sequences or fragments thereof may comprise from 10 to 80 different genes.

The one or more REST-003-mediated oligonucleotide sequences or fragments thereof may comprise from 20 to 40 different genes. The one or more REST-003-mediated oligonucleotide sequences or fragments thereof may comprise from 20 to 50 different genes. The one or more REST-003-mediated oligonucleotide sequences or fragments thereof may comprise from 20 to 60 different genes. The one or more REST-003-mediated oligonucleotide sequences or fragments thereof may comprise from 20 to 70 different genes. The one or more REST-003-mediated oligonucleotide sequences or fragments thereof may comprise from 20 to 80 different genes. The one or more REST-003-mediated oligonucleotide sequences or fragments thereof may comprise from 20 to 90 different genes. The one or more REST-003-mediated oligonucleotide sequences or fragments thereof may comprise from 20 to 100 different genes.

A length of one or more oligonucleotide sequences may be from 70 to 150 nucleobases. A length of one or more oligonucleotide sequences may be from 70 to 140 nucleobases. A length of one or more oligonucleotide sequences may be from 80 to 140 nucleobases. A length of one or more oligonucleotide sequences may be from 70 to 90 nucleobases. A length of one or more oligonucleotide sequences may be from 30 to 150 nucleobases. A length of one or more oligonucleotide sequences may be from 30 to 200 nucleobases. A length of one or more oligonucleotide sequences may be from 70 to 200 nucleobases. A length of one or more oligonucleotide sequences may be less than 200 nucleobases. A length of one or more oligonucleotide sequences may be less than 175 nucleobases. A length of one or more oligonucleotide sequences may be less than 150 nucleobases. A length of one or more oligonucleotide sequences may be less than 140 nucleobases. A length of one or more oligonucleotide sequences may be less than 125 nucleobases. A length of one or more oligonucleotide sequences may be less than 100 nucleobases. A length of one or more oligonucleotide sequences may be less than 90 nucleobases. A length of one or more oligonucleotide sequences may be less than 80 nucleobases.

A length of each of one or more oligonucleotide sequences may be from 70 to 150 nucleobases. A length of each of one or more oligonucleotide sequences may be from 70 to 140 nucleobases. A length of each of one or more oligonucleotide sequences may be from 80 to 140 nucleobases. A length of each of one or more oligonucleotide sequences may be from 70 to 90 nucleobases. A length of each of one or more oligonucleotide sequences may be from 30 to 150 nucleobases. A length of each of one or more oligonucleotide sequences may be from 30 to 200 nucleobases. A length of each of one or more oligonucleotide sequences may be from 70 to 200 nucleobases. A length of each of one or more oligonucleotide sequences may be less than 200 nucleobases. A length of each of one or more oligonucleotide sequences may be less than 175 nucleobases. A length of each of one or more oligonucleotide sequences may be less than 150 nucleobases. A length of each of one or more oligonucleotide sequences may be less than 140 nucleobases. A length of each of one or more oligonucleotide sequences may be less than 125 nucleobases. A length of each of one or more oligonucleotide sequences may be less than 100 nucleobases. A length of each of one or more oligonucleotide sequences may be less than 90 nucleobases. A length of each of one or more oligonucleotide sequences may be less than 80 nucleobases.

A cancer or tumor as disclosed herein can include breast cancer or bladder cancer. Types of cancer include adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, Waldenstrom's macroglobulinemia, or any combination thereof.

Assaying may include assaying for an expression level of one or more oligonucleotide sequences, assaying for a presence or absence of one or more oligonucleotide sequences, or a combination thereof. Assaying may be used alone or in combination with other diagnostic or screening methods or criteria.

The one or more oligonucleotide sequences may be known sequences or unknown sequences. The one or more oligonucleotide sequences may be known or unknown functions. The one or more oligonucleotide sequences may be a biomarker. The one or more oligonucleotide sequences may be associated with a disease, such as cancer. The one or more oligonucleotide sequences may be associated with a likelihood of metastasis of the cell sample. The one or more oligonucleotide sequences may be a known biomarker for a disease such as cancer. The one or more oligonucleotide sequences may be a known biomarker for indicating likelihood of metastasis of a cell, such as highly invasive or non-invasive. The one or more oligonucleotide sequences may be a known biomarker for a risk of occurrence or recurrence of cancer. The one or more oligonucleotide sequences may be a new biomarker that may indicate (a) a type of cancer, (b) a likelihood of metastasis, (c) a risk for tumor or cancer occurrence or reccurrence, (d) an effectiveness of a cancer or tumor treatment, (e) an effectiveness of a drug, (f) a longitudinal course of a cancer or tumor treatment regime or (g) any combination thereof.

The presence, absence, or expression level of one or more oligonucleotide sequences may inform a disease diagnosis (such as a cancer diagnosis), a progress report (such as a report of disease remission or progression or treatment efficacy), a treatment regime (such as changing or keeping a particular treatment regime), a level of effectiveness of a drug alone (such as “effective” or “non-effective” in curing the disease), predicting a patient outcome (such as “in remission” or “not in remission”) or any combination thereof.

General methods for determining expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immuno-histochemistry, blotting, sequencing, RNA sequencing, DNA sequencing (e.g., sequencing of complementary deoxyribonucleic acid (cDNA) obtained from RNA); next generation (Next-Gen) sequencing, nanopore sequencing, pyrosequencing, Nanostring sequencing, microarrays, reverse transcriptase polymerase chain reaction (RT-PCR), quantitative RT-PCR (qRT-PCR), real-time reverse transcriptase PCR (RT-rtPCR), nested PCR, or high-throughput RNA sequencing (RNA-seq), or combinations thereof. Gene expression product levels may be normalized to an internal standard such as total messenger ribonucleic acid (mRNA) or the expression level of a particular gene.

Next-generation sequencing may also be known as high-throughput sequencing or massively parallel sequencing, including Illumina sequencing, Roche 454 sequencing, ion torrent: proton sequencing, SOLiD sequencing and others. These sequencing methods sequence oligonucleotide sequences faster and inexpensively compared with Sanger sequencing. Nanopore sequencing may determine the order in which oligonucleotides occur on a strand of DNA by immersing a nanopore in a conducting fluid and applying a potential voltage across it. SAGE is a technique that may produce a list of short oligonucleotide sequence tags and the number of times each short oligonucleotide sequence tag is observed in a sample.

Assaying may comprise array hybridization, a serial analysis of gene expression (SAGE), an enzyme linked immunoabsorbance assay, a mass spectrometry, an immuno-histochemistry, a blotting, a nucleic acid sequencing, nucleic acid amplification, or any combination thereof. Assaying may include using markers that are selected for the one or more oligonucleotide sequences. Assaying may comprise an array hybridization. Assaying may comprise SAGE. Assaying may comprise an enzyme linked immunoabsorbance assay. Assaying may comprise a mass spectrometry. Assaying may comprise an immuno-histochemistry assay. Assaying may comprise blotting. Assaying may comprise nucleic acid sequencing. Assaying may comprise nucleic acid amplification.

An expression level of the one or more REST-003-medicated oligonucleotide sequences or fragments thereof may be at least about 0.01%, 0.1%, 0.5%, 1%, 2%, 3%, 4%, 6%, 7%, 8%, 9%, 10%, 11%, 12%, 13%, 14%, 15%, 20%, 25%, 30%, 35%, 40%, 45% or 50% higher than an expression level of a control. An expression level of the one or more REST-003-mediated oligonucleotide sequences or fragments thereof may be at least about 0.01% higher than an expression level of a control. An expression level of the one or more REST-003-mediated oligonucleotide sequences or fragments thereof may be at least about 0.1% higher than an expression level of a control. An expression level of the one or more REST-003-mediated oligonucleotide sequences or fragments thereof may be at least about 0.5% higher than an expression level of a control. An expression level of the one or more REST-003-mediated oligonucleotide sequences or fragments thereof may be at least about 1% higher than an expression level of a control. An expression level of the one or more REST-003-mediated oligonucleotide sequences or fragments thereof may be at least about 2% higher than an expression level of a control. An expression level of the one or more REST-003-mediated oligonucleotide sequences or fragments thereof may be at least about 3% higher than an expression level of a control. An expression level of the one or more REST-003-mediated oligonucleotide sequences or fragments thereof may be at least about 4% higher than an expression level of a control. An expression level of the one or more REST-003-mediated oligonucleotide sequences or fragments thereof may be at least about 5% higher than an expression level of a control. An expression level of the one or more REST-003-mediated oligonucleotide sequences or fragments thereof may be at least about 10% higher than an expression level of a control.

An expression level of the one or more REST-003-mediated oligonucleotide sequences or fragments thereof may be at least about 0.01%, 0.1%, 0.5%, 1%, 2%, 3%, 4%, 5%, 6%, 8%, 9%, 10%, 11%, 12%, 13%, 14%, 15%, 20%, 25%, 30%, 35%, 40%, 45% or 50% lower than an expression level of a control. An expression level of the one or more REST-003-mediated oligonucleotide sequences or fragments thereof may be at least about 0.01% lower than an expression level of a control. An expression level of the one or more REST-003-mediated oligonucleotide sequences or fragments thereof may be at least about 0.1% lower than an expression level of a control. An expression level of the one or more REST-003-mediated oligonucleotide sequences or fragments thereof may be at least about 0.5% lower than an expression level of a control. An expression level of the one or more REST-003-mediated oligonucleotide sequences or fragments thereof may be at least about 1% lower than an expression level of a control. An expression level of the one or more REST-003-mediated oligonucleotide sequences or fragments thereof may be at least about 2% lower than an expression level of a control. An expression level of the one or more REST-003-mediated oligonucleotide sequences or fragments thereof may be at least about 3% lower than an expression level of a control, An expression level of the one or more REST-003-mediated oligonucleotide sequences or fragments thereof may be at least about 4% lower than an expression level of a control. An expression level of the one or more REST-003-mediated oligonucleotide sequences or fragments thereof may be at least about 5% lower than an expression level of a control. An expression level of the one or more REST-003-mediated oligonucleotide sequences or fragments thereof may be at least about 10% lower than an expression level of a control.

An expression level of the one or more REST-003-mediated oligonucleotide sequences or fragments thereof may be at least about 2, 3, 4, 5, 6, 7, 8, 9, 10 fold difference higher than an expression level of a control. An expression level of the one or more REST-003-mediated oligonucleotide sequences or fragments thereof may be at least about 2 fold different higher than an expression level of a control, An expression level of the one or more REST-003-mediated oligonucleotide sequences or fragments thereof may be at least about 3 fold different higher than an expression level of a control. An expression level of the one or more REST-003-mediated oligonucleotide sequences or fragments thereof may be at least about 4 fold different higher than an expression level of a control, An expression level of the one or more REST-003-mediated oligonucleotide sequences or fragments thereof may be at least about 5 fold different higher than an expression level of a control. An expression level of the one or more REST-003-mediated oligonucleotide sequences or fragments thereof may be at least about 6 fold different higher than an expression level of a control. An expression level of the one or more REST-003-mediated oligonucleotide sequences or fragments thereof may be at least about 7 fold different higher than an expression level of a control. An expression level of the one or more REST-003-mediated oligonucleotide sequences or fragments thereof may be at least about 8 fold different higher than an expression level of a control. An expression level of the one or more REST-003-mediated oligonucleotide sequences or fragments thereof may be at least about 9 fold different higher than an expression level of a control, An expression level of the one or more REST-003-mediated oligonucleotide sequences or fragments thereof may be at least about 10 fold different higher than an expression level of a control.

An expression level of the one or more REST-003-mediated oligonucleotide sequences or fragments thereof may be at least about 2, 3, 4, 5, 6, 7, 8, 9, 10 fold difference lower than an expression level of a control. An expression level of the one or more REST-003-mediated oligonucleotide sequences or fragments thereof may be at least about 2 fold different lower than an expression level of a control. An expression level of the one or more REST-003-mediated oligonucleotide sequences or fragments thereof may be at least about 3 fold different lower than an expression level of a control. An expression level of the one or more REST-003-mediated oligonucleotide sequences or fragments thereof may be at least about 4 fold different lower than an expression level of a control. An expression level of the one or more REST-003-mediated oligonucleotide sequences or fragments thereof may be at least about 5 fold different lower than an expression level of a control. An expression level of the one or more REST-003-mediated oligonucleotide sequences or fragments thereof may be at least about 6 fold different lower than an expression level of a control. An expression level of the one or more REST-003-mediated oligonucleotide sequences or fragments thereof may be at least about 7 fold different lower than an expression level of a control. An expression level of the one or more REST-003-mediated oligonucleotide sequences or fragments thereof may be at least about 8 fold different lower than an expression level of a control. An expression level of the one or more REST-003-mediated oligonucleotide sequences or fragments thereof may be at least about 9 fold different lower than an expression level of a control. An expression level of the one or more REST-003-mediated oligonucleotide sequences or fragments thereof may be at least about 10 fold different lower than an expression level of a control.

A molecular pathway that controls cancer invasion was recently discovered. The invention describes methods for targeting and inhibiting that pathway, thereby limiting tumor invasion and metastasis. The pathway relates to a gene called RE1-Silencing Transcription factor (REST), also known as Neuron-Restrictive Silencer Factor (NRSF). REST has a well-established role in regulating (silencing) gene transcription during neuronal development, and its dysfunction has been recently been implicated in cancer. Studies have established a strong correlation between cancer invasiveness and loss of REST expression. This relationship is explored in more detail, which led to the discovery of new diagnostic and therapeutic targets. The methods of the present invention are particularly suited to detecting tumor invasion and metastasis in cancer, including breast cancer and bladder cancer.

The REST gene has multiple exons (DNA coding sequences) and undergoes a process called alternative splicing, in which the gene codes for multiple RNA transcripts and proteins. REST is known to produce four alternative splicing transcripts (RNAs), REST-001, REST-002, REST-003, and REST-004, though little is known about their function. REST-003 positively correlates with cancer invasiveness. REST-003 does not code for protein but is rather processed into several non-coding RNAs that regulate tumor invasion. Furthermore, REST-003 processing is regulated by a previously uncharacterized protein, SRRM3, whose expression is mediated by REST gene expression levels. A decrease in REST expression leads to higher SRRM3 levels, causing increased REST-003 expression and invasiveness.

At least 8 small non-coding RNAs are derived from REST-003 were discovered, which include both sense (S) and antisense (AS) sequences. Three have a direct role in promoting invasiveness, while the other 5 could potentially code for modified proteins and/or produce other non-coding RNAs that inhibit invasiveness, similar to the REST protein. The 3 non-coding RNAs that directly affect invasiveness have lengths of ˜75, ˜87, and ˜135 nucleotides (nt). Higher levels of the 75-nt AS, 87-nt S, 87-nt AS, and 135-nt AS strands, as well as lower levels of 135-nt S strand, are all positive indicators of invasiveness.

The findings include that several molecules can be targeted to limit tumor invasion and metastasis. These include: (1) REST-003 RNA—Decreasing expression would limit invasion. (2) SRRM3 mRNA or protein—Decreasing expression would limit invasion. (3) Small non-coding RNAs derived from REST-003—Decreasing expression of the 75-nt AS, 87-nt S, 87-nt AS, and/or 135-nt AS strands (or their targets) would limit invasion. Increasing expression of the 135-nt S strand (or its targets) would also limit invasion.

Several types of drugs could be used to target and inhibit these molecules. These include but are not limited to small molecules, small interfering RNA (siRNA), short hairpin RNA (shRNA), antisense RNA (asRNA), ribozymes, antibodies, and aptamers.

This invention provides methods for assessing whether a tumor is invasive or has the potential to invade and metastasize. The methods comprise analyzing the tumor, including cells thereof, for certain biomarkers that correlate with invasiveness. The biomarkers are generally derived from or relate to a gene called RE1-Silencing Transcription factor (REST), also known as Neuron-Restrictive Silencer Factor (NRSF). REST has a well-established role in regulating (silencing) gene transcription during neuronal development, and its dysfunction has been recently been implicated in cancer. Studies have established a strong correlation between cancer invasiveness and loss of REST expression. This relationship was explored in more detail, which led to the discovery of new biomarkers of invasiveness. The findings are applicable to many cancer types.

The REST gene has multiple exons (DNA coding sequences) and undergoes a process called alternative splicing, in which the gene codes for multiple RNA transcripts and proteins. REST is known to produce four alternative splicing transcripts (RNAs), REST-001, REST-002, REST-003, and REST-004, though little is known about their function. One aspect of the present invention is the finding that REST-003 positively correlates with cancer invasiveness. REST-003 does not code for protein but is rather processed into several non-coding RNAs that regulate tumor invasion. Furthermore, REST-003 processing is regulated by a previously uncharacterized protein, SRRM3, whose expression is mediated by REST gene expression levels. A decrease in REST expression leads to higher SRRM3 levels, causing increased REST-003 expression and invasiveness.

At least 8 small non-coding RNAs were discovered to be derived from REST-003, which include both sense (S) and antisense (AS) sequences. Three have a direct role in promoting invasiveness, while the other 5 could potentially code for modified proteins and/or produce other non-coding RNAs that inhibit invasiveness, similar to the REST protein. The 3 non-coding RNAs that directly affect invasiveness have lengths of ˜75, ˜87, and ˜135 nucleotides (nt). Higher levels of the 75-nt AS, 87-nt S, 87-nt AS, and 135-nt AS strands, as well as lower levels of 135-nt S strand, are all positive indicators of invasiveness.

REST-003 mediates expression of more than 50 target genes, some of which it upregulates and others of which it downregulates. Increased REST-003 expression leads to upregulation of 10 genes (indicating increased invasiveness), while decreased REST-003 expression leads upregulation of 46 genes (indicating decreased invasiveness). Detecting differential expression of these 56 genes in a biopsied tumor could serve as an indicator of cancer invasion.

Another aspect of the present invention comprises several biomarkers of tumor invasiveness. Specifically, an assay or assays for one or more of the following markers could indicate invasive cancer that is likely to metastasize: (1) High REST-003 RNA expression levels. (2) Altered SRRM3 mRNA and/or protein levels. (3) High expression of REST-003 fragment that produces one or more, or two or more, or three or more of the 75-nt AS, 87-nt S, 87-nt AS, and 135-nt AS strands. (4) Low expression of REST-003 fragment that produces the 135-nt S strand. (5) High expression of the one or more of 10 genes associated with high REST-003 levels: PLEC, MAGED1, SYK, STK35, ANXA10, EHF, SLC35B2, CUL4A, EPCAM, and MTMR4. (6) Low expression of one or more of the 46 genes associated with high REST-003 levels: IFNL1, CXCL10, IFNB1, CXCL11, CCR1, GBP5, APOL3, GBP4, C1S, CASP1, XAF1, CCL5, IDO1, IRG1, GBP1, TNFSF10, CD274, RTP4, IFIT2, TFPI2, APOL1, GBP1P1, BST2, IFIT3, TGFBI, TRIM22, PSAT1, RSAD2, CEACAM1, GBP2, TMEM171, IL8, TLR3, CBX1, OASL, SERPINE1, MMP13, IL1B, HERC5, FNDC3A, CMPK2, ARL6IP1, PGAM1, TAP1, PMAIP1, and IL6.

Several common laboratory techniques may use to assess these biomarkers. These include but are not limited to microarrays, reverse transcriptase polymerase chain reaction (RT-PCR), quantitative RT-PCR (qRT-PCR), real-time reverse transcriptase PCR (RT-rtPCR), nested PCR, and high-throughput RNA sequencing (RNA-seq). A set of tools (nucleotide primers, antibodies, microarrays, etc.) for assaying biomarker levels may be packaged into a kit. Results could be used by physicians to make a diagnosis/prognosis and determine the appropriate treatment. Other potential applications of the invention include drug screening and evaluating the efficacy of therapies that aim to inhibit tumor invasion/metastasis.

REST downregulation in weakly invasive MCF-7 breast cancer cells converts them to a more invasive phenotype, while REST overexpression in highly invasive MDA-MB-231 cells suppresses invasiveness. Surprisingly, the mechanism responsible for these phenotypic changes does not depend directly on the transcriptional function of REST protein. Instead, it is driven by previously unstudied mid-size non-coding RNAs (mncRNAs) derived from the first exon of an alternatively REST spliced transcript: REST-003. Processing of REST-003 into mncRNAs is controlled by an uncharacterized serine/arginine repeat-related protein, SRRM3. SRRM3 expression may be under REST-mediated transcriptional control, as it increases following REST downregulation. The SRRM3-dependent regulation of REST-003 processing into mncRNAs has many similarities to recently described promoter-associated small RNA-like processes^{3,4}. Targeting mncRNAs that control invasiveness could lead to new therapeutic approaches to limit breast cancer metastasis.

It is demonstrated that REST downregulation in weakly invasive MCF-7 breast cancer cells converts them to a more invasive phenotype, while REST overexpression in highly invasive MDA-MB-231 cells suppresses invasiveness. The mechanism responsible for these phenotypic changes does not depend directly on the transcriptional function of REST protein. And is driven by mid-size non-coding RNAs (mncRNAs) derived from the first exon of an alternatively REST spliced transcript: REST-003. Data demonstrating that processing of REST-003 into mncRNAs is controlled by an uncharacterized serine/arginine repeat-related protein, SRRM3 is provided. SRRM3 expression may be under REST-mediated transcriptional control, as it increases following REST downregulation. Targeting mncRNAs that control invasiveness could lead to new therapeutic approaches to limit breast cancer metastasis.

### Kits

A kit may comprise instructions for use and one or more markers. Each of the one or more markers may independently comprise at least 70% sequence homology to or at least 70% of the nucleobases or combination thereof of: AGTGTCGGGGCGACTCCCG, GTCGATGTTGGGCCAAATTACCCAATAGC, GTAAATGTGTGCAGTGAGCGGGC, CATTCGGCCATTTTCTCAAAATAC, ATACCAAACACAAAGCAGCTCTTTG, GGCGACTCCCGCGAGTTGGTGTG, GGCATTCCTAACTGAAATAGG, or any fragment thereof, or any combination thereof. The kit may further comprise a database.

### Example

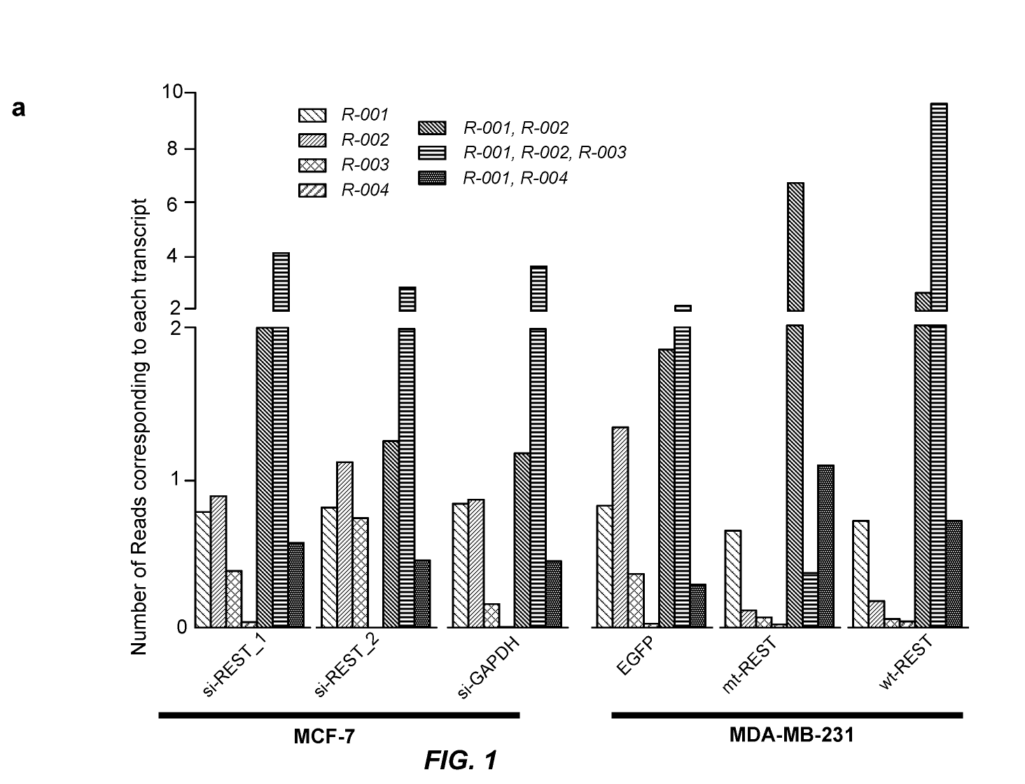

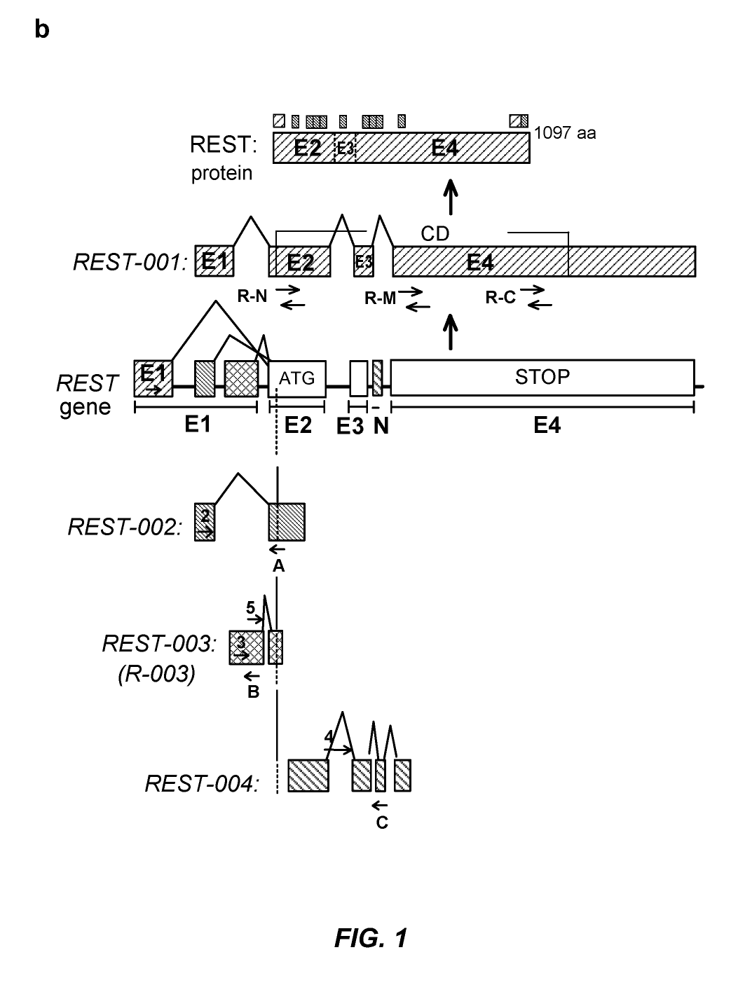

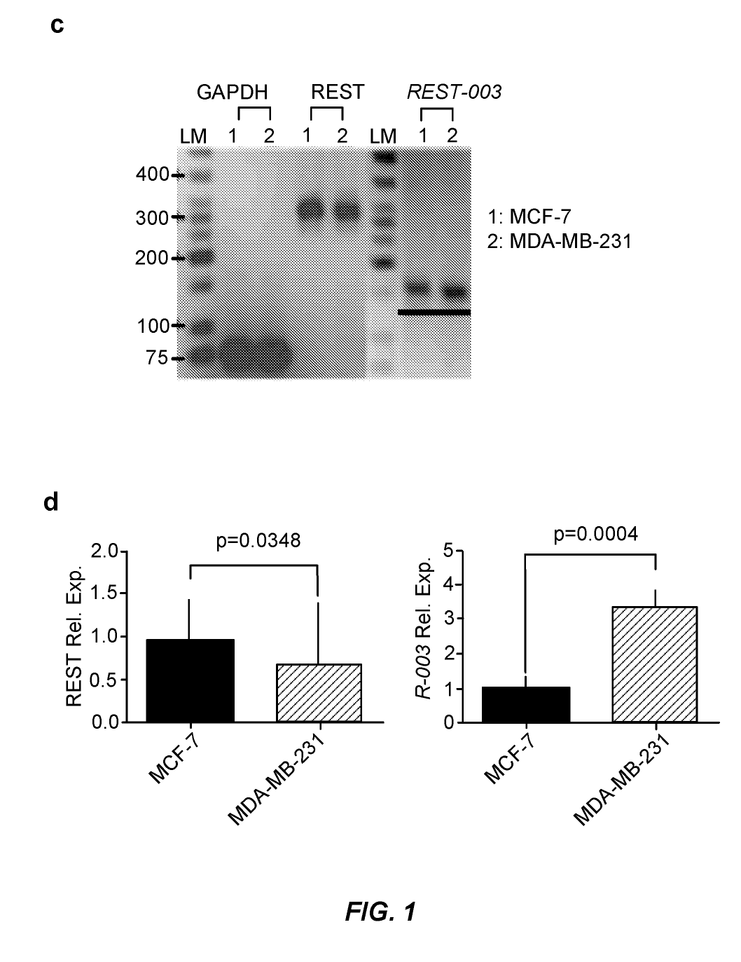

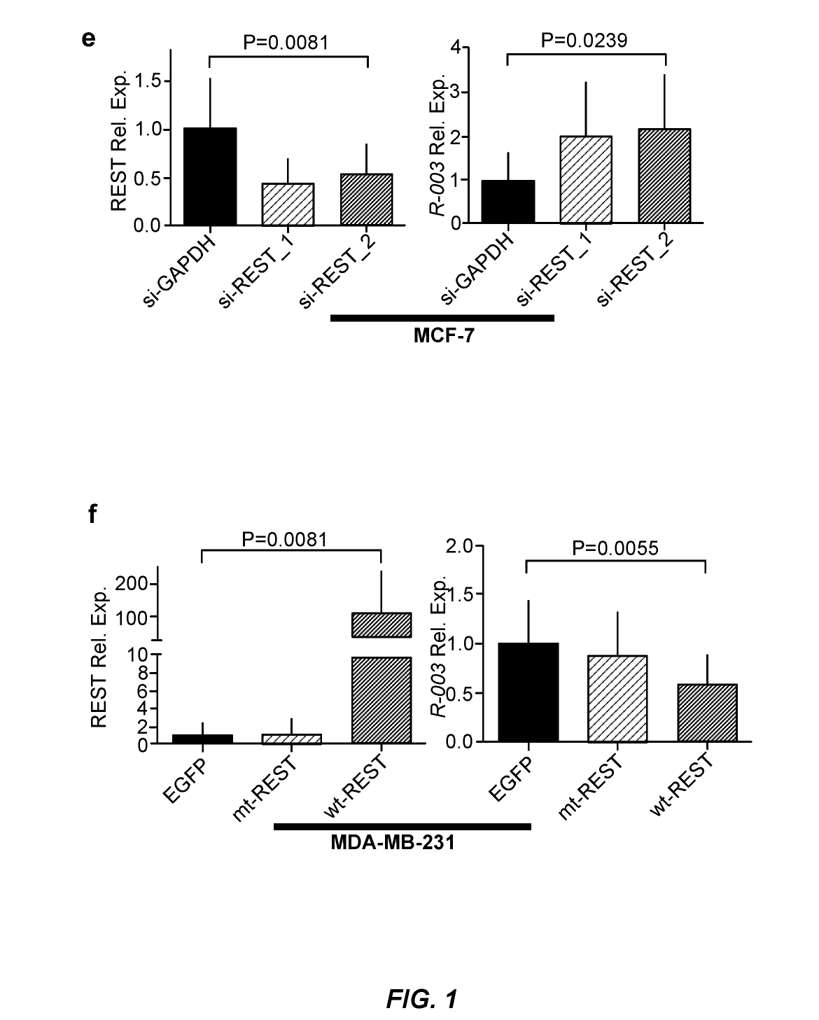

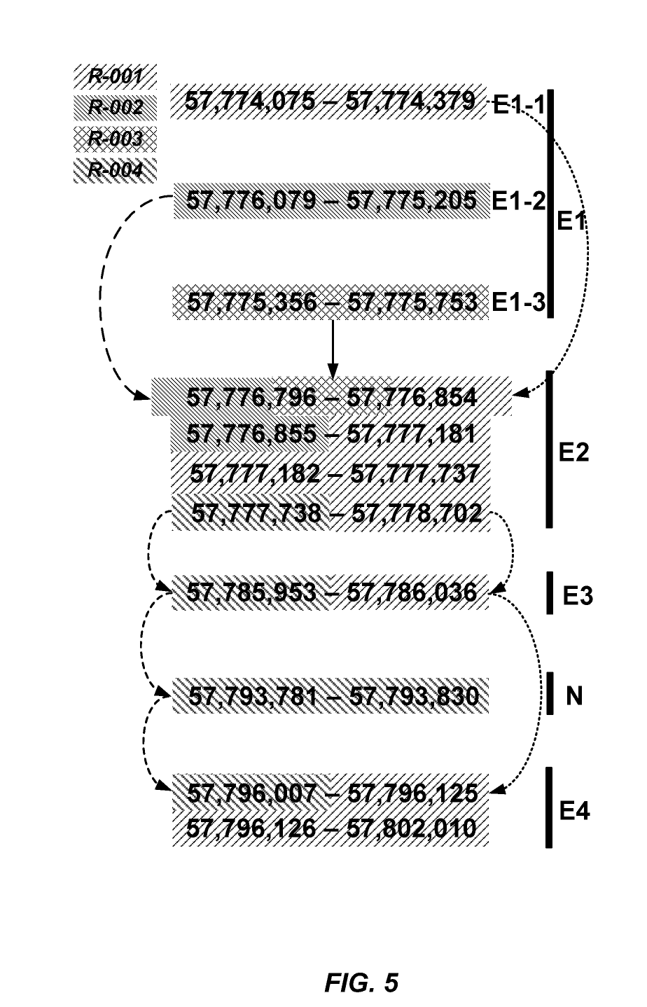

FIG. 1 shows altering REST-003 mncRNA expression in si-REST-treated MCF-7 and REST-overexpressed MDA-MB-231 cells. FIG. 1a shows expression of REST splice variant transcripts (see FIG. 5) in si-RNA-treated MCF-7 and REST-overexpressed MDA-MB-231 cells determined by the pipeline^{14,15} assay of RNA-seq data. FIG. 1b shows schematic diagram of the REST gene and its splice variant transcripts, including illustrations of annotated REST exons and locations of primers employed for the identification of REST splice variants. The constitutive transcript (REST-001; E1-1, E2, E3, and E4) and alternative spliced variants are shown in red, REST-002 in green, REST-003 in yellow, and REST-004 in blue. REST-001 and/or REST-002 can produce the REST protein (wt-REST) that has the complete coding region (E2-4; 1097 aa) containing 9 zinc fingers with DNA binding activity (purple boxes) and two repressor domains (green boxes) necessary for recognizing the RE1 elements and exhibiting repressor function. Forward and reverse primers are indicated by right (numbers) and left (letters) arrows, respectively. FIG. 1c shows detection and FIG. 1d shows expression levels of ncRNA REST-003 (3-B primer pair) and coding REST RNA (R-N or R-M primer pair) in MCF-7 and MDA-MB-231 cells by qRT-PCR. Following PCR amplification, samples were loaded on 4% agarose gel with a 100-bp marker (see FIG. 1c); 1: MCF-7, 2: MDA-MB-231. Expression levels were normalized to GAPDH, CyclophilinA, and/or Actin, converted to MNE, and presented as Relative Expression (Rel. Exp.) after normalization to control samples (MCF-7; n=6 and n=8 biological replicates for REST and REST-003, respectively; n=2 technical replicates per point). Biological replicates are shown on the bar graph as mean plus SEM (paired t-test). FIG. 1e shows the effect of REST on expression of REST-003 ncRNAs in si-REST-treated MCF-7 and FIG. 1f shows the REST-overexpressed MDA-MB-231 cells by qRT-PCR. MCF-7 cells treated with a non-REST si-RNA (si-GAPDH) (see FIG. 1e), and MDA-MB-231 cells transfected with EGFP or mt-REST cDNA²⁹ (lacking two repressor domains) (see FIG. 1f) served as controls. Expression levels were normalized to GAPDH, CyclophilinA and/or Actin, converted to MNE, and presented as Rel. Exp. after normalization to control cells (n=6 biological replicates for REST and REST-003, except n=5 for REST-003 in MCF-7; n=2 technical replicates per point). Biological replicates are shown on the bar graph as mean plus SEM (one-way ANOVA with Friedman test for multiple comparisons).

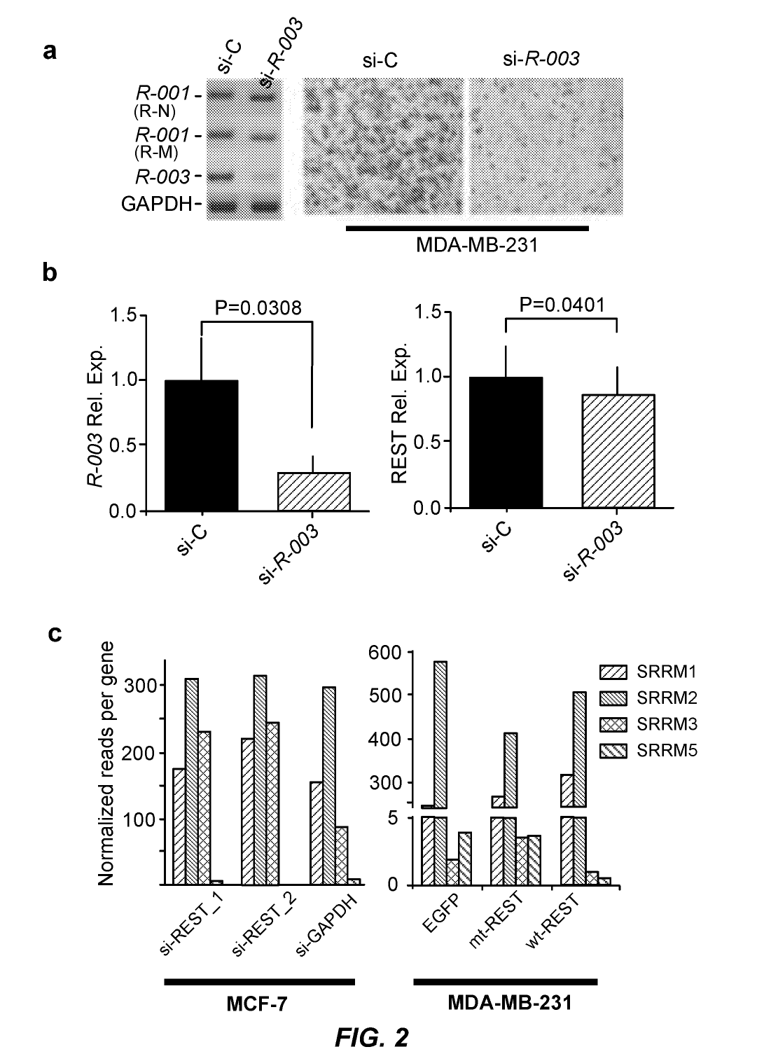

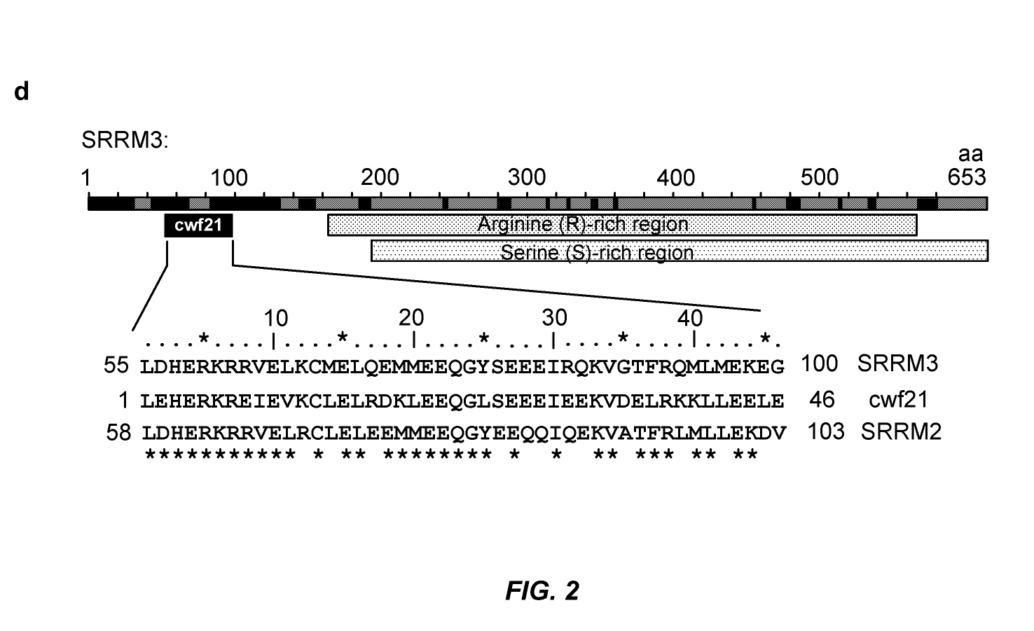

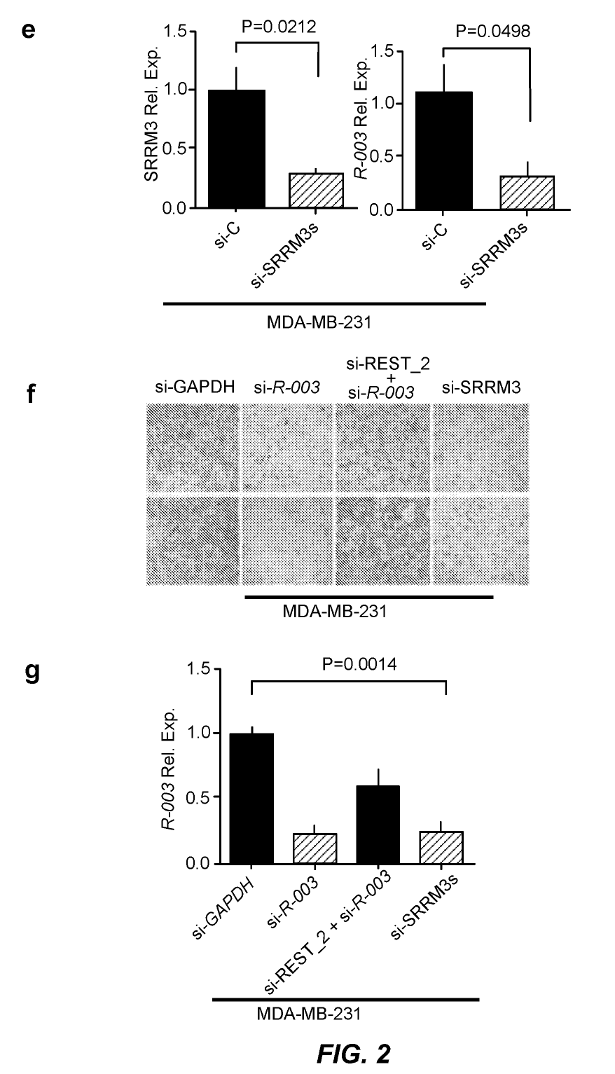

FIG. 2 shows the effect of si-REST-003 on MDA-MB-231 invasiveness and SRRM3 expression data from RNA-seq experiments on si-REST-treated MCF-7 and REST-overexpressed MDA-MB-231 cells. FIG. 2a shows detection of REST-003 ncRNA by qRT-PCR (left) and invasiveness by a Matrigel invasion chamber (right) after treating MDA-MB-231 cells with si-REST-003. FIG. 2b shows reduced REST-003 expression by si-REST-003 treatment in MDA-MB-231 cells using qRT-PCR. More than 50% of REST-003 transcripts were reduced by si-REST-003 relative to si-C (scramble). REST transcript expression (REST-001, R-M primer pair) was not changed by si-REST-003 treatment. Expression levels were normalized to GAPDH, CyclophilinA and/or Actin, converted to MNE, and presented as Rel. Exp. after normalization to si-C samples (MCF-7; n=7 and n=6 biological replicates for REST and REST-003, respectively; n=2 technical replicates per point). Biological replicates are shown on the bar graph as mean plus SEM (paired t-test). FIG. 2c shows expression of different SRRM subfamilies in si-RNA-treated MCF-7 and REST-overexpressed MDA-MB-231 cells by the pipeline analysis^{14,15} of RNA-seq. FIG. 2d shows N-terminal sequences of SRRM3, cfw2l (S. cerevisiae), and SRRM2 (H. sapiens) were compared using the ClustalW2 program. Identical residues in cwf2l domains are in red font for all three SR-related proteins. Stars indicate identical residues in SRRM3 and SRRM2. FIG. 2e shows the effect of si-SRRM3 on REST-003 ncRNA expression in MDA-MB-231 cells using qRT-PCR. Expression levels were normalized to GAPDH, CyclophilinA and/or Actin, converted to MNE, and presented Rel. Exp. after normalization to si-C samples (MCF-7; n=7 biological replicates for REST and REST-003; n=2 technical replicates per point). Biological replicates are shown on the bar graph as mean plus SEM (paired t-test). FIG. 2f shows the effect of different siRNA treatments on MDA-MB-231 Matrigel invasiveness and FIG. 2g shows REST-003 ncRNA expression. Expression levels were normalized to GAPDH, CyclophilinA and/or Actin, converted to MNE, and presented as Rel. Exp. after normalization to control cells (n=3 biological replicates for REST-003; n=2 technical replicates per point). Biological replicates are shown on the bar graph as mean plus SEM (one-way ANOVA with Dunnet test for multiple comparisons).

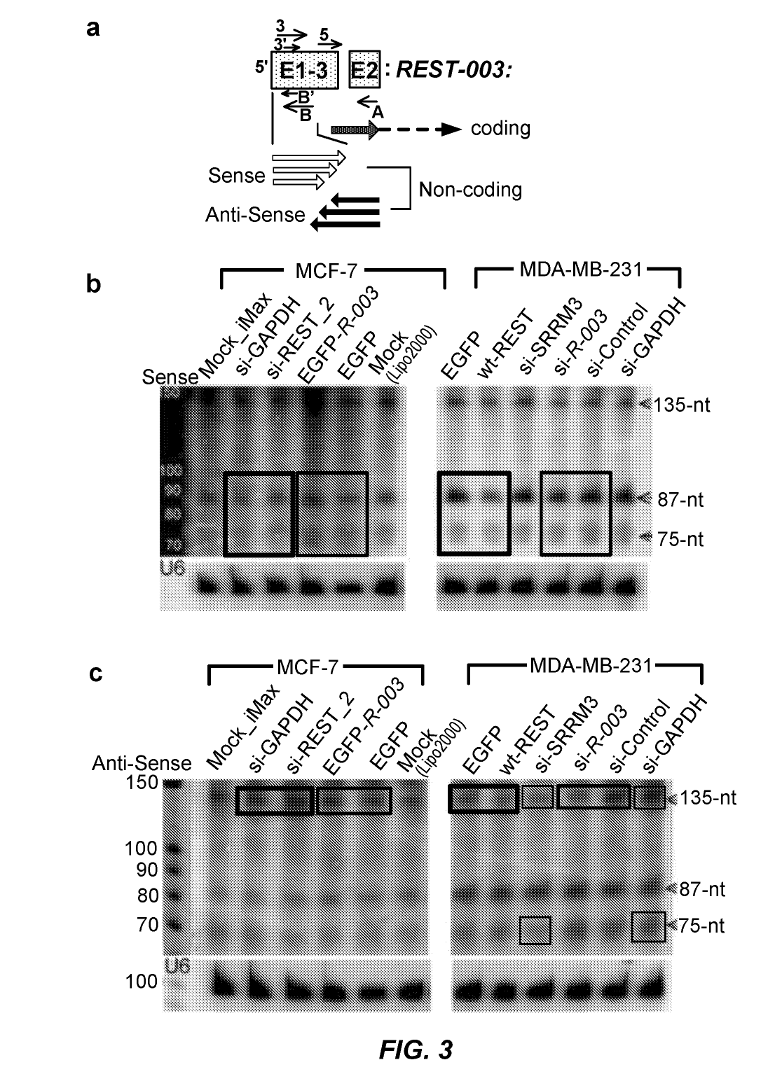

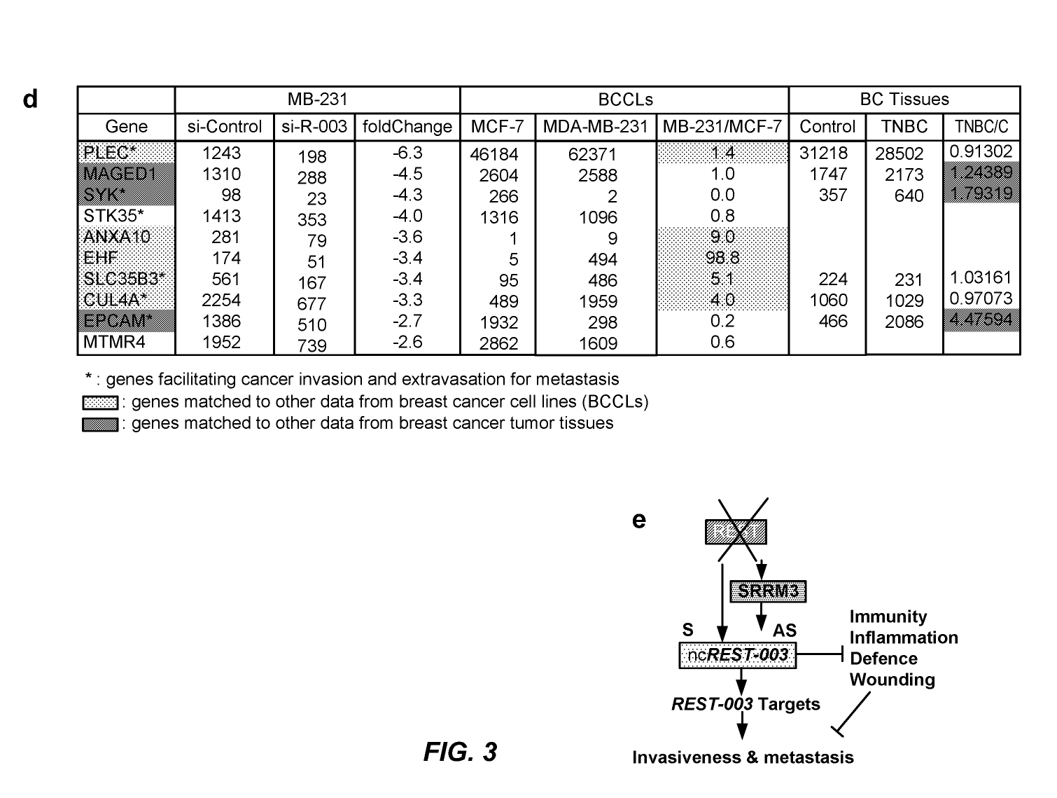

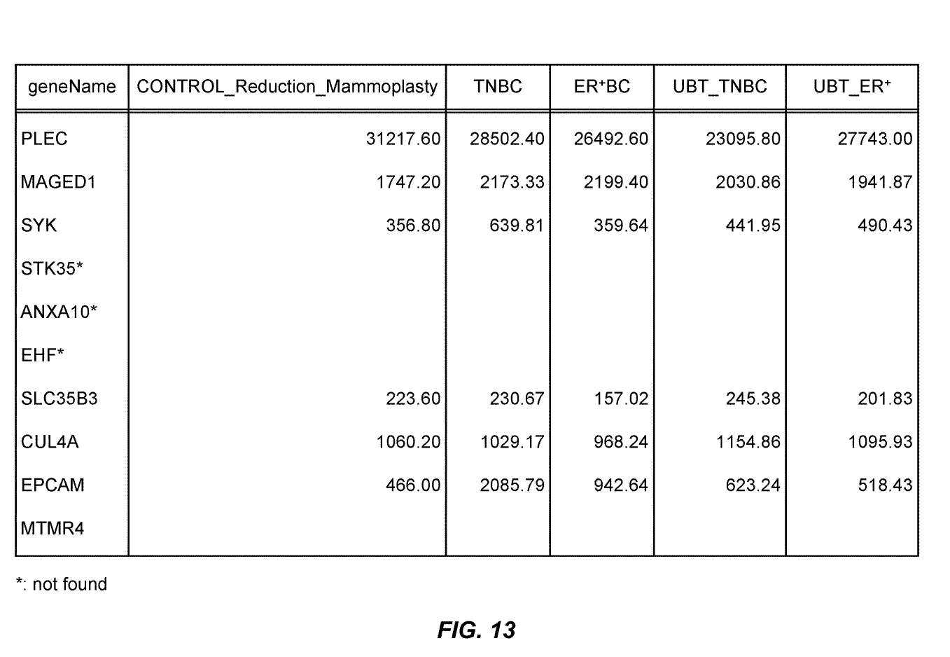

FIG. 3 shows the expression pattern of REST-003 and its downregulating effect on MDA-MB-231 cells. FIG. 3a shows schematic picture of ncRNAs and coding RNAs transcribed from the E1-3 region. Many new sncRNAs that are enriched at the 5′ boundary of the REST gene (E1-3) are predicted as S (yellow) and AS (purple) sequences. FIGS. 3b and 3c show differential expression of REST-003 in MCF-7 and MDA-MB-231 cells using northern blot analysis. RNA samples were prepared from each cell line transfected with controls and different siRNAs, as indicated. Hybridizations were performed using ³²P-labeled DNA oligonucleotide probes complementary to the S and AS transcripts probes: AS (B*) sequence of E1-3 for S (see FIG. 3b) and S (3*) sequence for AS (see FIG. 3c) detection (Supplementary Information). Human U6 RNA (˜105 nt) was probed as an internal control. FIG. 3d shows genes downregulated by REST-003 downregulation. Effect of REST-003 ncRNA downregulation on expression of REST target genes and/or other genes related to invasiveness using RNA-seq analysis. The level of gene expression in si-REST-003-treated MDA-MB-231 cells was compared to that of control (si-C) cells. Downregulated gene expression in si-REST-003-treated cells is shown using DESeq from the pipeline (adjusted P-value<0.05; yellow box). The downregulated genes were compared with published RNA-seq data from MCF-7 and MDA-MB-231 cell lines (BCCLs, purple box), 42 triple negative (TNBC) tissues, and 58 nonmalignant control tissues (blue box)²⁵. For tissue data, expression reads of downregulated genes by si-REST-003 treatment were averaged from TNBC and controls by the pipeline^{14,15} assay of RNA-seq data²⁵ (FIG. 13) and compared to each other. FIG. 3e shows a schematic of the regulatory interactions among REST, REST-003, and SRRM3 that may coordinate gene regulation required for development of the invasive phenotype.

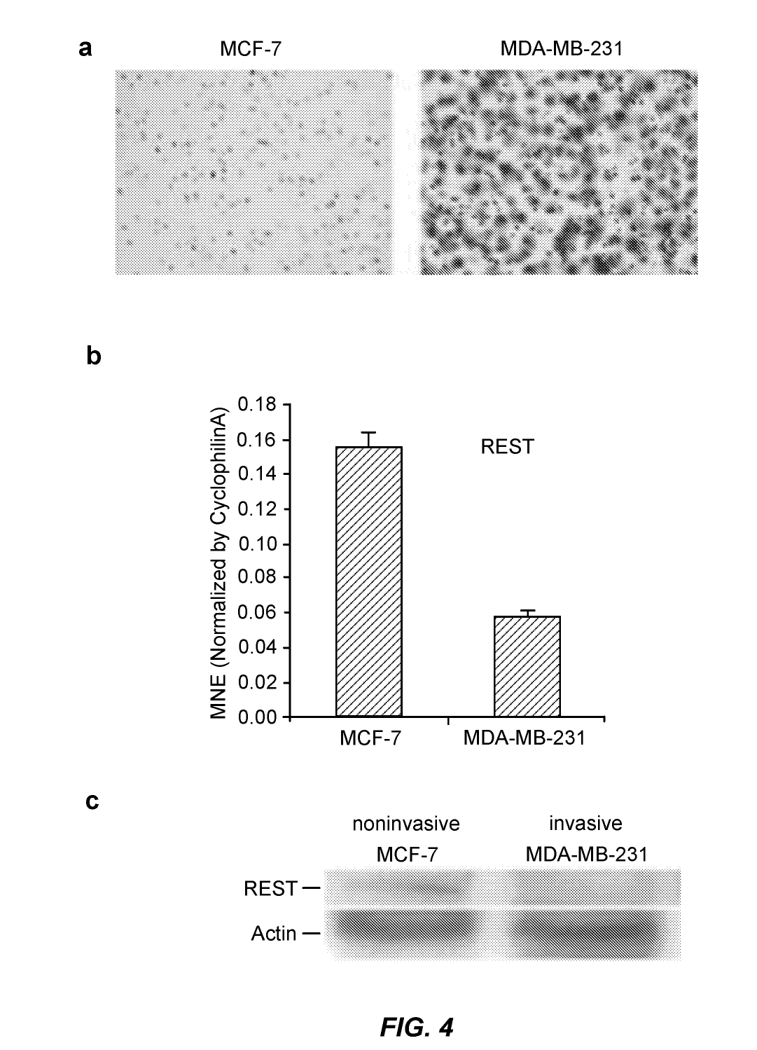

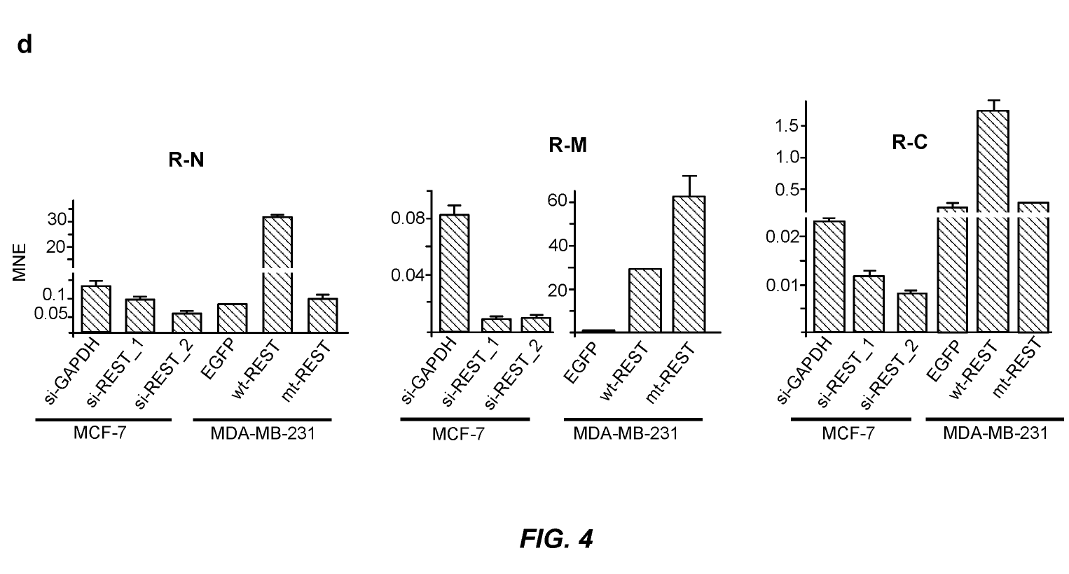

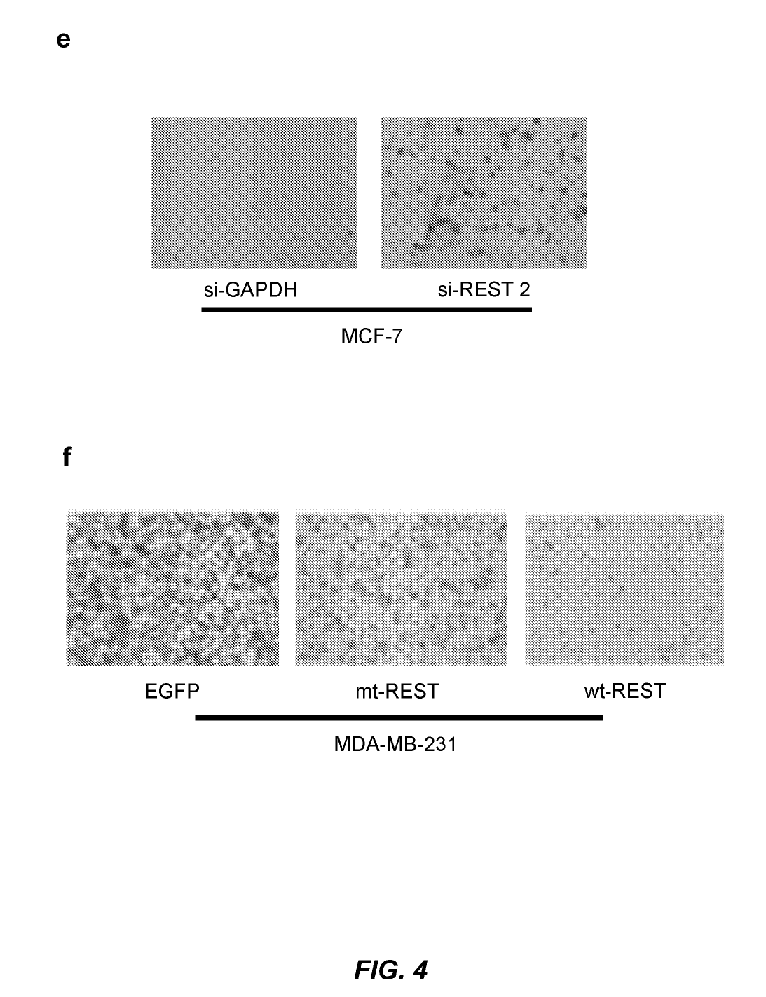

FIG. 4 shows the differences in REST expression and invasiveness between MCF-7 and MDA-MB-231 cells. FIG. 4a shows invasive potential of each cell line determined by a Matrigel Invasion Chamber assay. MCF-7 cells are normally not invasive, while MDA-MB-231 cells are highly invasive. Purple cells in the images have invaded and moved across the Matrigel barrier. FIG. 4b shows expression of REST transcript and FIG. 4c shows REST protein by qRT-PCR and western blot, respectively, in MCF-7 and MDA-MB-231 cells. FIG. 4d shows alteration of REST mRNA expression by siRNA and cDNA treatment in both cell lines using qRT-PCR. Approximately 60-80% of REST expression was reduced by siRNAs against REST (si-REST_1 or si-REST_2) in MCF-7 cells. MDA-MB-231 cells transfected with wild-type (wt) REST cDNA showed higher REST mRNA expression relative to control cells transfected with EGFP cDNA. REST-N(R-N) and REST-C(R-C) primers can distinguish expression of wt- and mt-REST expression in MDA-MB-231 cells compared with REST-M (R-M) primers. Their expression levels were normalized to the housekeeping genes, GAPDH and/or Cyclophillin (MNE). Error bars indicate SEM (n=5 for each experiment). Since si-REST_2 was more effective than si-REST_1, si-REST_2 was chosen in further experiments. FIG. 4e shows effect of REST downregulation on Matrigel invasiveness in MCF-7 cells. These cells became invasive after si-REST_2 treatment. FIG. 4f shows overexpression of wt-REST reduced invasiveness of MDA-MB-231 cells. Overexpression of mt-REST reduced invasiveness relative to a control (EGFP) but not to the same degree as wt-REST. Representative images are shown in all cases.

FIG. 5 shows the bioinformatics at the REST gene locus. Data were retrieved from the UCSC or Ensemble Genome Browser 75 (http://uswest.ensembl.org/index.html). Annotated REST exons and their splicing images are illustrated. The first exon (E1) contains three different parts (E1-1, E1-2 and E1-3) to be spliced out and connected to E2. The constitutive transcript (REST-001; E1-1, E2, E3, and E4) and alternative spliced variants are shown in red and different colors, respectively (green for REST-002, yellow for REST-003, and blue for REST-004): REST-002; E1-2 to E2, REST-003; E1-3 to E2. REST-004 contains truncated E2, E3, Exon N, and truncated E4.

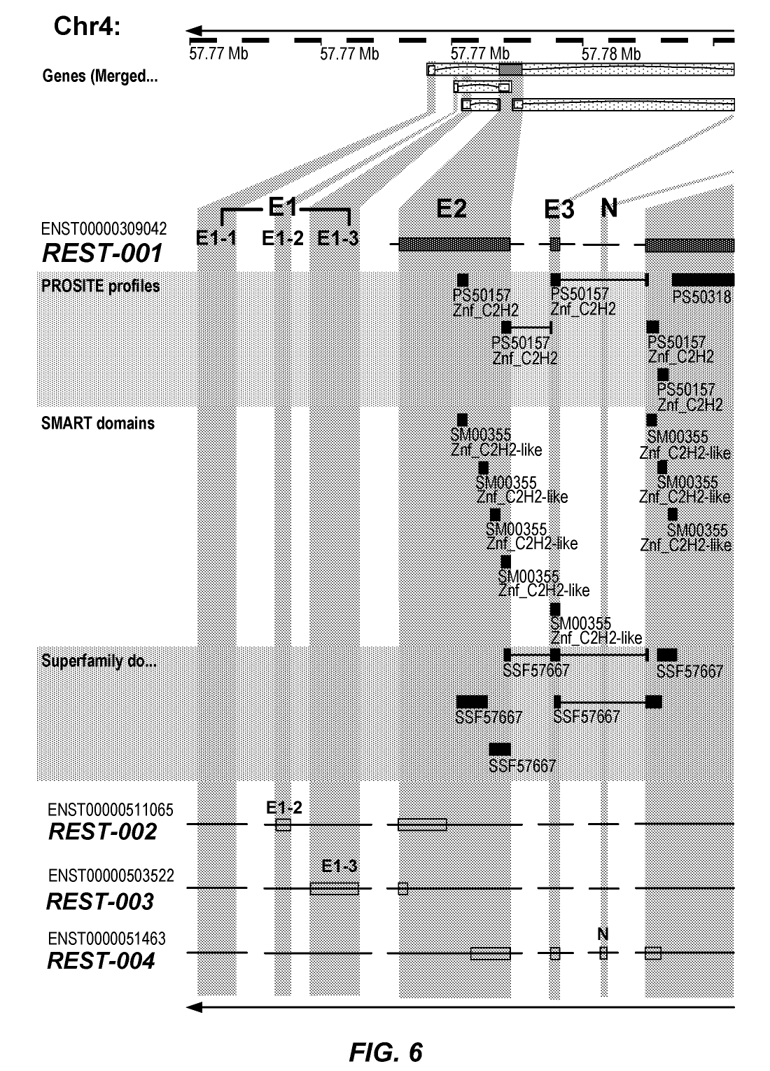

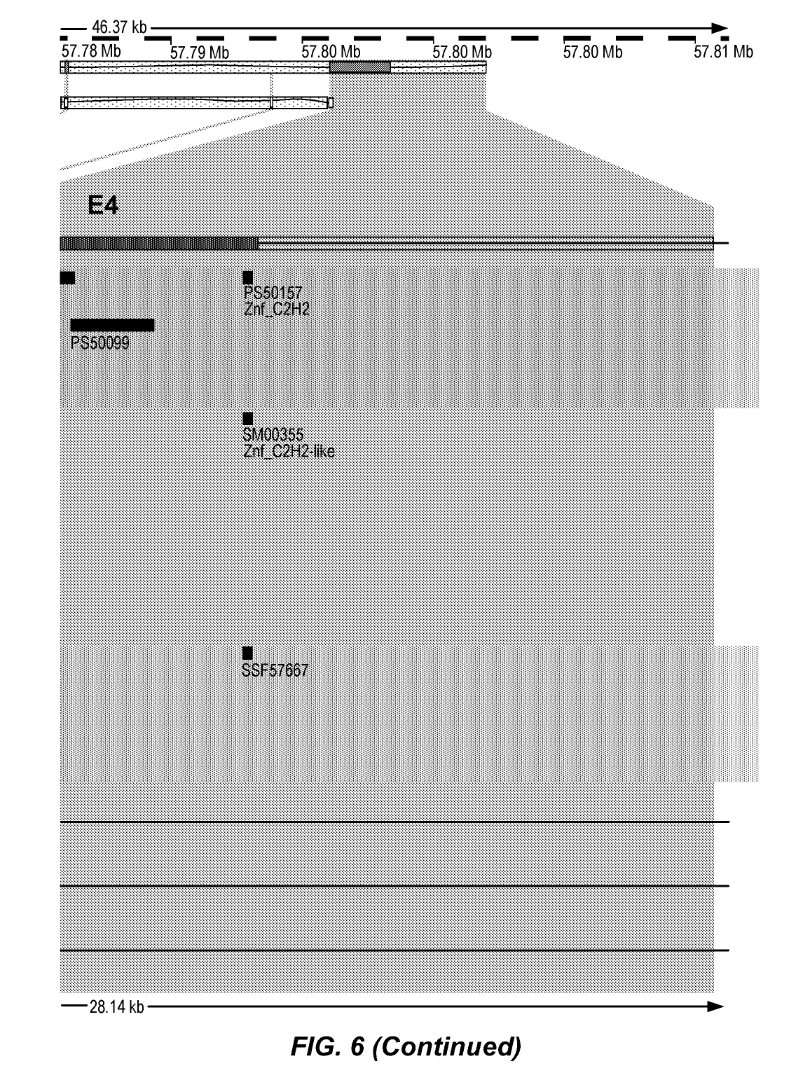

FIG. 6 shows EnsEMBL_Web_Component_Gene_SpliceImage-Homo_sapiens-Gene-Splice-73-ENSG00000084093.

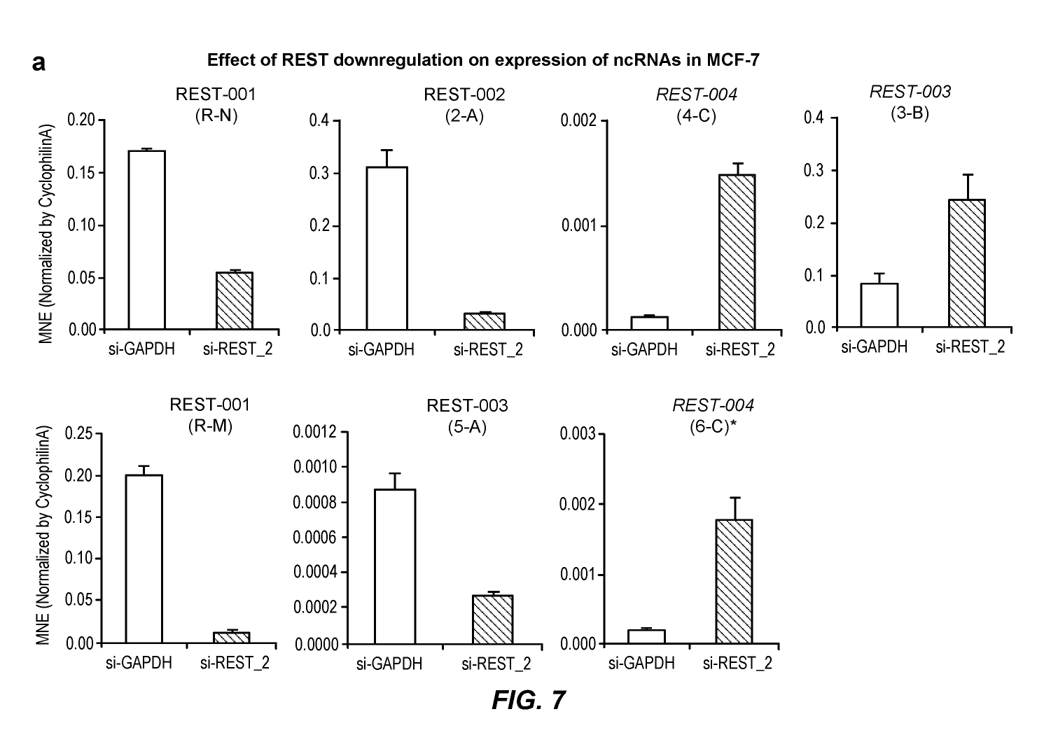

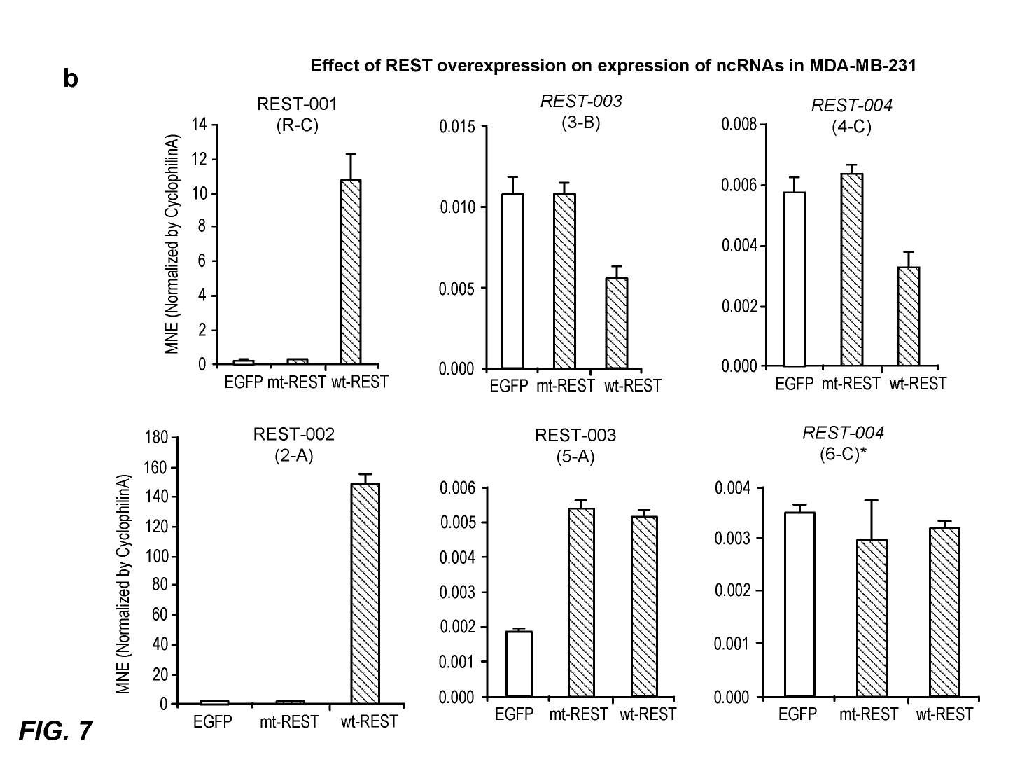

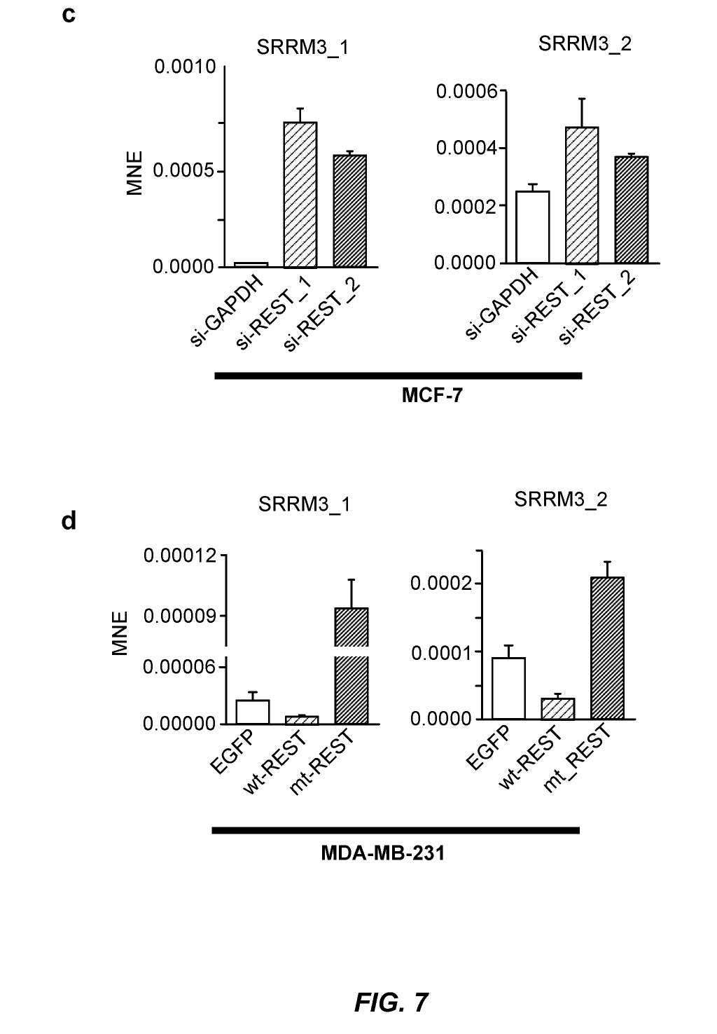

FIG. 7a shows the effect of REST downregulation in MCF-7 and FIG. 7b shows REST overexpression in MDA-MB-231 on expression of ncRNAs by qRT-PCR. Expression of ncRNAs increases following REST downregulation in MCF-7 (see FIG. 7a) and decreases following REST overexpression in MDA-MB-231 (see FIG. 7b) relative to the controls. In contrast, expression of coding RNAs produces the opposite behavior. FIG. 7c shows effect of REST downregulation in MCF-7 (left) and REST overexpression in MDA-MB-231 (right) on expression of SRRM3 by qRT-PCR. Expression levels were normalized to the housekeeping genes, GAPDH and/or Cyclophilin (MNE). Error bars indicate SEM (n=2 for each experiment).

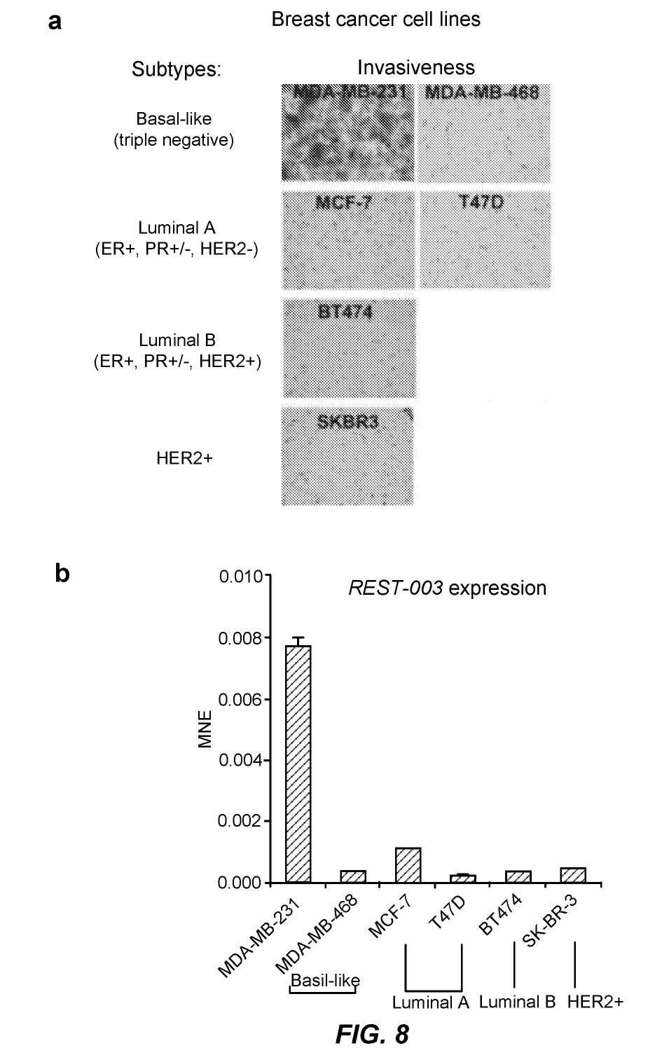

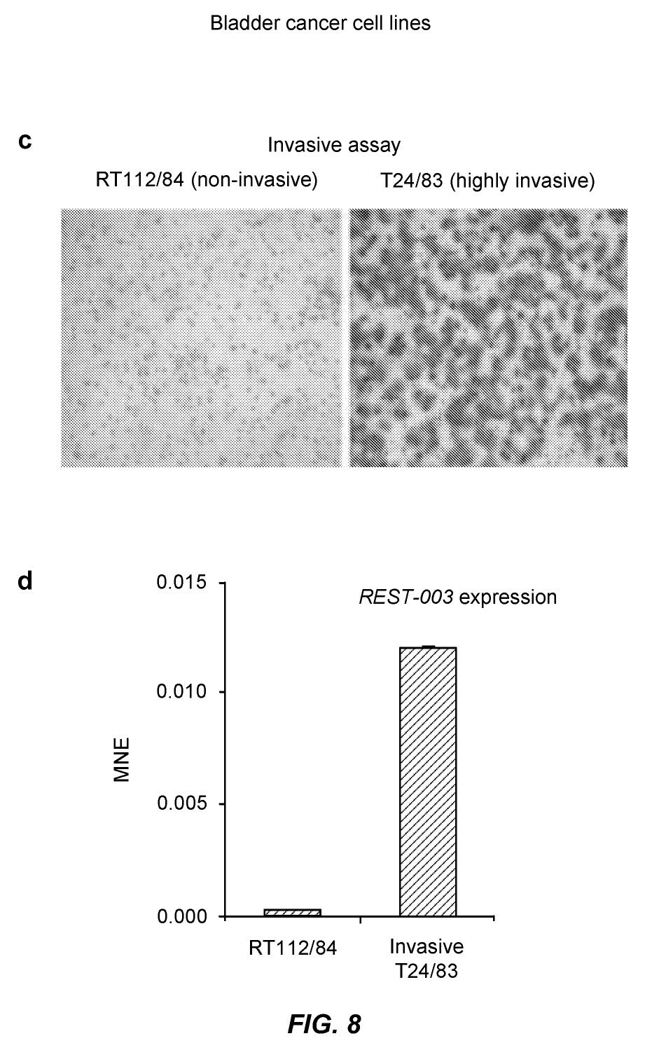

FIG. 8 shows the positive correlation between REST-003 expression and invasiveness in several breast cancer cell lines and bladder cancer cell lines. FIG. 8a shows invasive potential of each cell line was determined by a Matrigel Invasion Chamber assay. Purple cells in the images have invaded and moved across the Matrigel barrier. Cell lines were classified into four subtypes: luminal A, luminal B, HER2+, and basal-like (triple negative). Immunoprofiles of each subtype are provided. FIG. 8b shows expression of REST-003 in each cell line as determined by qRT-PCR. REST-003 was highly expressed in invasive MDA-MB-231 cells but not in other cell lines that exhibit no Matrigel invasion. FIGS. 1c and 1d show positive correlation between invasiveness and REST-003 expression in bladder cancer cell lines. Invasive bladder cancer cells (T24/83) expressed REST-003 at higher levels than noninvasive ones (RT112/84).

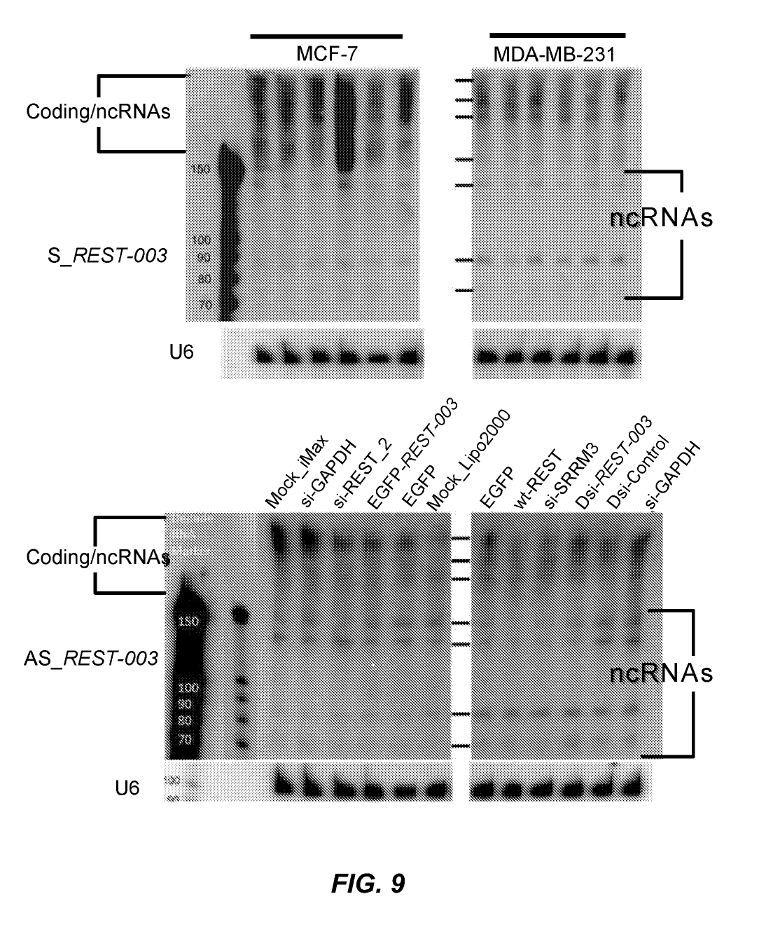

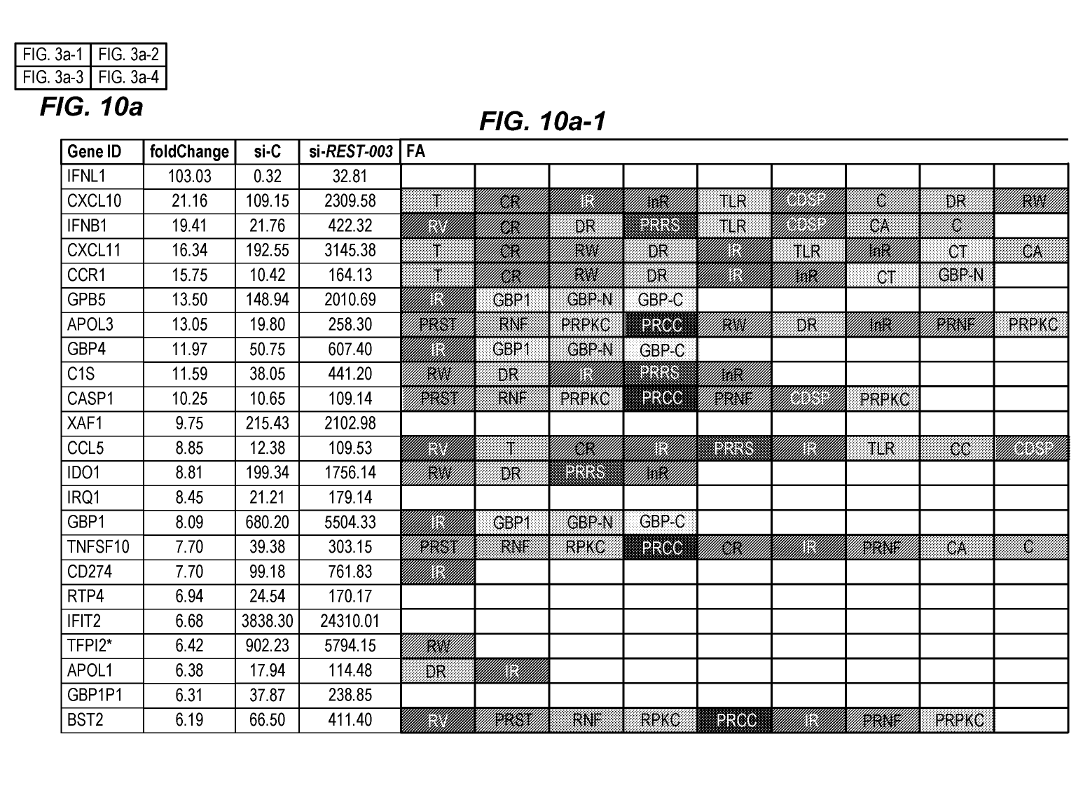

FIG. 9 shows a northern gel picture of differential expression of REST-003 in MCF-7 and MDA-MB-231 cells. At least five larger (>200 nt) REST-003 S and AS bands are highly expressed in MCF-7 cells, similar to REST-001 expression.

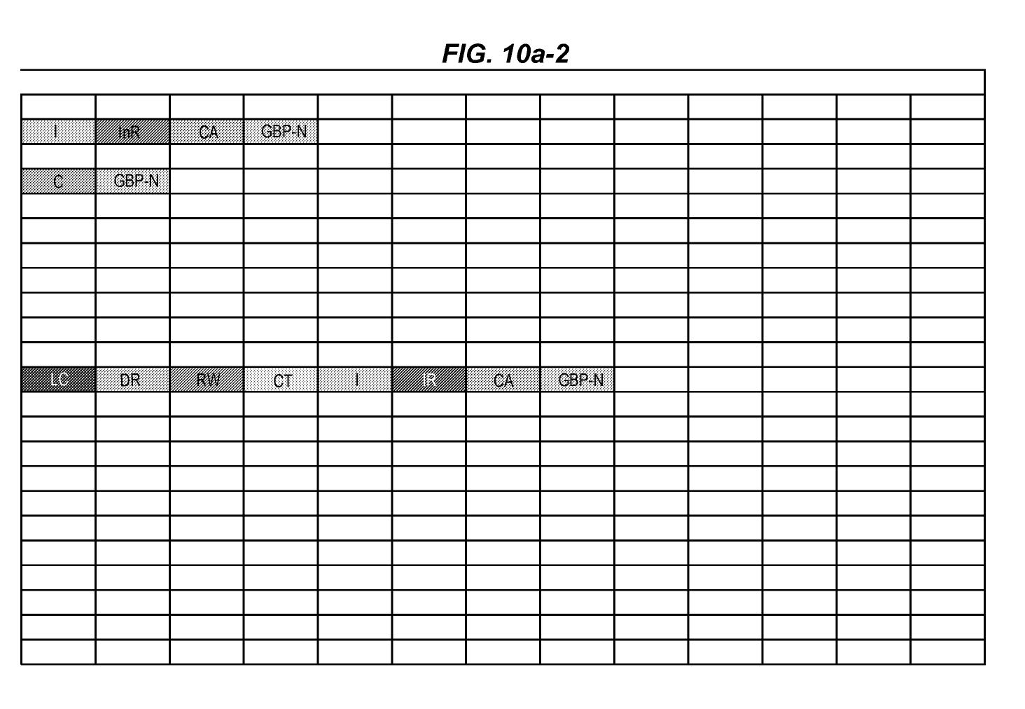

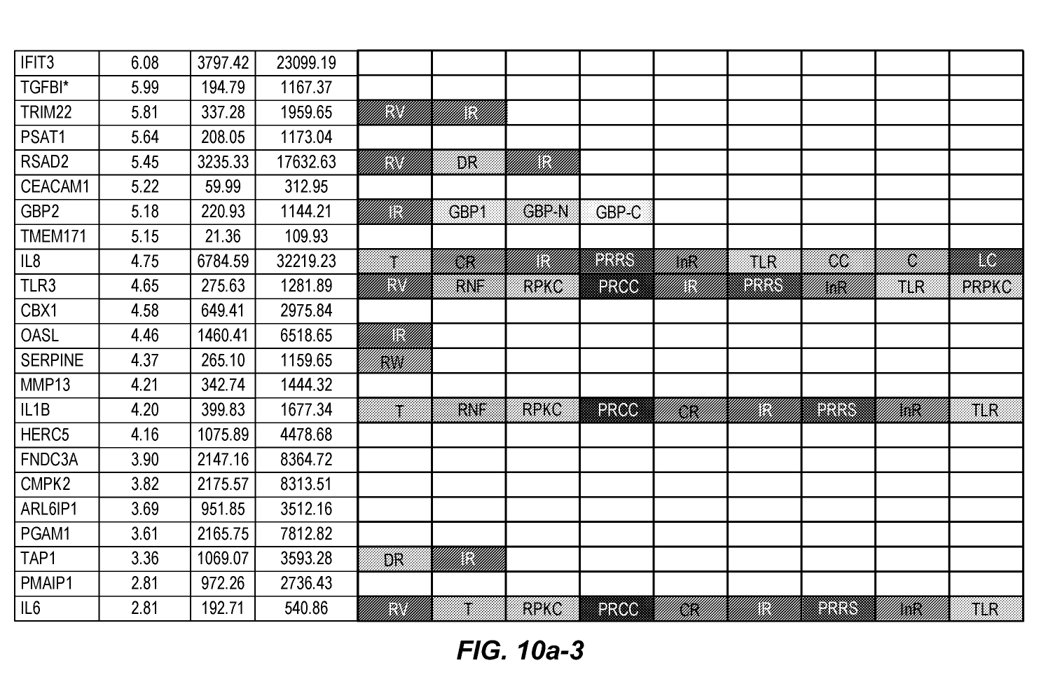

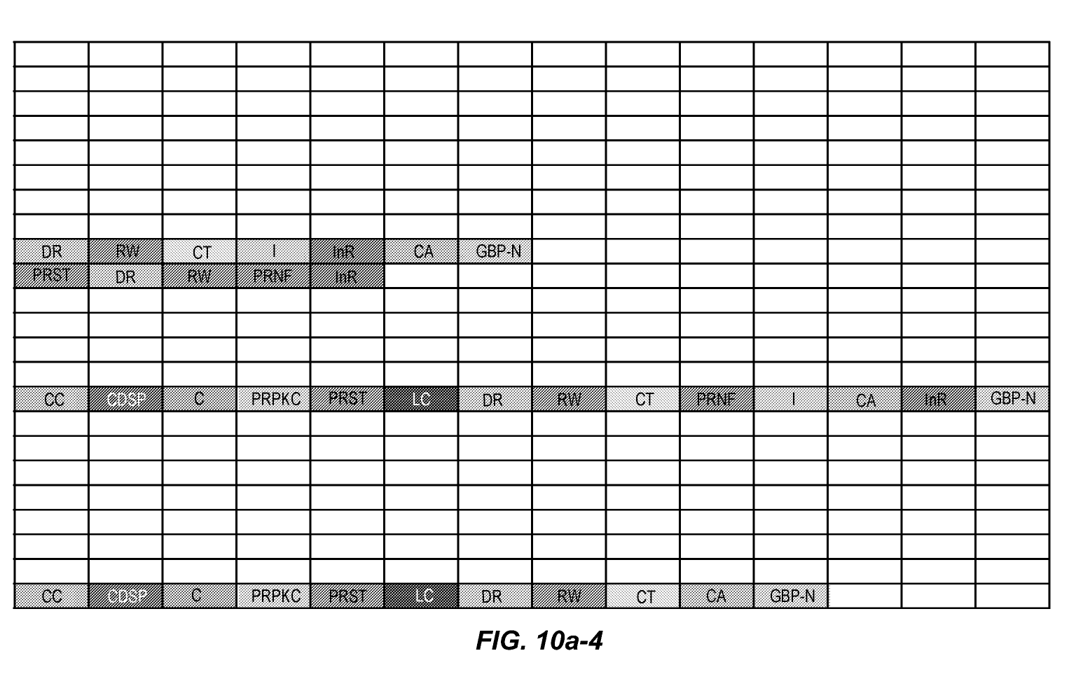

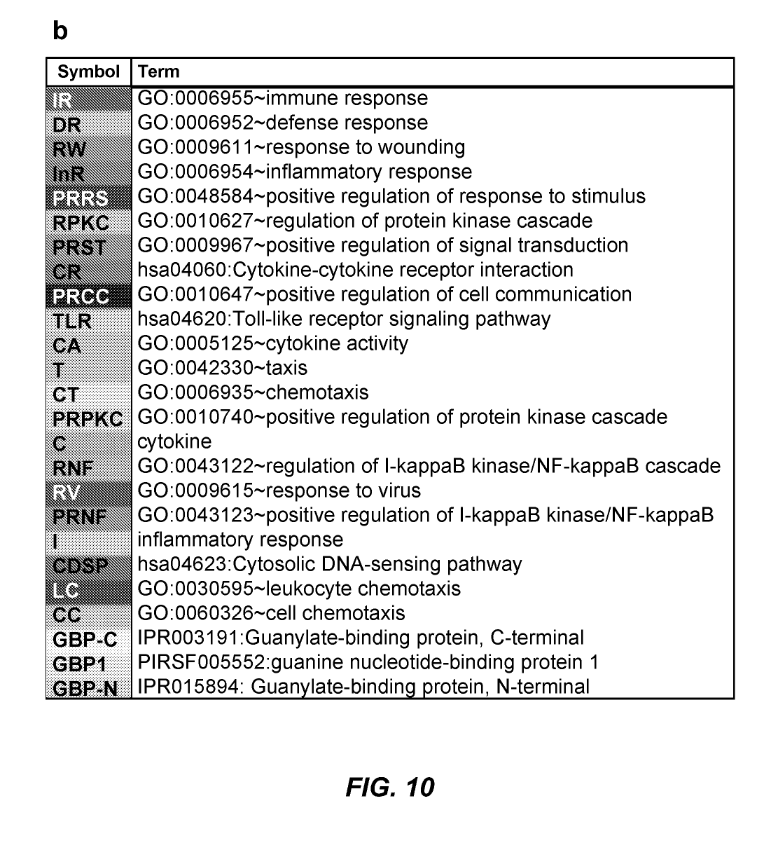

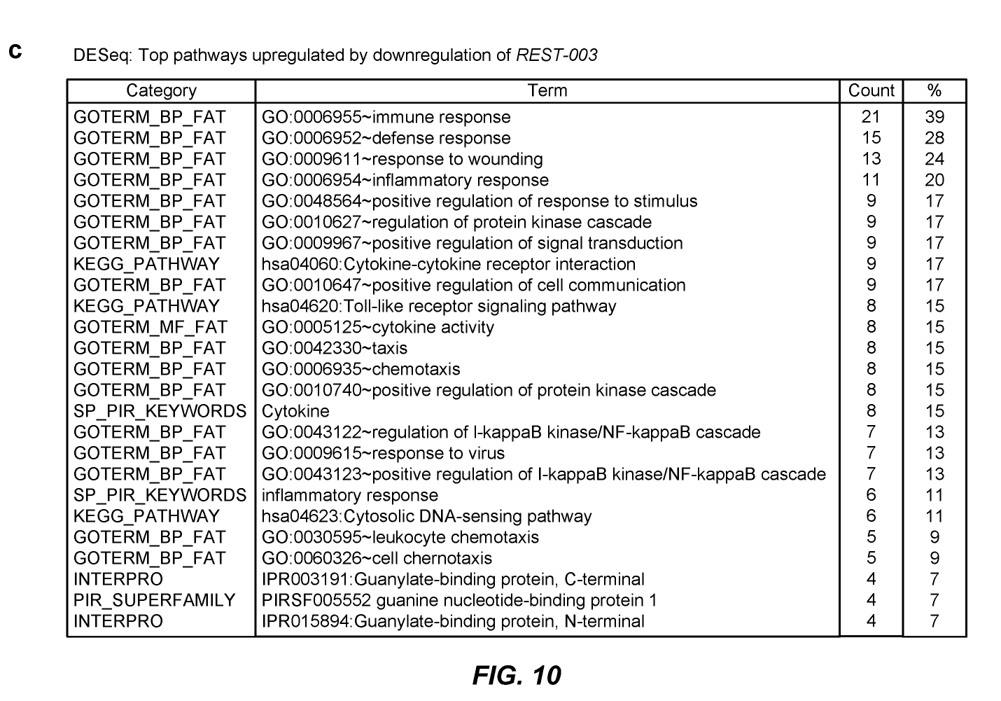

FIG. 10 shows upregulated genes and their pathways following si-REST-003 treatment in MDA-MB-231 cells. The level of gene expression in si-REST-003-treated MDA-MB-231 cells was compared with that of control (si-C) cells. Upregulated gene expression in si-REST-003-treated cells is shown using DESeq from the pipeline (adjusted P-value<0.05) and functional analysis from DAVID (FDR<0.05). Different functions of genes are represented with different colors (see FIG. 10a) and terms (see FIG. 10b). FIG. 10c shows top pathways upregulated by downregulation of REST-003 in MDA-MB-231 cells are shown using DESeq from the pipeline (adjusted P-value<0.05).

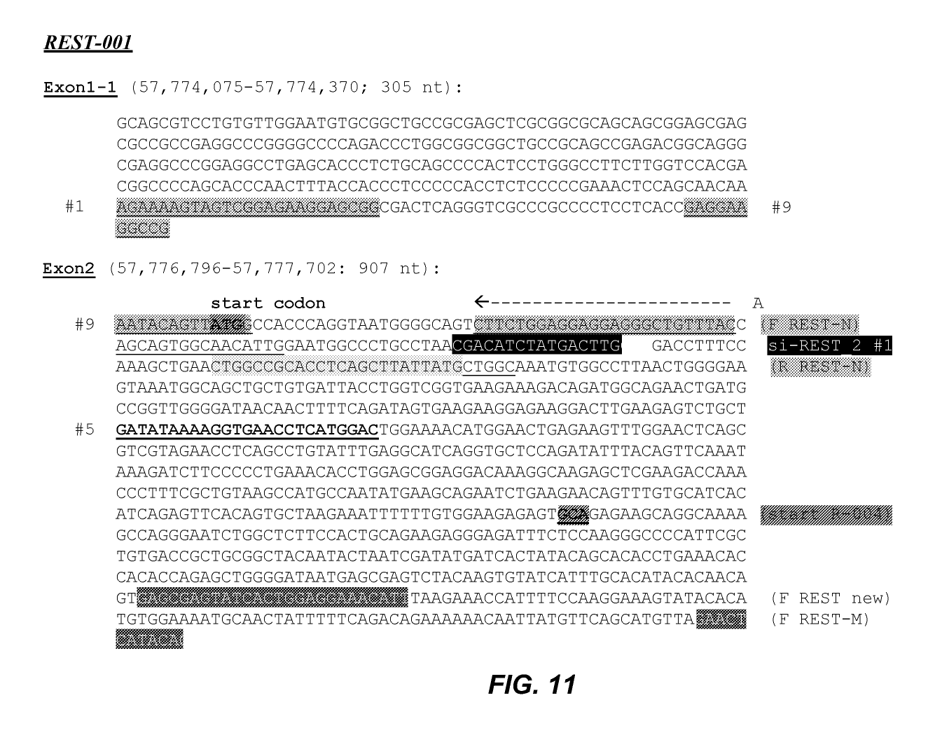

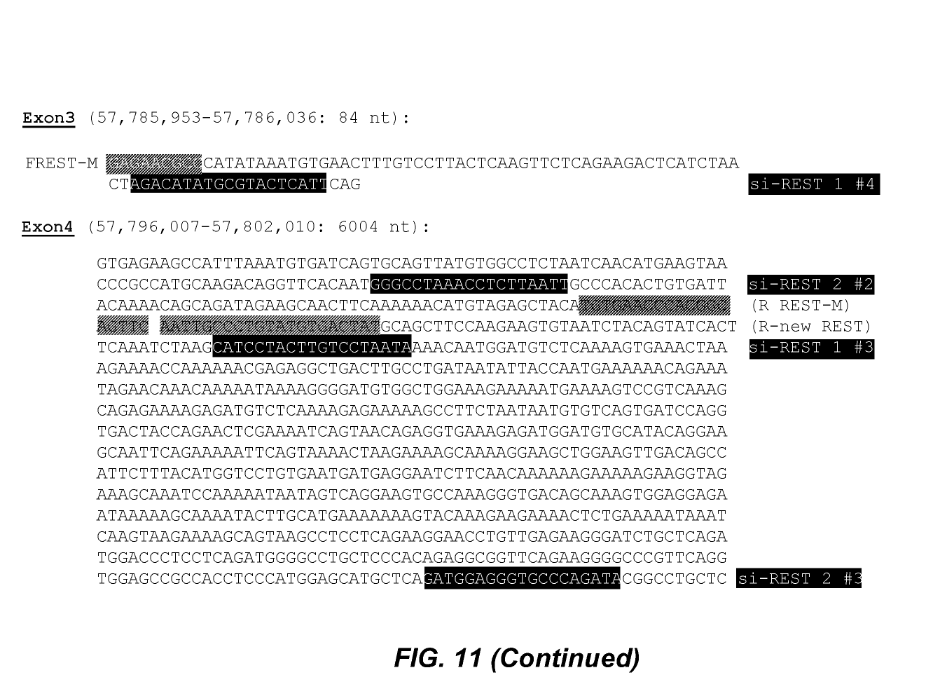

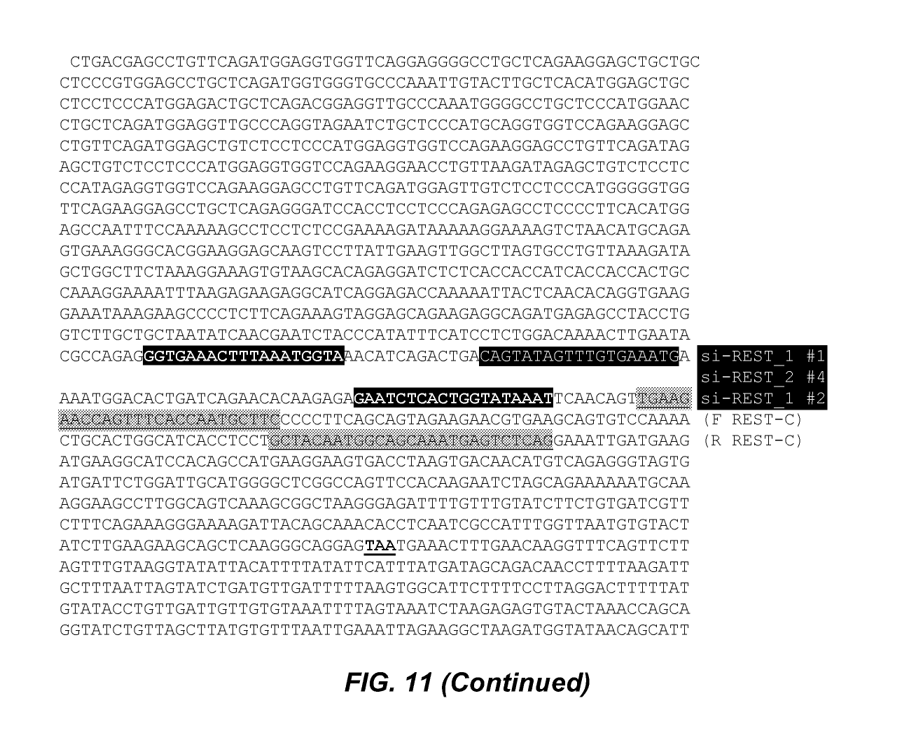

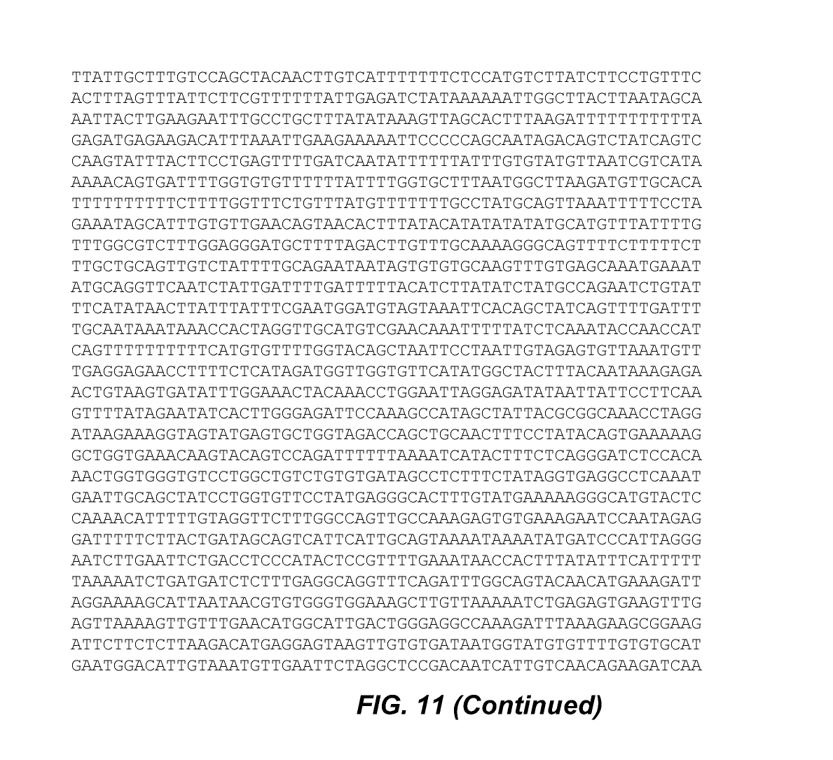

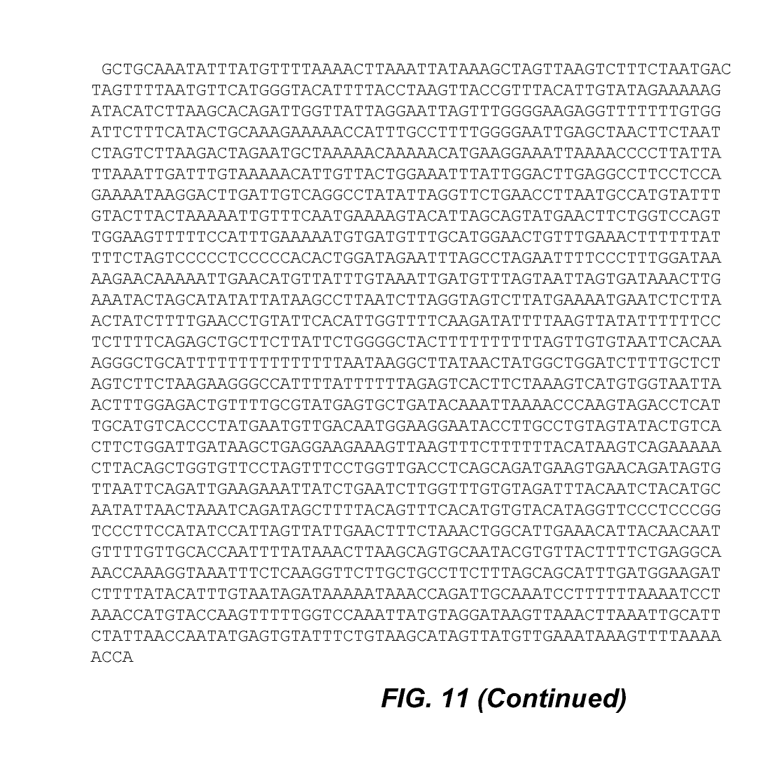

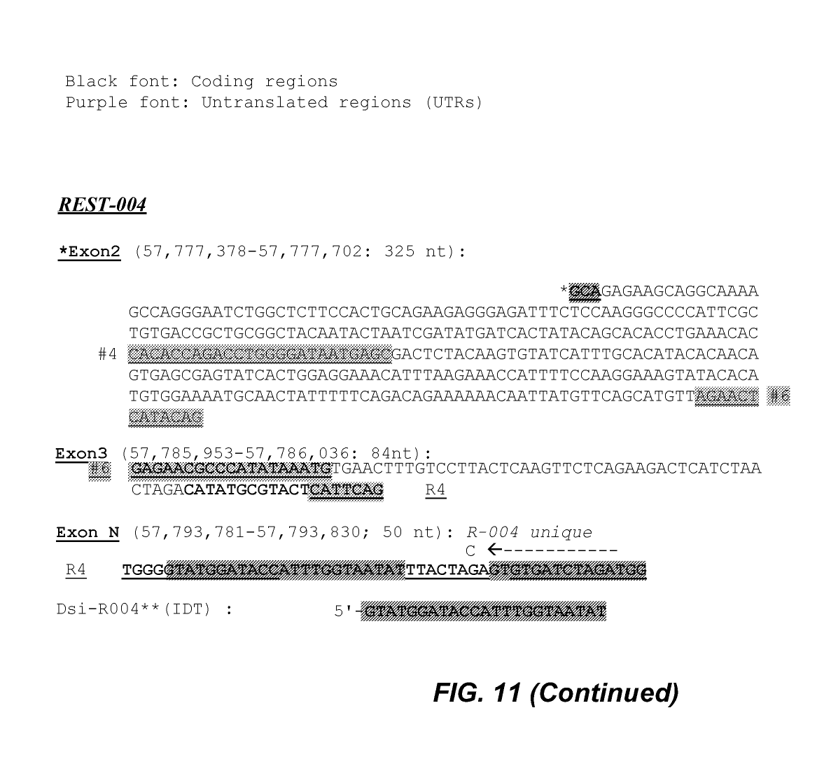

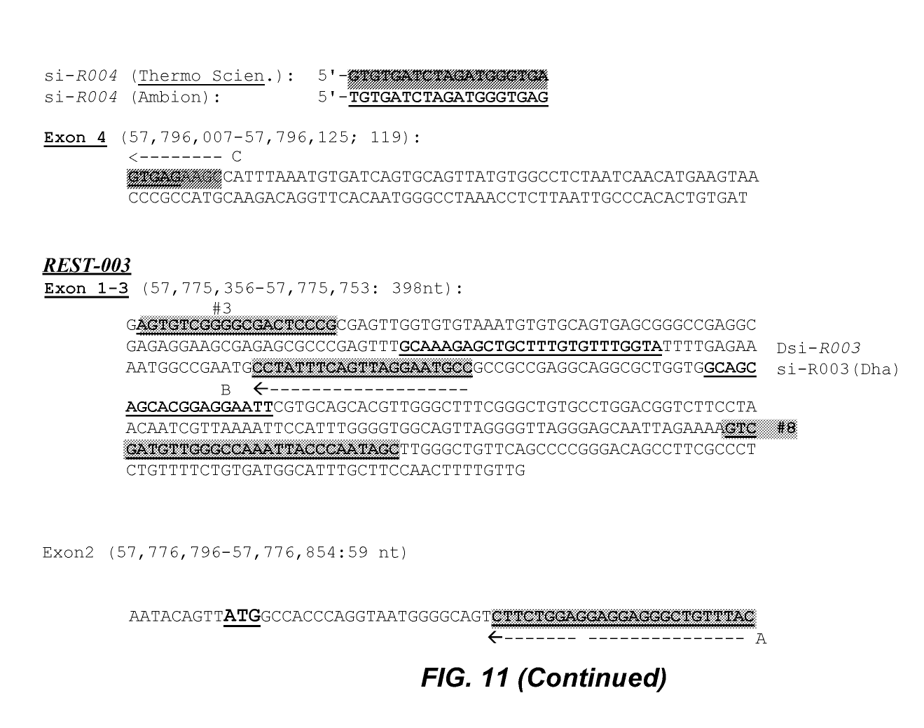

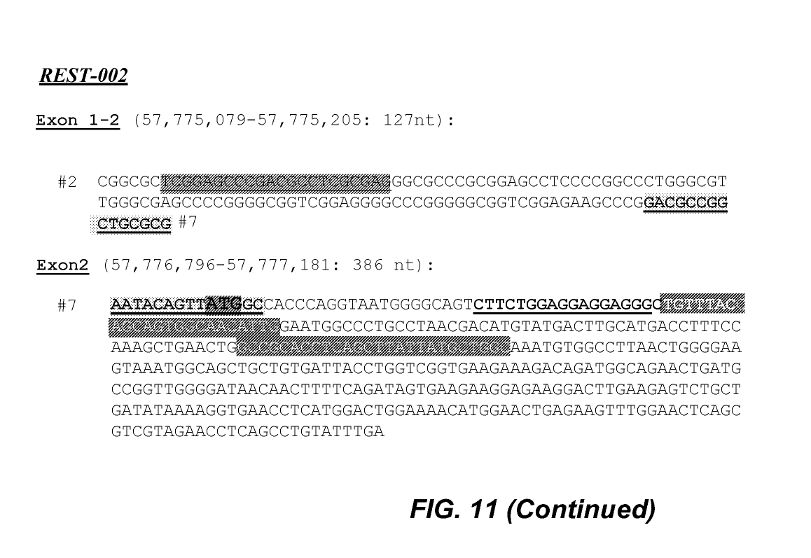

FIG. 11 shows REST-002 transcript variant differs in the 5′ UTR compared to REST-001, but REST-001 and REST-002 variants encode the same REST protein. Therefore, the focus was not on non-coding RNA REST-002.

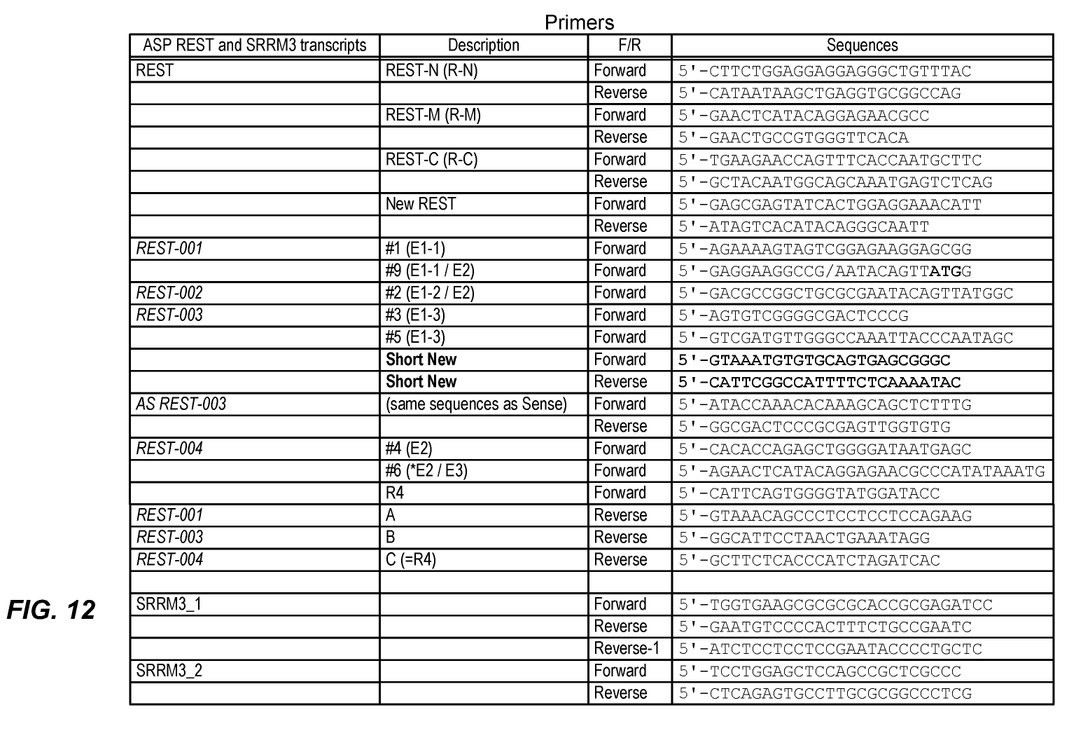

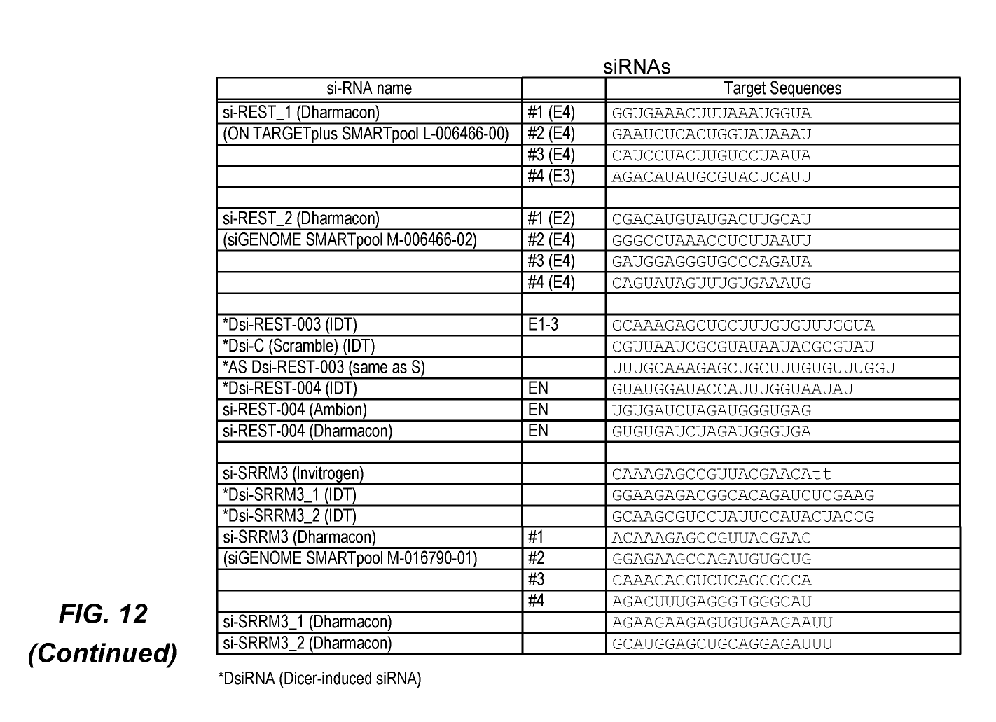

FIG. 12 shows the primers and si-RNAs used.

FIG. 13 shows averaged reads of downregulated gene expression by si-REST-003 treatment from 42 TNBC and 58 controls by the pipeline^{14,15} assay of published RNA-seq data²⁵.

Recent work establishes a strong correlation between cancer invasiveness and the loss of RE1-Silencing Transcription factor (REST), a well-characterized protein best known for suppressing neuronal genes during development⁵⁻⁸. The REST-dependent invasive phenotype is explored in more detail using two breast cancer cell lines: MDA-MB-231, which is strongly invasive, and MCF-7, which is weakly invasive. The invasive potential of each cell line is confirmed using Matrigel invasion chamber assays⁹ (FIG. 4a). In agreement with published studies^{5,6}, REST mRNA and protein levels were higher in MCF-7 relative to MDA-MB-231 cells (FIGS. 4b and 4c).

Next, REST was downregulated in MCF-7 cells using two siRNAs (si-REST_1 and si-REST_2; see FIG. 4d and FIG. 12). Treated cells exhibited increased invasiveness in Matrigel assays (FIG. 4e). Then, wild-type (wt) REST¹⁰ was overexpressed in MDA-MB-231 cells by transfection with REST cDNA and observed decreased Matrigel invasion (FIG. 4f). These data confirm a negative correlation between REST expression and invasiveness^{5,7}.

To identify candidate genes that mediate invasiveness, RNA-sequencing (RNA-seq) analysis (GEO accession# GSE63610) was performed of MCF-7 and MDA-MB-231 cells. Surprisingly, REST levels did not differ significantly between the two cell lines. Instead, an alternatively spliced product (ASP) of REST, REST-003, was found whose expression was low in MCF-7 and high in MDA-MB-231 cells (FIG. 1a, FIG. 5). Due to the complex nature of REST alternative splicing in cancer cells¹¹ as well as the small size of RNA-seq reads, additional experiments were performed to confirm this result and determine its role in invasiveness. Only four REST ASPs are catalogued in the Ensembl Human Genome Browser database (version 75; (http://uswest.ensembl.org/index.html)). The analysis was confined to these forms (REST-001, REST-002, REST-003, and REST-004), which are illustrated schematically in FIG. 1b along with the REST gene (also see FIG. 6 and Supplementary Information). A translation initiation codon is present in Exon 2 (E2). REST-001 and REST-002 produce full-length REST protein but contain different 5′ untranslated regions (UTRs). REST-004 lacks the E2 initiation codon and may thus produce non-coding RNA (ncRNA). REST-003 contains the initiation codon but lacks other parts of the E2 coding sequence. Since no available data identify REST-003 as a protein-coding gene, the structures of the REST ASPs was analyzed with qRT-PCR, using specific primers to distinguish the presence or absence of the E2 initiation codon (FIG. 1b). Primers flanking the E2 initiation codon or the middle part of the coding region (R-N and R-M primer pairs) detected high REST expression in MCF-7 cells and low expression in MDA-MB-231 cells (FIGS. 1c and 1d). A similar result was obtained with primers flanking the E2 initiation codon but confined to sequences present only in REST-003 (5-A primer pair; FIG. 7a). When testing primers that exclude the initiation codon but are specific to REST-003 (3-B primer pair), low expression was observed in MCF-7 cells as well as in other weakly invasive breast cancer cell lines (FIGS. 8a and 8b), and high expression in MDA-MB-231 cells (FIGS. 1c and 1d). Similarly, the invasive bladder cancer cell line, T24/83, expressed REST-003 at higher levels than the non-invasive RT112/84 line (FIGS. 8c and 8d). These results support the RNA-seq data and suggest that only ncRNA derived from REST-003 correlates positively with invasiveness.

Next, the effect of REST modulation on REST-003 expression was established using RNA-seq and qRT-PCR. Both methods indicated enhanced REST-003 expression (>2 fold) following REST downregulation in MCF-7 cells (FIGS. 1a and 1e). Expression of the potential coding RNAs (REST-002, primer pair 2-A or REST-003, primer pair 5-A) decreased relative to control expression (FIG. 7a), a pattern similar to that of REST-001 (FIG. 1e and FIG. 7a). Conversely, overexpression of REST in MDA-MB-231 cells resulted in decreased REST-003 expression (FIG. 1a, FIG. 1f and FIG. 7b). These results suggest that increased expression of REST-003 ncRNA (following loss of REST) may mediate breast cancer cell invasiveness.

To verify this, siRNA specific for REST-003 (si-REST-003) was used. The si-REST-003-treated MDA-MB-231 cells exhibited decreased REST-003 expression (>50%) and reduced Matrigel invasion (>50%) relative to control cells treated with scrambled RNA (si-C; FIGS. 2a and 2b). Treated cells did not show a change in REST-001 expression (FIGS. 2a and 2b). This implicates a primary role for REST-003 in regulating invasiveness that is, at least in part, independent of REST protein expression.

Next, it was questioned how REST downregulation could result in increased REST-003 expression. In neuronal cells, REST transcript is alternatively spliced to produce a REST4 protein, which activates gene expression by competing with REST for RE-1 DNA binding sites¹². This alternative splicing is mediated by a neural-specific serine/arginine (SR) repetitive matrix 4 protein, SRRM4 (also known as nSR100)¹³. No SRRM4 expression was detected by RNA-seq following REST overexpression in MDA-MB-231 cells or in MCF-7 cells treated with si-REST_2 (FIG. 2c). There was, however, a significant change in expression of a related gene, SRRM3 (FIG. 2c), which has no previously documented function. SRRM3 expression increases >2 fold in MCF-7 cells following treatment with si-REST RNAs, though its expression in MCF-7 is much higher than that in MDA-MB-231, as estimated by the standard pipeline analysis^{14,15} (FIG. 2c). This increase was validated using qRT-PCR (FIG. 7c and FIG. 12). In contrast, SRRM3 expression decreased by ˜3 fold (CuffDiff and the pipeline) in MDA-MB-231 cells following REST overexpression (FIG. 1c). These data were confirmed with qRT-PCR (FIG. 7d).

SRRM3 contains a cwf21 domain, suggesting interaction with SR proteins¹⁶. It also contains SR-rich domains scattered throughout its sequence (FIG. 2d). However, it lacks a canonical RNA recognition motif (RRM) thought to be necessary for alternative splicing. SRRM3 may thus be an “SR-related protein”¹⁷ that enhances transcription not by splicing, but in a manner similar to the RSR-2 protein in C. elegans¹⁸. It was hypothesized that increased REST-003 expression and invasiveness were mediated by increased levels of SRRM3 following REST downregulation. To support this notion, when SRRM3 expression is suppressed in MDA-MB-231 cells using siRNA (si-SRRM3), lower SRRM3 and REST-003 expression is observed (FIG. 2e). Importantly, SRRM3 suppression also reduced MDA-MB-231 invasiveness (FIG. 2f). Co-transfection of MDA-MB-231 cells with si-REST-003 and si-REST_2 eliminated the change in REST-003 expression as well as the reduction in invasiveness (FIGS. 2f and 2g), suggesting that SRRM3 regulatory control of REST-003 is likely positioned downstream of REST protein.

REST-003 appears to be expressed as a ˜150-nt-long mid-size non-coding RNA (mncRNA) positioned within the first exon of REST mRNA (FIGS. 1b and 1c). Recent findings reveal many new small- and mid-size ncRNAs that are enriched at the 5′ boundaries of some human genes^{3,4}. The potential presence of a cluster of REST-003 mncRNAs was investigated using northern blot analysis of the 5′ region of REST (E1-3 region; FIG. 3a) and found several ncRNAs derived from this region (FIG. 9). Sequences with a length of ˜70-200 nt are especially enriched in MDA-MB-231 cells and include both sense (S) and anti-sense (AS) sequences (FIGS. 3b and 3c), an expression pattern similar to that of promoter-associated small RNAs (PASRs) that are not yet functionally defined^{3,4}. The ˜75- and ˜87-nt-long REST-003 S and the ˜75-, ˜87-, and ˜135-nt-long REST-003 AS mncRNA sequences were most highly expressed in MDA-MB-231 relative to MCF-7 cells. Since REST modulation affects REST-003 expression (FIG. 1e and 1f), northern analysis was performed in MDA-MB-231 cells overexpressing REST and in MCF-7 cells treated with si-REST_2. Expression of the ˜75- and ˜87-nt REST-003 S and the ˜135-nt REST-003 AS mncRNAs was found to be negatively regulated by REST (FIGS. 3b and 3c, blue boxes).

The levels of the mncRNAs following treatment of MDA-MB-231 cells with si-SRRM3 was also investigated. While expression of the ˜75- and 135-nt REST-003 AS mncRNAs were downregulated, REST-003 S mncRNA was unaffected (FIGS. 3b and 3c, green boxes). Additionally, at least five larger (>200 nt) REST-003 S and AS bands were highly expressed in MCF-7 cells, similar to REST-001 (FIG. 9). Notably, there was no detection of ˜21-23 nt double-stranded RNAs potentially derived from Dicer processing of REST-003. Taken together, the data indicate at least eight RNA variants derived from REST primary transcript (FIG. 9). Three appear to be mncRNAs that likely promote invasiveness (FIGS. 3b and 3c). The other five could potentially code for modified proteins and/or produce other ncRNAs that inhibit invasiveness, similar to REST protein.

Next, a link was sought between REST-003 expression, REST target genes, and pathways related to invasion by performing RNA-seq analysis (GEO accession# GSE63610) of MDA-MB-231 cells treated with si-REST-003 (targeting the E1-3 region). Fifty-six genes from DESeq were differentially expressed in the treated versus control samples (FIG. 3d, FIG. 10a). Ten were downregulated in the treated sample, while the other 46 were upregulated. Six of the downregulated genes (PLEC, SYK, STK35, SLC35B2, CUL4a, and EPCAM) are known to facilitate cancer cell invasion and/or extravasation for metastasis¹⁹⁻²⁴ (FIG. 3d). Additionally, these genes were compared with published RNA-seq data from breast cancer cell lines and tissues²⁵. Four genes (PLEC, ANXA10, EHF, SLC4A, CUL4A) were expressed highly in MDA-MB-231 relative to MCF-7 cells, and three (MAGED1, SYK, EPCAM) were expressed more in triple-negative tissues relative to control reduction mammoplasty tissues (FIG. 3d). The 46 upregulated genes in the treated sample were classified with DAVID functional analysis (FIGS. 10a and 10b) and found more than 20% to be related to immune, defense, wounding, and inflammatory responses (FIG. 10c). Interestingly, neither REST nor its canonical neuronal target genes were affected by knockdown of REST-003 ncRNAs. These results indicate that REST-003 mncRNAs play an important role in cancer cell invasion that appears to be largely independent of REST or REST target gene function.

This is the first time a functional role for REST-003 mncRNA is identified: a positive mediator of invasiveness regulated by SRRM3. This regulatory control appears to be a new functional example of a PASR-like process (FIG. 4c). The findings suggest novel therapeutic approaches for limiting breast cancer invasion and metastasis.

Cell Culture, Transfection, qRT-PCR and Northern Blot

MDA-MB-231 and MCF-7 cancer cell lines were obtained and cultured as described previously²⁶. Lipofectamine 2000 (Invitrogen) was used for all transfection experiments unless otherwise specified. For siRNA transfection, DharmaFect (Thermo Scientific) or RNAiMax (Invitrogen) was used according to the manufacturer's instructions. siRNA sequences are provided in FIG. 12.

For qRT-PCR, cDNAs were made from the total RNAs treated with DNase as described in a previous study²⁷. Gene-specific q-PCR primer or probe sets (FIG. 12) for human genes, GAPDH, CyclophilinA, and Actin, and equivalent amounts of cDNA generated as a template were used for qRT-PCR. Reactions were performed for each sample using SSoFast EvaGreen Supermix (Bio-Rad) or TaqMan Universal PCR Master Mix (Invitrogen) with a CFX-96 system (Bio-Rad). For each sample, expression of marker genes was normalized to GAPDH, CyclophilinA or Actin [mean normalized expression (MNE)]. MNEs were normalized to control samples to present relative expression. Northern blots were performed as described previously²⁸.

Matrigel Invasion Assay

Invasion was measured using BD BioCoat Matrigel Invasion Chambers (BD Biosciences) according to the manufacturer's instructions, as described in a previous study²⁶.

Library Preparation, Sequencing, and Analysis of RNA-Seq

Ribosomal RNA (rRNA) was depleted from 100 ng of total RNA using a Ribo-Zero Magnetic Gold kit (Epicentre) according to the manufacturer's protocol. Libraries from rRNA-depleted samples were prepared using a TruSeq RNA Sample Preparation kit v2 (Illumina) following the recommended protocol starting from the RNA fragmentation stage. Purification of polyadenylated RNA was omitted. Libraries were pooled (4 samples per pool), clustered on cBOT (Illumina), and sequenced on HiSeq2000, each pool in one lane. Single-end 100 bp reads were performed. Reads were mapped using TopHat followed by data analysis using Cufflinks and Cuffdiff software, as well as the pipeline^{14,15}.

### Expression Profiling by High Throughput Sequencing

A negative correlation of invasiveness with REST expression is reported. In addition, one alternatively spliced product (ASP) of REST, REST-003, shows a positive correlation with invasiveness. REST has a well-established role in regulating transcription of genes important for neuronal development. Its role in cancer, though significant, is less well understood. It is desired to investigate the effect of REST on invasive phenotype. In order to do so, REST is downregulated by siRNA treatment in weakly invasive MCF-7 breast cancer cells in which REST is expressed highly: 1) si-GAPDH (control), two si-RESTs (2) si-REST_1 and 3) si-REST_2). Conversely, REST is overexpressed by transfection of wt-REST cDNA in highly invasive MDA-MB-231 cells in which REST is expressed at the low level: 4) EGFP (control), 5) mt-REST (another control) and 6) wt-REST.

REST (repressor element-1 (RE-1) silencing transcription factor) contains a DNA-binding domain that is localized within eight zinc fingers and two repressor domains located at the N-terminal and C-terminal, respectively. REST suppresses expression of neural-specific genes. mt-REST lacks two repressor domains, so it can be used as a control for wt-REST. In contrast, REST-003 is one of alternatively spliced products (ASPs) of REST.

### Expression Profiling by High Throughput Sequencing

Fifty six genes from DESeq were differentially expressed in the treated versus control samples. More than 20% were related to immune, defense, wounding and inflammatory responses.

Downregulation of REST using two siRNAs in MCF-7 cells or overexpression of REST with wt- or mt-REST cDNA in MDA-MB-231 cells. Downregulation of REST-003 using siRNAs in MDA-MB-231 cells; siRNA against REST-003 was used, as REST-003 may control invasiveness. si-Control (scramble) or si-REST-003 were transfected in MDA-MB-231: duplicate of both (total 4 samples).

### Treatment Protocol

Lipofectamine 2000 (Invitrogen) was used for mt- or wt-REST cDNA plasmid transfection experiments. For siRNA transfection, DharmaFect (Thermo Scientific) or RNAiMax (Invitrogen) was used according to the manufacturer's instructions.

### Growth Protocol

MDA-MB-231 and MCF-7 cancer cell lines were maintained in a modified complete medium (RPMI, 10% FBS, 10 mM HEPES, 2 mM L-glutamine, 1 mM sodium-pyruvate, 0.05 mM 2-mercaptoethanol, 11 mM D-glucose).

### Extraction Protocol

Total RNAs were isolated from cell lines using Trizol (Invitrogen) and digested with Turbo DNase (Ambion) to remove genomic DNA, according to the manufacturer's instructions. Libraries from rRNA-depleted samples were prepared using a TruSeq RNA Sample Preparation kit v2 (Illumina) following the recommended protocol starting from the RNA fragmentation stage. Purification of polyadenylated RNA was omitted. Libraries were pooled (4 samples per pool), clustered on cBOT (Illumina), and sequenced on HiSeq2000, each pool in one lane. Single-end 100 bp reads were performed. Reads were mapped using TopHat followed by data analysis using Cufflinks and Cuffdiff software, as well as the own pipeline.

TABLE 1
Part 1 of 2: REST Primers

| ASP REST | | | |
|---|---|---|---|
| and SRRM3 | | | |
| Transcripts | Description | F/R | Sequences |
| REST | REST-N (R-N) | Forward | 5′-CTTCTGGAGGAGGAGGGCTGTTTAC |
| | | Reverse | 5′-CATAATAAGCTGAGGTGCGGCCAG |
| | REST-M (R-M) | Forward | 5′-GAACTCATACAGGAGAACGCC |
| | | Reverse | 5′-GAACTGCCGTGGGTTCACA |
| | REST-C (R-C) | Forward | 5′-TGAAGAACCAGTTTCACCAATGCTTC |
| | | Reverse | 5′-GCTACAATGGCAGCAAATGAGTCTCAG |
| | new REST | Forward | 5′-GAGCGAGTATCACTGGAGGAAACATT |
| | | Reverse | 5′-ATAGTCACATACAGGGCAATT |
| REST-001 | #1(E1-1) | Forward | 5′-AGAAAAGTAGTCGGAGAAGGAGCGG |
| | #9(E1-1/E2) | Forward | 5′-GAGGAAGGCCG/AATACAGTTATGG |
| REST-002 | #2(E1-2/E2) | Forward | 5′-GACGCCGGCTGCGCG AATACAGTTATGGC |
| REST-003 | #3(E1-3) | Forward | 5′-AGTGTCGGGGCGACTCCCG |
| | #5(E1-3) | Forward | 5′-GTCGATGTTGGGCCAAATTACCCAATAGC |
| | Short New | Forward | 5′-GTAAATGTGTGCAGTGAGCGGGC |
| | Short New | Reverse | 5′-CATTCGGCCATTTTCTCAAAATAC |
| AS REST-003 | (same sequences as Sense) | Forward | 5′-ATACCAAACACAAAGCAGCTCTTTG |
| | | Reverse | 5′-GGCGACTCCCGCGAGTTGGTGTG |
| REST-004 | #4(E2) | Forward | 5′-CACACCAGAGCTGGGGATAATGAGC |
| | #6(*E2/E3) | Forward | 5′-AGAACTCATACAGGAGAACGCCCATATAAATG |
| | R4 | Forward | 5′-CATTCAGTGGGGTATGGATACC |
| REST-001 | A | Reverse | 5′-GTAAACAGCCCTCCTCCTCCAGAAG |
| REST-003 | B | Reverse | 5′-GGCATTCCTAACTGAAATAGG |
| REST-004 | C (=R4) | Reverse | 5′-GCTTCTCACCCATCTAGATCAC |
| SRRM3_1 | | Forward | 5′-TGGTGAAGCGCGCGCACCGCGAGATCC |
| | | Reverse | 5′-GAATGTCCCCACTTTCTGCCGAATC |
| | | Reverse-1 | 5′-ATCTCCTCCTCCGAATACCCCTGCTC |
| SRRM3_2 | | Forward | 5′-TCCTGGAGCTCCAGCCGCTCGCCC |
| | | Reverse | 5′-CTCAGAGTGCCTTGCGCGGCCCTCG |

Part 2 of 2: siRNA Sequences

| si-RNA name | | Target Sequences |
|---|---|---|
| si-REST_1 (Dharmacon) | #1(E4) | GGUGAAACUUUAAAUGGUA |
| (ON-TARGETplus SMARTpool L-006466-00) | #2(E4) | GAAUCUCACUGGUAUAAAU |
| | #3(E4) | CAUCCUACUUGUCCUAAUA |
| | #4(E3) | AGACAUAUGCGUACUCAUU |
| si-REST_2 (Dharmacon) | #1(E2) | CGACAUGUAUGACUUGCAU |
| (siGENOME SMARTpool M-006466-02) | #2(E4) | GGGCCUAAACCUCUUAAUU |
| | #3(E4) | GAUGGAGGGUGCCCAGAUA |
| | #4(E4) | CAGUAUAGUUUGUGAAAUG |
| *Dsi-REST-003 (IDT) | E1-3 | GCAAAGAGCUGCUUUGUGUUUGGUA |
| *Dsi-C (Scramble) (IDT) | | CGUUAAUCGCGUAUAAUACGCGUAU |
| *AS Dsi-REST-003 (same as S) | | UUUGCAAAGAGCUGCUUUGUGUUUGGU |
| *Dsi-REST-004 (IDT) | EN | GUAUGGAUACCAUUUGGUAAUAU |
| si-REST-004 (Ambion) | EN | UGUGAUCUAGAUGGGUGAG |
| si-REST-004 (Dharmacon) | EN | GUGUGAUCUAGAUGGGUGA |
| si-SRRM3 (Ambion) | | CAAAGAGCCGUUACGAACAtt |
| *Dsi-SRRM3_1 | (IDT) | GGAAGAGACGGCACAGAUCUCGAAG |
| *Dsi-SRRM3_2 | (IDT) | GCAAGCGUCCUAUUCCAUACUACCG |
| si-SRRM3 (Dharmacon) | #1 | ACAAAGAGCCGUUACGAAC |
| (siGENOME SMARTpool M-016790-01) | #2 | GGAGAAGCCAGAUGUGCUG |
| | #3 | CAAAGAGGUCUCAGGGCCA |
| | #4 | AGACUUUGAGGGTGGGCAU |
| si-SRRM3_1 (Dharmacon) | | AGAAGAAGAGUGUGAAGAAUU |
| si-SRRM3_2 (Dharmacon) | | GCAUGGAGCUGCAGGAGAUUU |

*DsiRNA (Dicer-induced siRNA)

RNA-seq generated during this study has been deposited in GEO under accession number GSE63610, which is incorporated herein by reference.

The APPENDIX which follows provides additional information about the present invention.

### REFERENCES

The following references are each relied upon and incorporated herein in their entirety.
1 Ballas, N. & Mandel, G. The many faces of REST oversee epigenetic programming of neuronal genes. Current opinion in neurobiology 15, 500-506, doi:10.1016/j.conb.2005.08.015 (2005).
2 Negrini, S., Prada, I., D'Alessandro, R. & Meldolesi, J. REST: an oncogene or a tumor suppressor? Trends in cell biology 23, 289-295, doi:10.1016/j.tcb.2013.01.006 (2013).
3 Djebali, S. et al. Landscape of transcription in human cells. Nature 489, 101-108, doi:10.1038/nature 11233 (2012).
4 Kapranov, P. et al. RNA maps reveal new RNA classes and a possible function for pervasive transcription. Science 316, 1484-1488, doi:10.1126/science.1138341 (2007).
5 Reddy, B. Y., Greco, S. J., Patel, P. S., Trzaska, K. A. & Rameshwar, P. RE-1-silencing transcription factor shows tumor-suppressor functions and negatively regulates the oncogenic TAC1 in breast cancer cells. Proc Natl Acad Sci USA 106, 4408-4413, doi:10.1073/pnas.0809130106 (2009).
6 Lv, H. et al. Expression and functions of the repressor element 1 (RE-1)-silencing transcription factor (REST) in breast cancer. Journal of cellular biochemistry 110, 968-974, doi:10.1002/jcb.22610 (2010).
7 Wagoner, M. P. et al. The transcription factor REST is lost in aggressive breast cancer. PLoS genetics 6, e1000979, doi:10.1371/journal.pgen.1000979 (2010).
8 Shimojo, M., Shudo, Y., Ikeda, M., Kobashi, T. & Ito, S. Small cell lung cancer-specific isoform of RE1-silencing transcription factor (REST) is regulated by neural-specific Ser/Arg repeat-related protein of 100 kDa (nSR100). Molecular cancer research: MCR, doi:10.1158/1541-7786.MCR-13-0269 (2013).
9 Albini, A. et al. A rapid in vitro assay for quantitating the invasive potential of tumor cells. Cancer research 47, 3239-3245 (1987).
10 Tapia-Ramirez, J., Eggen, B. J., Peral-Rubio, M. J., Toledo-Aral, J. J. & Mandel, G. A single zinc finger motif in the silencing factor REST represses the neural-specific type II sodium channel promoter. Proc Natl Acad Sci USA 94, 1177-1182 (1997).
11 Chen, G. L. & Miller, G. M. Extensive alternative splicing of the repressor element silencing transcription factor linked to cancer. PLoS One 8, e62217, doi:10.1371/journal.pone.0062217 (2013).
12 Shimojo, M., Paquette, A. J., Anderson, D. J. & Hersh, L. B. Protein kinase A regulates cholinergic gene expression in PC12 cells: REST4 silences the silencing activity of neuron-restrictive silencer factor/REST. Molecular and cellular biology 19, 6788-6795 (1999).
13 Raj, B. et al. Cross-regulation between an alternative splicing activator and a transcription repressor controls neurogenesis. Molecular cell 43, 843-850, doi:10.1016/j.molce1.2011.08.014 (2011).
14 Souaiaia, T., Frazier, Z. & Chen, T. ComB: SNP calling and mapping analysis for color and nucleotide space platforms. Journal of computational biology: a journal of computational molecular cell biology 18, 795-807, doi:10.1089/cmb.2011.0027 (2011).
15 Wang, Y. et al. RseqFlow: workflows for RNA-Seq data analysis. Bioinformatics 27, 2598-2600, doi:10.1093/bioinformatics/btr441 (2011).
16 Grainger, R. J., Barrass, J. D., Jacquier, A., Rain, J. C. & Beggs, J. D. Physical and genetic interactions of yeast Cwc21p, an ortholog of human SRm300/SRRM2, suggest a role at the catalytic center of the spliceosome. Rna 15, 2161-2173, doi:10.1261/rna.1908309 (2009).
17 Leoutsakou, T., Talieri, M. & Scorilas, A. Prognostic significance of the expression of SR-A1, encoding a novel SR-related CTD-associated factor, in breast cancer. Biological chemistry 387, 1613-1618, doi:10.1515/BC.2006.201 (2006).
18 Fontrodona, L. et al. RSR-2, the Caenorhabditis elegans ortholog of human spliceosomal component SRm300/SRRM2, regulates development by influencing the transcriptional machinery. PLoS genetics 9, e1003543, doi:10.1371/journal.pgen.1003543 (2013).
19 Banyard, J. et al. Identification of genes regulating migration and invasion using a new model of metastatic prostate cancer. BMC cancer 14, 387, doi:10.1186/1471-2407-14-387 (2014).
20 Saucedo-Cuevas, L. P. et al. CUL4A contributes to the biology of basal-like breast tumors through modulation of cell growth and antitumor immune response. Oncotarget 5, 2330-2343 (2014).
21 Goyal, P., Behring, A., Kumar, A. & Siess, W. STK35L1 associates with nuclear actin and regulates cell cycle and migration of endothelial cells. PLoS One 6, e16249, doi:10.1371/journal.pone.0016249 (2011).
22 Kamiyama, S. et al. Expression and the role of 3′-phosphoadenosine 5′-phosphosulfate transporters in human colorectal carcinoma. Glycobiology 21, 235-246, doi:10.1093/glycob/cwq154 (2011).
23 Krisenko, M. O., Cartagena, A., Raman, A. & Geahlen, R. L. Nanomechanical Property Maps of Breast Cancer Cells As Determined by Multiharmonic Atomic Force Microscopy Reveal Syk-Dependent Changes in Microtubule Stability Mediated by MAP1B. Biochemistry, doi:10.1021/bi500325n (2014).
24 Sugiyama, N. et al. In vivo selection of high-metastatic subline of bladder cancer cell and its characterization. Oncology research 20, 289-295 (2013).
25 Varley, K. E. et al. Recurrent read-through fusion transcripts in breast cancer. Breast Cancer Res Treat 146, 287-297, doi:10.1007/s10549-014-3019-2 (2014).
26 Hwang, J. Y. et al. Investigating contactless high frequency ultrasound microbeam stimulation for determination of invasion potential of breast cancer cells. Biotechnology and bioengineering, doi:10.1002/bit.24923 (2013).
27 Lee, N. S. et al. A novel dual-color reporter for identifying insulin-producing beta-cells and classifying heterogeneity of insulinoma cell lines. PLoS One 7, e35521, doi:10.1371/journal.pone.0035521 (2012).
28 Lee, N. S. et al. Expression of small interfering RNAs targeted against HIV-1 rev transcripts in human cells. Nature biotechnology 20, 500-505, doi:10.1038/nbt0502-500 (2002).
29 Chong, J. A. et al. REST: a mammalian silencer protein that restricts sodium channel gene expression to neurons. Cell 80, 949-957 (1995).

What is claimed is:

1. A method comprising:
(i) assaying a subject's tissue sample for an expression level of one or more oligonucleotide sequences, wherein each of the one or more oligonucleotide sequences independently comprises:
a. at least 80% homology to or at least 80% of the nucleobases or any combination thereof of at least a portion of a (RE-1)-Silencing transcription factor 003 (REST-003) sequence or fragment thereof,
b. at least 80% homology to or at least 80% of the nucleobases or any combination thereof of at least a portion of one or more REST-003-mediated oligonucleotide sequences or fragments thereof, or
c. any combination thereof, and
(ii) comparing the expression level obtained in (i) to an expression level of the one or more oligonucleotide sequences of a control,
wherein the expression level of the one or more oligonucleotide sequences of the control is a reference value obtained from a database comprising an average expression level for at least one of the one or more oligonucleotide sequences, wherein the average expression level is obtained from: at least 1, at least 5, at least 10, at least 15, or at least 20 non-cancerous, non-tumorous, or non-cancerous and non-tumorous tissue samples.

2. The method of claim 1, further comprising, determining a likelihood of metastasis, a risk of tumor or cancer occurrence, an invasion potential, an effectiveness of a cancer or tumor treatment, an effectiveness of a drug, a longitudinal course of a cancer or tumor treatment regime, or any combination thereof, in the subject based on the comparing.

3. The method of claim 2, wherein the method is for evaluating the tissue sample of the subject to determine the likelihood of metastasis, the risk of tumor or cancer occurrence, the invasion potential, the effectiveness of a cancer or tumor treatment, the effectiveness of a drug, the longitudinal course of a cancer or tumor treatment regime, or any combination thereof in the subject.

4. (canceled)

5. The method of claim 1, wherein each of the one or more oligonucleotide sequences independently comprises (i) at least 95% homology to or at least 95% of the nucleobases or any combination thereof of at least a portion of the REST-003 sequence or fragment thereof, (ii) at least 95% homology to or at least 95% of the nucleobases or any combination thereof of at least a portion of the one or more REST-003-mediated oligonucleotide sequences or fragments thereof, or (iii) any combination thereof.

6. The method of claim 1, wherein the control is a non-cancerous tissue sample, a non-tumor tissue sample, or a combination thereof.

7. The method of claim 1, wherein the tissue sample is an excised tissue, a biopsy, a fine needle aspirate, a cytology specimen, a tissue washing, or any combination thereof.

8.-9. (canceled)

10. The method of claim 1, wherein the tissue sample is a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

11. The method of claim 2, further comprising determining a risk of cancer occurrence, wherein the risk of cancer occurrence is a risk of breast cancer occurrence or a bladder cancer occurrence.

12. The method of claim 2, further comprising determining a risk of tumor occurrence, wherein the risk of tumor occurrence is a breast tumor occurrence or a bladder tumor occurrence.

13. The method of claim 2, further comprising determining a risk of tumor or cancer occurrence, wherein the risk of tumor or cancer occurrence is a risk of an occurrence of an adrenal cortical cancer, anal cancer, aplastic anemia, bile duct cancer, bladder cancer, bone cancer, bone metastasis, central nervous system (CNS) cancer, peripheral nervous system (PNS) cancer, breast cancer, Castleman's disease, cervical cancer, childhood on-Hodgkin's lymphoma, lymphoma, colon and rectum cancer, endometrial cancer, esophagus cancer, Ewing's sarcoma, eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gfand cancer, sarcoma, melanoma skin cancer, non-melanoma skin cancer, stomach cancer, testicular cancer, thymus cancer, uterine cancer, vaginal cancer, vulvar cancer, Waldenstrom's macrogiobulinemia, or any combination thereof.

14.-20. (canceled)

21. The method of claim 1, wherein prior to (i), tumor cells, cancer cells, or a combination thereof are (a) identified in the tissue sample, (b) are enriched in the sample, or (c) a combination thereof.

22.-36. (canceled)

37. The method of claim 1, wherein the one or more oligonucleotide sequences independently comprise a sequence from an E1-3 region to an E2 region of the REST-003 sequence or fragment thereof.

38.-47. (canceled)

48. The method of claim 1, wherein the one or more REST-003-mediated oligonucleotide sequences or fragments thereof comprise less than about 20 different genes.

49. The method of claim 1, wherein each of the one or more oligonucleotide sequences independently comprises at least 80% homology to or at least 80% of the nucleobases or any combination thereof of at least a portion of one or more REST-003-mediated oligonucleotides sequences or fragments thereof, and wherein the one or more oligonucleotides comprise a sequence encoding a gene selected from the group consisting of PLEC, MAGED1, SYK, STK35, ANXA10, EHF, SLC35B2, CUL4A, EPCAM, MTMR4, fragments thereof, and any combinations thereof.

50. The method of claim 49, wherein an expression level of the one or more REST-003-mediated oligonucleotide sequences or fragments thereof that is at least about 10% higher than the expression level of the control indicates a likelihood of metastasis, a risk of tumor or cancer occurrence, or a combination thereof, in the subject.

51. The method of claim 50, wherein the at least about 10% higher expression level of the one or more REST-003-mediated oligonucleotide sequences or fragments thereof is subtracted from the expression level of the control, a result of which is divided by the expression level of the control and multiplied by 100.

52. The method of claim 1, wherein the one or more REST-003-mediated oligonucleotide sequences or fragments thereof comprise from about 10 different genes to about 70 different genes.

53. The method of claim 1, wherein each of the one or more oligonucleotide sequences independently comprises at least 80% homology to or at least 80% of the nucleobases or any combination thereof of at least a portion of one or more REST-003-mediated oligonucleotides sequences or fragments thereof, and wherein the one or more oligonucleotides comprise a sequence encoding a gene selected from the group consisting of IFNL1, CXCL10, IFNB1, CXCL11, CCR1, GBP5, APOL3, GBP4, CIS, CASP1, XAF1, CCL5, IDO1, IRG1, GBP1, TNFSF10, CD274, RTP4, IFIT2, TFPI2, APOL1, GBP IP 1, BST2, IFIT3, TGFBI, TRIM22, PSAT1, RSAD2, CEACAM1, GBP2, TMEM171, IL8, TLR3, CBX1, OASL, SERPINE1, MMP13, IL1B, HERC5, FNDC3A, CMPK2, ARL6IP1, PGAM1, TAP1, PMAIP1, IL6, fragments thereof, and any combination thereof.

54. The method of claim 1, wherein an expression level of the one or more REST-003-mediated oligonucleotide sequences or fragments thereof that is at least about 0.5% lower than an expression level of the control indicates a likelihood of metastasis, a risk of tumor or cancer occurrence, or a combination thereof, in the subject.

55.-85. (canceled)

86. A method of treating a subject, comprising administrating a small molecule, an antibody or fragment thereof, an siRNA, an aptamer, or any combination thereof to the subject, wherein the small molecule, the antibody or fragment thereof, the siRNA, the aptamer, or any combination thereof binds to at least a portion of the REST-003 sequence or fragment thereof, and wherein the subject is a cancer patient, a tumor patient, or a cancer and tumor patient.

87.-110. (canceled)


SEQUENCE LISTING (69 sequences)

SEQ ID NO: 1 - DNA, 19 bp, Artificial Sequence
  Description of Artificial Sequence Synthetic primer
agtgtcggggcgactcccg

SEQ ID NO: 2 - DNA, 21 bp, Artificial Sequence
  Description of Artificial Sequence Synthetic primer
ggcattcctaactgaaatagg

SEQ ID NO: 3 - DNA, 27 bp, Artificial Sequence
  Description of Artificial Sequence Synthetic primer
tggtgaagcgcgcgcaccgcgagatcc

SEQ ID NO: 4 - DNA, 25 bp, Artificial Sequence
  Description of Artificial Sequence Synthetic primer
gaatgtccccactttctgccgaatc

SEQ ID NO: 5 - DNA, 24 bp, Artificial Sequence
  Description of Artificial Sequence Synthetic primer
tcctggagctccagccgctcgccc

SEQ ID NO: 6 - DNA, 25 bp, Artificial Sequence
  Description of Artificial Sequence Synthetic primer
ctcagagtgccttgcgcggccctcg

SEQ ID NO: 7 - DNA, 29 bp, Artificial Sequence
  Description of Artificial Sequence Synthetic primer
gtcgatgttgggccaaattacccaatagc

SEQ ID NO: 8 - DNA, 23 bp, Artificial Sequence
  Description of Artificial Sequence Synthetic primer
gtaaatgtgtgcagtgagcgggc

SEQ ID NO: 9 - DNA, 24 bp, Artificial Sequence
  Description of Artificial Sequence Synthetic primer
cattcggccattttctcaaaatac

SEQ ID NO: 10 - DNA, 25 bp, Artificial Sequence
  Description of Artificial Sequence Synthetic primer
ataccaaacacaaagcagctctttg

SEQ ID NO: 11 - DNA, 23 bp, Artificial Sequence
  Description of Artificial Sequence Synthetic primer
ggcgactcccgcgagttggtgtg

SEQ ID NO: 12 - RNA, 25 nt, Artificial Sequence
  Description of Artificial Sequence Synthetic oligonucleotide
gcaaagagcugcuuuguguuuggua

SEQ ID NO: 13 - RNA, 27 nt, Artificial Sequence
  Description of Artificial Sequence Synthetic oligonucleotide
uuugcaaagagcugcuuuguguuuggu

SEQ ID NO: 14 - DNA, 25 bp, Artificial Sequence
  Description of Artificial Sequence Synthetic primer
cttctggaggaggagggctgtttac

SEQ ID NO: 15 - DNA, 24 bp, Artificial Sequence
  Description of Artificial Sequence Synthetic primer
cataataagctgaggtgcggccag

SEQ ID NO: 16 - DNA, 21 bp, Artificial Sequence
  Description of Artificial Sequence Synthetic primer
gaactcatacaggagaacgcc

SEQ ID NO: 17 - DNA, 19 bp, Artificial Sequence
  Description of Artificial Sequence Synthetic primer
gaactgccgtgggttcaca

SEQ ID NO: 18 - DNA, 26 bp, Artificial Sequence
  Description of Artificial Sequence Synthetic primer
tgaagaaccagtttcaccaatgcttc

SEQ ID NO: 19 - DNA, 27 bp, Artificial Sequence
  Description of Artificial Sequence Synthetic primer
gctacaatggcagcaaatgagtctcag

SEQ ID NO: 20 - DNA, 26 bp, Artificial Sequence
  Description of Artificial Sequence Synthetic primer
gagcgagtatcactggaggaaacatt

SEQ ID NO: 21 - DNA, 21 bp, Artificial Sequence
  Description of Artificial Sequence Synthetic primer
atagtcacatacagggcaatt

SEQ ID NO: 22 - DNA, 25 bp, Artificial Sequence
  Description of Artificial Sequence Synthetic primer
agaaaagtagtcggagaaggagcgg

SEQ ID NO: 23 - DNA, 24 bp, Artificial Sequence
  Description of Artificial Sequence Synthetic primer
gaggaaggccgaatacagttatgg

SEQ ID NO: 24 - DNA, 29 bp, Artificial Sequence
  Description of Artificial Sequence Synthetic primer
gacgccggctgcgcgaatacagttatggc

SEQ ID NO: 25 - DNA, 25 bp, Artificial Sequence
  Description of Artificial Sequence Synthetic primer
cacaccagagctggggataatgagc

SEQ ID NO: 26 - DNA, 32 bp, Artificial Sequence
  Description of Artificial Sequence Synthetic primer
agaactcatacaggagaacgcccatataaatg

SEQ ID NO: 27 - DNA, 22 bp, Artificial Sequence
  Description of Artificial Sequence Synthetic primer
cattcagtggggtatggatacc

SEQ ID NO: 28 - DNA, 25 bp, Artificial Sequence
  Description of Artificial Sequence Synthetic primer
gtaaacagccctcctcctccagaag

SEQ ID NO: 29 - DNA, 22 bp, Artificial Sequence
  Description of Artificial Sequence Synthetic primer
gcttctcacccatctagatcac

SEQ ID NO: 30 - DNA, 26 bp, Artificial Sequence
  Description of Artificial Sequence Synthetic primer
atctcctcctccgaatacccctgctc

SEQ ID NO: 31 - RNA, 19 nt, Artificial Sequence
  Description of Artificial Sequence Synthetic oligonucleotide
ggugaaacuuuaaauggua

SEQ ID NO: 32 - RNA, 19 nt, Artificial Sequence
  Description of Artificial Sequence Synthetic oligonucleotide
gaaucucacugguauaaau

SEQ ID NO: 33 - RNA, 19 nt, Artificial Sequence
  Description of Artificial Sequence Synthetic oligonucleotide
cauccuacuuguccuaaua

SEQ ID NO: 34 - RNA, 19 nt, Artificial Sequence
  Description of Artificial Sequence Synthetic oligonucleotide
agacauaugcguacucauu

SEQ ID NO: 35 - RNA, 19 nt, Artificial Sequence
  Description of Artificial Sequence Synthetic oligonucleotide
cgacauguaugacuugcau

SEQ ID NO: 36 - RNA, 19 nt, Artificial Sequence
  Description of Artificial Sequence Synthetic oligonucleotide
gggccuaaaccucuuaauu

SEQ ID NO: 37 - RNA, 19 nt, Artificial Sequence
  Description of Artificial Sequence Synthetic oligonucleotide
gauggagggugcccagaua

SEQ ID NO: 38 - RNA, 19 nt, Artificial Sequence
  Description of Artificial Sequence Synthetic oligonucleotide
caguauaguuugugaaaug

SEQ ID NO: 39 - RNA, 25 nt, Artificial Sequence
  Description of Artificial Sequence Synthetic oligonucleotide
cguuaaucgcguauaauacgcguau

SEQ ID NO: 40 - RNA, 23 nt, Artificial Sequence
  Description of Artificial Sequence Synthetic oligonucleotide
guauggauaccauuugguaauau

SEQ ID NO: 41 - RNA, 19 nt, Artificial Sequence
  Description of Artificial Sequence Synthetic oligonucleotide
ugugaucuagaugggugag

SEQ ID NO: 42 - RNA, 19 nt, Artificial Sequence
  Description of Artificial Sequence Synthetic oligonucleotide
gugugaucuagauggguga

SEQ ID NO: 43 - DNA, 21 bp, Artificial Sequence
  Description of Artificial Sequence Synthetic oligonucleotide
  Description of Combined DNA/RNA Molecule Synthetic oligonucleotide
caaagagccguuacgaacatt

SEQ ID NO: 44 - RNA, 25 nt, Artificial Sequence
  Description of Artificial Sequence Synthetic oligonucleotide
ggaagagacggcacagaucucgaag

SEQ ID NO: 45 - RNA, 25 nt, Artificial Sequence
  Description of Artificial Sequence Synthetic oligonucleotide
gcaagcguccuauuccauacuaccg

SEQ ID NO: 46 - RNA, 19 nt, Artificial Sequence
  Description of Artificial Sequence Synthetic oligonucleotide
acaaagagccguuacgaac

SEQ ID NO: 47 - RNA, 19 nt, Artificial Sequence
  Description of Artificial Sequence Synthetic oligonucleotide
ggagaagccagaugugcug

SEQ ID NO: 48 - RNA, 19 nt, Artificial Sequence
  Description of Artificial Sequence Synthetic oligonucleotide
caaagaggucucagggcca

SEQ ID NO: 49 - DNA, 19 bp, Artificial Sequence
  Description of Artificial Sequence Synthetic oligonucleotide
  Description of Combined DNA/RNA Molecule Synthetic oligonucleotide
agacuuugagggtgggcau

SEQ ID NO: 50 - RNA, 21 nt, Artificial Sequence
  Description of Artificial Sequence Synthetic oligonucleotide
agaagaagagugugaagaauu

SEQ ID NO: 51 - RNA, 21 nt, Artificial Sequence
  Description of Artificial Sequence Synthetic oligonucleotide
gcauggagcugcaggagauuu

SEQ ID NO: 52 - protein, 46 aa, Unknown
  Description of Unknown SRRM3 polypeptide
LDHERKRRVELKCMELQEMMEEQGYSEEEIRQKVGTFRQMLMEKEG

SEQ ID NO: 53 - protein, 46 aa, Saccharomyces cerevisiae
LEHERKREIEVKCLELRDKLEEQGLSEEEIEEKVDELRKKLLEELE

SEQ ID NO: 54 - protein, 46 aa, Homo sapiens
LDHERKRRVELRCLELEEMMEEQGYEEQQIQEKVATFRLMLLEKDV

SEQ ID NO: 55 - DNA, 305 bp, Artificial Sequence
  Description of Artificial Sequence Synthetic polynucleotide
gcagcgtcctgtgttggaatgtgcggctgccgcgagctcgcggcgcagcagcggagcgagcgccgccgaggcccggggccccagaccctggcggcggctgccgcagccgagacggcagggcgaggcccggaggcctgagcaccctctgcagccccactcctgggccttcttggtccacgacggccccagcacccaactttaccaccctcccccacctctcccccgaaactccagcaacaaagaaaagtagtcggagaaggagcggcgactcagggtcgcccgcccctcctcaccgaggaaggccg

SEQ ID NO: 56 - DNA, 907 bp, Artificial Sequence
  Description of Artificial Sequence Synthetic polynucleotide
aatacagttatggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacag

SEQ ID NO: 57 - DNA, 84 bp, Artificial Sequence
  Description of Artificial Sequence Synthetic oligonucleotide
gagaacgcccatataaatgtgaactttgtccttactcaagttctcagaagactcatctaactagacatatgcgtactcattcag

SEQ ID NO: 58 - DNA, 6004 bp, Artificial Sequence
  Description of Artificial Sequence Synthetic polynucleotide
gtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcaattcagaaaaattcagtaaaactaagaaaagcaaaaggaagctggaagttgacagccattctttacatggtcctgtgaatgatgaggaatcttcaacaaaaaagaaaaagaaggtagaaagcaaatccaaaaataatagtcaggaagtgccaaagggtgacagcaaagtggaggagaataaaaagcaaaatacttgcatgaaaaaaagtacaaagaagaaaactctgaaaaataaatcaagtaagaaaagcagtaagcctcctcagaaggaacctgttgagaagggatctgctcagatggaccctcctcagatggggcctgctcccacagaggcggttcagaaggggcccgttcaggtggagccgccacctcccatggagcatgctcagatggagggtgcccagatacggcctgctcctgacgagcctgttcagatggaggtggttcaggaggggcctgctcagaaggagctgctgcctcccgtggagcctgctcagatggtgggtgcccaaattgtacttgctcacatggagctgcctcctcccatggagactgctcagacggaggttgcccaaatggggcctgctcccatggaacctgctcagatggaggttgcccaggtagaatctgctcccatgcaggtggtccagaaggagcctgttcagatggagctgtctcctcccatggaggtggtccagaaggagcctgttcagatagagctgtctcctcccatggaggtggtccagaaggaacctgttaagatagagctgtctcctcccatagaggtggtccagaaggagcctgttcagatggagttgtctcctcccatgggggtggttcagaaggagcctgctcagagggagccacctcctcccagagagcctccccttcacatggagccaatttccaaaaagcctcctctccgaaaagataaaaaggaaaagtctaacatgcagagtgaaagggcacggaaggagcaagtccttattgaagttggcttagtgcctgttaaagatagctggcttctaaaggaaagtgtaagcacagaggatctctcaccaccatcaccaccactgccaaaggaaaatttaagagaagaggcatcaggagaccaaaaattactcaacacaggtgaaggaaataaagaagcccctcttcagaaagtaggagcagaagaggcagatgagagcctacctggtcttgctgctaatatcaacgaatctacccatatttcatcctctggacaaaacttgaatacgccagagggtgaaactttaaatggtaaacatcagactgacagtatagtttgtgaaatgaaaatggacactgatcagaacacaagagagaatctcactggtataaattcaacagttgaagaaccagtttcaccaatgcttcccccttcagcagtagaagaacgtgaagcagtgtccaaaactgcactggcatcacctcctgctacaatggcagcaaatgagtctcaggaaattgatgaagatgaaggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaagaagcagctcaagggcaggagtaatgaaactttgaacaaggtttcagttcttagtttgtaaggtatattacattttatattcatttatgatagcagacaaccttttaagattgctttaattagtatctgatgttgatttttaagtggcattcttttccttaggactttttatgtatacctgttgattgttgtgtaaattttagtaaatctaagagagtgtactaaaccagcaggtatctgttagcttatgtgtttaattgaaattagaaggctaagatggtataacagcattttattgctttgtccagctacaacttgtcatttttttctccatgtcttatcttcctgtttcactttagtttattcttcgttttttattgagatctataaaaaattggcttacttaatagcaaattacttgaagaatttgcctgctttatataaagttagcactttaagatttttttttttagagatgagaagacatttaaattgaagaaaaattcccccagcaatagacagtctatcagtccaagtatttacttcctgagttttgatcaatattttttatttgtgtatgttaatcgtcataaaaacagtgattttggtgtgttttttattttggtgctttaatggcttaagatgttgcacattttttttttcttttggtttctgtttatgtttttttgcctatgcagttaaatttttcctagaaatagcatttgtgttgaacagtaacactttatacatatatatatgcatgtttattttgtttggcgtctttggagggatgcttttagacttgtttgcaaaagggcagttttctttttctttgctgcagttgtctattttgcagaataatagtgtgtgcaagtttgtgagcaaatgaaatatgcaggttcaatctattgattttgatttttacatcttatatctatgccagaatctgtatttcatataacttatttatttcgaatggatgtagtaaattcacagctatcagttttgattttgcaataaataaaccactaggttgcatgtcgaacaaatttttatctcaaataccaaccatcagttttttttttcatgtgttttggtacagctaattcctaattgtagagtgttaaatgtttgaggagaaccttttctcatagatggttggtgttcatatggctactttacaataaagagaactgtaagtgatatttggaaactacaaacctggaattaggagatataattattccttcaagttttatagaatatcacttgggagattccaaagccatagctattacgcggcaaacctaggataagaaaggtagtatgagtgctggtagaccagctgcaactttcctatacagtgaaaaaggctggtgaaacaagtacagtccagattttttaaaatcatactttctcagggatctccacaaactggtgggtgtcctggctgtctgtgtgatagcctctttctataggtgaggcctcaaatgaattgcagctatcctggtgttcctatgagggcactttgtatgaaaaagggcatgtactccaaaacatttttgtaggttctttggccagttgccaaagagtgtgaaagaatccaatagaggatttttcttactgatagcagtcattcattgcagtaaaataaaatatgatcccattagggaatcttgaattctgacctcccatactccgttttgaaataaccactttatatttcattttttaaaaatctgatgatctctttgaggcaggtttcagatttggcagtacaacatgaaagattaggaaaagcattaataacgtgtgggtggaaagcttgttaaaaatctgagagtgaagtttgagttaaaagttgtttgaacatggcattgactgggaggccaaagatttaaagaagcggaagattcttctcttaagacatgaggagtaagttgtgtgataatggtatgtgttttgtgtgcatgaatggacattgtaaatgttgaattctaggctccgacaatcattgtcaacagaagatcaagctgcaaatatttatgttttaaaacttaaattataaagctagttaagtctttctaatgactagttttaatgttcatgggtacattttacctaagttaccgtttacattgtatagaaaaagatacatcttaagcacagattggttattaggaattagtttggggaagaggtttttttgtggattctttcatactgcaaagaaaaaccatttgccttttggggaattgagctaacttctaatctagtcttaagactagaatgctaaaaacaaaaacatgaaggaaattaaaaccccttattattaaattgatttgtaaaaacattgttactggaaatttattggacttgaggccttcctccagaaaataaggacttgattgtcaggcctatattaggttctgaaccttaatgccatgtatttgtacttactaaaaattgtttcaatgaaaagtacattagcagtatgaacttctggtccagttggaagtttttccatttgaaaaatgtgatgtttgcatggaactgtttgaaacttttttattttctagtccccctcccccacactggatagaatttagcctagaattttccctttggataaaagaacaaaaattgaacatgttatttgtaaattgatgtttagtaattagtgataaacttgaaatactagcatatattataagccttaatcttaggtagtcttatgaaaatgaatctcttaactatcttttgaacctgtattcacattggttttcaagatattttaagttatattttttcctcttttcagagctgcttcttattctggggctacttttttttttagttgtgtaattcacaaagggctgcatttttttttttttttaataaggcttataactatggctggatcttttgctctagtcttctaagaagggccattttattttttagagtcacttctaaagtcatgtggtaattaactttggagactgttttgcgtatgagtgctgatacaaattaaaacccaagtagacctcattgcatgtcaccctatgaatgttgacaatggaaggaataccttgcctgtagtatactgtcacttctggattgataagctgaggaagaaagttaagtttcttttttacataagtcagaaaaacttacagctggtgttcctagtttcctggttgacctcagcagatgaagtgaacagatagtgttaattcagattgaagaaattatctgaatcttggtttgtgtagatttacaatctacatgcaatattaactaaatcagatagcttttacagtttcacatgtgtacataggttccctcccggtcccttccatatccattagttattgaactttctaaactggcattgaaacattacaacaatgttttgttgcaccaattttataaacttaagcagtgcaatacgtgttacttttctgaggcaaaccaaaggtaaatttctcaaggttcttgctgccttctttagcagcatttgatggaagatcttttatacatttgtaatagataaaaataaaccagattgcaaatccttttttaaaatcctaaaccatgtaccaagtttttggtccaaattatgtaggataagttaaacttaaattgcattctattaaccaatatgagtgtatttctgtaagcatagttatgttgaaataaagttttaaaaacca

SEQ ID NO: 59 - DNA, 325 bp, Artificial Sequence
  Description of Artificial Sequence Synthetic polynucleotide
gcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacag

SEQ ID NO: 60 - DNA, 84 bp, Artificial Sequence; identical to SEQ ID NO: 57
  Description of Artificial Sequence Synthetic oligonucleotide

SEQ ID NO: 61 - DNA, 50 bp, Artificial Sequence
  Description of Artificial Sequence Synthetic oligonucleotide
tggggtatggataccatttggtaatatttactagagtgtgatctagatgg

SEQ ID NO: 62 - DNA, 23 bp, Artificial Sequence
  Description of Artificial Sequence Synthetic oligonucleotide
gtatggataccatttggtaatat

SEQ ID NO: 63 - DNA, 19 bp, Artificial Sequence
  Description of Artificial Sequence Synthetic oligonucleotide
gtgtgatctagatgggtga

SEQ ID NO: 64 - DNA, 19 bp, Artificial Sequence
  Description of Artificial Sequence Synthetic oligonucleotide
tgtgatctagatgggtgag

SEQ ID NO: 65 - DNA, 119 bp, Artificial Sequence
  Description of Artificial Sequence Synthetic polynucleotide
gtgagaagccatttaaatgtgatcagtgcagttatgtggcctctaatcaacatgaagtaacccgccatgcaagacaggttcacaatgggcctaaacctcttaattgcccacactgtgat

SEQ ID NO: 66 - DNA, 398 bp, Artificial Sequence
  Description of Artificial Sequence Synthetic polynucleotide
gagtgtcggggcgactcccgcgagttggtgtgtaaatgtgtgcagtgagcgggccgaggcgagaggaagcgagagcgcccgagtttgcaaagagctgctttgtgtttggtattttgagaaaatggccgaatgcctatttcagttaggaatgccgccgccgaggcaggcgctggtggcagcagcacggaggaattcgtgcagcacgttgggctttcgggctgtgcctggacggtcttcctaacaatcgttaaaattccatttggggtggcagttaggggttagggagcaattagaaaagtcgatgttgggccaaattacccaatagcttgggctgttcagccccgggacagccttcgccctctgttttctgtgatggcatttgcttccaacttttgttg

SEQ ID NO: 67 - DNA, 59 bp, Artificial Sequence
  Description of Artificial Sequence Synthetic oligonucleotide
aatacagttatggccacccaggtaatggggcagtcttctggaggaggagggctgtttac

SEQ ID NO: 68 - DNA, 127 bp, Artificial Sequence
  Description of Artificial Sequence Synthetic polynucleotide
cggcgctcggagcccgacgcctcgcgagggcgcccgcggagcctccccggccctgggcgttgggcgagccccggggcggtcggaggggcccgggggcggtcggagaagcccggacgccggctgcgcg

SEQ ID NO: 69 - DNA, 386 bp, Artificial Sequence
  Description of Artificial Sequence Synthetic polynucleotide
aatacagttatggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttga